SEQUENCE LISTING (194 sequences)

SEQ ID NO: 1 - protein, 4 aa, synthetic construct
  REGION 1..4: note = linker
GGGS

SEQ ID NO: 2 - protein, 6 aa, synthetic construct
  REGION 1..6: note = linker
GGSGGS

SEQ ID NO: 3 - protein, 9 aa, synthetic construct
  REGION 1..9: note = linker
GGSGGSGGS

SEQ ID NO: 4 - protein, 18 aa, synthetic construct
  REGION 1..18: note = linker
GGSGGSGGSGGSGGSGGS

SEQ ID NO: 5 - protein, 21 aa, synthetic construct
  REGION 1..21: note = linker
GGSGGSGGSGGSGGSGGSGGS

SEQ ID NO: 6 - protein, 5 aa, synthetic construct
  REGION 1..5: note = linker
GGGGS

SEQ ID NO: 7 - protein, 10 aa, synthetic construct
  REGION 1..10: note = linker
GGGGSGGGGS

SEQ ID NO: 8 - protein, 20 aa, synthetic construct
  REGION 1..20: note = linker
GGGGSGGGGSGGGGSGGGGS

SEQ ID NO: 9 - protein, 30 aa, synthetic construct
  REGION 1..30: note = linker
GGGGSGGGGSGGGGSGGGGSGGGGSGGGGS

SEQ ID NO: 10 - protein, 15 aa, Homo sapiens
EPKSCDKTHTCPPCP

SEQ ID NO: 11 - protein, 10 aa, Homo sapiens
EPKSCDKTHT

SEQ ID NO: 12 - protein, 10 aa, Homo sapiens
DKTHTCPPCP

SEQ ID NO: 13 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 14 - protein, 254 aa, Macaca fascicularis
MWQLLLPTALLLLVSAGMRAEDLPKAVVFLEPQWYRVLEKDRVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQVSFCLVMVLLFAVDTGLYFSMKKSIPSSTRDWEDHKFKWSKDPQDK

SEQ ID NO: 15 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 16 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 17 - protein, 201 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 18 - protein, 276 aa, Homo sapiens
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 19 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 20 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 21 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 22 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 23 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 24 - protein, 287 aa, Homo sapiens
MDPKQTTLLCLVLCLGQRIQAQEGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGENISLTCSSAHIPFDRFSLAKEGELSLPQHQSGEHPANFSLGPVDLNVSGIYRCYGWYNRSPYLWSFPSNALELVVTDSIHQDYTTQNLIRMAVAGLVLVALLAILVENWHSHTALNKEASADVAEPSWSQQMCQPGLTFARTPSVCK

SEQ ID NO: 25 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 26 - protein, 15 aa, Homo sapiens
EPKSCDKTHTCPPCP

SEQ ID NO: 27 - protein, 10 aa, Homo sapiens
EPKSCDKTHT

SEQ ID NO: 28 - protein, 10 aa, Homo sapiens
DKTHTCPPCP

SEQ ID NO: 29 - protein, 12 aa, Homo sapiens
ERKCCVECPPCP

SEQ ID NO: 30 - protein, 16 aa, Homo sapiens
ELKTPLDTTHTCPRCP

SEQ ID NO: 31 - protein, 17 aa, Homo sapiens
ELKTPLGDTTHTCPRCP

SEQ ID NO: 32 - protein, 12 aa, Homo sapiens
ESKYGPPCPSCP

SEQ ID NO: 33 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 107 aa, Homo sapiens [sequence omitted]

SEQ ID NO: 38 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 39 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 216 aa, synthetic construct
  REGION 1..216: note = antibody fragment
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 216 aa, synthetic construct
  REGION 1..216: note = antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 44 - protein, 216 aa, synthetic construct
  REGION 1..216: note = antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 216 aa, synthetic construct
  REGION 1..216: note = antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 46 - protein, 216 aa, synthetic construct
  REGION 1..216: note = antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 48 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 50 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 52 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 54 - protein, 216 aa, synthetic construct
  REGION 1..216: note = antibody fragment
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 216 aa, synthetic construct
  REGION 1..216: note = antibody fragment
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 56 - protein, 216 aa, synthetic construct
  REGION 1..216: note = antibody fragment
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 216 aa, synthetic construct
  REGION 1..216: note = antibody fragment
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 58 - protein, 216 aa, synthetic construct
  REGION 1..216: note = antibody fragment
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 120 aa, synthetic construct
  REGION 1..120: note = antibody VH region
QVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 60 - protein, 120 aa, synthetic construct
  REGION 1..120: note = antibody VH region
QVQLVQSGAEVKKPGESLKVSCKASGYTFTNYYMQWVRQAPGQGLEWMGIINPSGGVTSYAQKFQGRVTMTRDTSTSTVYMELSSLRSEDTAVYYCARGSAYYYDFADYWGQGTLVTVSS

SEQ ID NO: 61 - protein, 120 aa, synthetic construct
  REGION 1..120: note = antibody VH region
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 62 - protein, 120 aa, synthetic construct
  REGION 1..120: note = antibody VH region
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 63 - protein, 121 aa, synthetic construct
  REGION 1..121: note = antibody VH region
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 64 - protein, 121 aa, synthetic construct
  REGION 1..121: note = antibody VH region
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSS

SEQ ID NO: 65 - protein, 121 aa, synthetic construct
  REGION 1..121: note = antibody VH region
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 66 - protein, 120 aa, synthetic construct
  REGION 1..120: note = antibody VH region
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSS

SEQ ID NO: 67 - protein, 120 aa, synthetic construct [sequence omitted]

SEQ ID NO: 68 - protein, 106 aa, synthetic construct
  REGION 1..106: note = antibody VL region
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 69 - protein, 106 aa, synthetic construct
  REGION 1..106: note = antibody VL region
SYVLTQPSSVSVAPGQTARITCGGNNIGSKSVHWYQQKPGQAPVLVIYQDKKRPSGIPERFSGSNSGNTATLTISGTQAMDEADYYCQVWDDYIVLFGGGTKLTVL

SEQ ID NO: 70 - protein, 106 aa, synthetic construct; identical to SEQ ID NO: 68
  REGION 1..106: note = antibody VL region

SEQ ID NO: 71 - protein, 107 aa, synthetic construct
  REGION 1..107: note = antibody VL region
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 72 - protein, 110 aa, synthetic construct
  REGION 1..110: note = antibody VL region
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 73 - protein, 107 aa, synthetic construct
  REGION 1..107: note = antibody VL region
AIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIK

SEQ ID NO: 74 - protein, 112 aa, synthetic construct
  REGION 1..112: note = antibody VL region
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 75 - protein, 108 aa, synthetic construct
  REGION 1..108: note = antibody VL region
QPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVL

SEQ ID NO: 76 - protein, 107 aa, synthetic construct [sequence omitted]

SEQ ID NO: 77 - protein, 5 aa, synthetic construct
  REGION 1..5: note = antibody CDR
SYYMH

SEQ ID NO: 78 - protein, 17 aa, synthetic construct
  REGION 1..17: note = antibody CDR
AIEPMYGSTSYAQKFQG

SEQ ID NO: 79 - protein, 11 aa, synthetic construct
  REGION 1..11: note = antibody CDR
GSAYYYDFADY

SEQ ID NO: 80 - protein, 11 aa, synthetic construct
  REGION 1..11: note = antibody CDR
GGHNIGSKNVH

SEQ ID NO: 81 - protein, 7 aa, synthetic construct
  REGION 1..7: note = antibody CDR
QDNKRPS

SEQ ID NO: 82 - protein, 9 aa, synthetic construct
  REGION 1..9: note = antibody CDR
QVWDNYSVL

SEQ ID NO: 83 - protein, 5 aa, synthetic construct
  REGION 1..5: note = antibody CDR
NYYMQ

SEQ ID NO: 84 - protein, 17 aa, synthetic construct
  REGION 1..17: note = antibody CDR
IINPSGGVTSYAQKFQG

SEQ ID NO: 85 - protein, 11 aa, synthetic construct
  REGION 1..11: note = antibody CDR
GSAYYYDFADY

SEQ ID NO: 86 - protein, 11 aa, synthetic construct
  REGION 1..11: note = antibody CDR
GGNNIGSKSVH

SEQ ID NO: 87 - protein, 7 aa, synthetic construct
  REGION 1..7: note = antibody CDR
QDKKRPS

SEQ ID NO: 88 - protein, 9 aa, synthetic construct
  REGION 1..9: note = antibody CDR
QVWDDYIVL

SEQ ID NO: 89 - protein, 17 aa, synthetic construct
  REGION 1..17: note = antibody CDR
IINPSGGSTSYAQKFQG

SEQ ID NO: 90 - protein, 5 aa, synthetic construct
  REGION 1..5: note = antibody CDR
DYVIN

SEQ ID NO: 91 - protein, 17 aa, synthetic construct
  REGION 1..17: note = antibody CDR
EIYPGSGTNYYNEKFKA

SEQ ID NO: 92 - protein, 11 aa, synthetic construct
  REGION 1..11: note = antibody CDR
RGRYGLYAMDY

SEQ ID NO: 93 - protein, 11 aa, synthetic construct
  REGION 1..11: note = antibody CDR
RASQDISNYLN

SEQ ID NO: 94 - protein, 7 aa, synthetic construct
  REGION 1..7: note = antibody CDR
YTSRLHS

SEQ ID NO: 95 - protein, 9 aa, synthetic construct
  REGION 1..9: note = antibody CDR
QQGNTRPWT

SEQ ID NO: 96 - protein, 5 aa, synthetic construct
  REGION 1..5: note = antibody CDR
SYGMH

SEQ ID NO: 97 - protein, 17 aa, synthetic construct
  REGION 1..17: note = antibody CDR
FIRYDGSNKYYADSVKG

SEQ ID NO: 98 - protein, 12 aa, synthetic construct
  REGION 1..12: note = antibody CDR
DRGLGDGTYFDY

SEQ ID NO: 99 - protein, 13 aa, synthetic construct
  REGION 1..13: note = antibody CDR
SGSSSNIGNNAVN

SEQ ID NO: 100 - protein, 7 aa, synthetic construct
  REGION 1..7: note = antibody CDR
YDDLLPS

SEQ ID NO: 101 - protein, 11 aa, synthetic construct
  REGION 1..11: note = antibody CDR
AAWDDSLNGPV

SEQ ID NO: 102 - protein, 5 aa, synthetic construct
  REGION 1..5: note = antibody CDR
NYDMA

SEQ ID NO: 103 - protein, 17 aa, synthetic construct
  REGION 1..17: note = antibody CDR
SISTRGDITSYRDSVKG

SEQ ID NO: 104 - protein, 12 aa, synthetic construct
  REGION 1..12: note = antibody CDR
QDYYTDYMGFAY

SEQ ID NO: 105 - protein, 11 aa, synthetic construct
  REGION 1..11: note = antibody CDR
RASEDIYNGLA

SEQ ID NO: 106 - protein, 7 aa, synthetic construct
  REGION 1..7: note = antibody CDR
GASSLQD

SEQ ID NO: 107 - protein, 9 aa, synthetic construct
  REGION 1..9: note = antibody CDR
AGPHKYPLT

SEQ ID NO: 108 - protein, 5 aa, synthetic construct
  REGION 1..5: note = antibody CDR
SYVMH

SEQ ID NO: 109 - protein, 17 aa, synthetic construct
  REGION 1..17: note = antibody CDR
YINPYNDGTKYNEKFQG

SEQ ID NO: 110 - protein, 12 aa, synthetic construct
  REGION 1..12: note = antibody CDR
GTYYYGTRVFDY

SEQ ID NO: 111 - protein, 16 aa, synthetic construct
  REGION 1..16: note = antibody CDR
RSSKSLQNVNGNTYLY

SEQ ID NO: 112 - protein, 7 aa, synthetic construct
  REGION 1..7: note = antibody CDR
RMSNLNS

SEQ ID NO: 113 - protein, 9 aa, synthetic construct
  REGION 1..9: note = antibody CDR
MQHLEYPIT

SEQ ID NO: 114 - protein, 7 aa, synthetic construct
  REGION 1..7: note = antibody CDR
SGSYYWS

SEQ ID NO: 115 - protein, 16 aa, synthetic construct
  REGION 1..16: note = antibody CDR
YIYYSGSTNYNPSLKS

SEQ ID NO: 116 - protein, 10 aa, synthetic construct
  REGION 1..10: note = antibody CDR
NPISIPAFDI

SEQ ID NO: 117 - protein, 11 aa, synthetic construct
  REGION 1..11: note = antibody CDR
GGNNIGSKSVH

SEQ ID NO: 118 - protein, 7 aa, synthetic construct
  REGION 1..7: note = antibody CDR
YDSDRPS

SEQ ID NO: 119 - protein, 11 aa, synthetic construct
  REGION 1..11: note = antibody CDR
QVWDTSSDHVL

SEQ ID NO: 120 - protein, 5 aa, synthetic construct
  REGION 1..5: note = antibody CDR
DTYIH

SEQ ID NO: 121 - protein, 17 aa, synthetic construct
  REGION 1..17: note = antibody CDR
RIYPTNGYTRYADSVKG

SEQ ID NO: 122 - protein, 11 aa, synthetic construct
  REGION 1..11: note = antibody CDR
WGGDGFYAMDY

SEQ ID NO: 123 - protein, 11 aa, synthetic construct
  REGION 1..11: note = antibody CDR
RASQDVNTAVA

SEQ ID NO: 124 - protein, 7 aa, synthetic construct
  REGION 1..7: note = antibody CDR
SASFLYS

SEQ ID NO: 125 - protein, 9 aa, synthetic construct
  REGION 1..9: note = antibody CDR
QQHYTTPPT

SEQ ID NO: 126 - protein, 482 aa, synthetic construct
  REGION 1..482: note = antibody construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 127 - protein, 482 aa, synthetic construct
  REGION 1..482: note = antibody construct
SYVLTQPSSVSVAPGQTARITCGGNNIGSKSVHWYQQKPGQAPVLVIYQDKKRPSGIPERFSGSNSGNTATLTISGTQAMDEADYYCQVWDDYIVLFGGGTKLTVLGGSGGSQVQLVQSGAEVKKPGESLKVSCKASGYTFTNYYMQWVRQAPGQGLEWMGIINPSGGVTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTARITCGGNNIGSKSVHWYQQKPGQAPVLVIYQDKKRPSGIPERFSGSNSGNTATLTISGTQAMDEADYYCQVWDDYIVLFGGGTKLTVLGGSGGSQVQLVQSGAEVKKPGESLKVSCKASGYTFTNYYMQWVRQAPGQGLEWMGIINPSGGVTSYAQKFQGRVTMTRDTSTSTVYMELSSLRSEDTAVYYCARGSAYYYDFADYWGQGTLVTVSS

SEQ ID NO: 128 - protein, 487 aa, synthetic construct
  REGION 1..487: note = antibody construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSGGSGGSGGSGGSGGSGGSGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 129 - protein, 492 aa, synthetic construct
  REGION 1..492: note = antibody construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGSGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGSGGSGGSGGSGGSGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGSGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 130 - protein, 247 aa, synthetic construct
  REGION 1..247: note = antibody fragment
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSGGSGGSGGSGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 131 - protein, 247 aa, synthetic construct
  REGION 1..247: note = antibody fragment
SYVLTQPSSVSVAPGQTARITCGGNNIGSKSVHWYQQKPGQAPVLVIYQDKKRPSGIPERFSGSNSGNTATLTISGTQAMDEADYYCQVWDDYIVLFGGGTKLTVLGGSGGSGGSGGSGGSGGSGGSQVQLVQSGAEVKKPGESLKVSCKASGYTFTNYYMQWVRQAPGQGLEWMGIINPSGGVTSYAQKFQGRVTMTRDTSTSTVYMELSSLRSEDTAVYYCARGSAYYYDFADYWGQGTLVTVSS

SEQ ID NO: 132 - protein, 248 aa, synthetic construct
  REGION 1..248: note = antibody fragment
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSGGSGGSGGSGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 133 - protein, 252 aa, synthetic construct
  REGION 1..252: note = antibody fragment
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGSGGSGGSGGSGGSGGSGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 134 - protein, 228 aa, synthetic construct
  REGION 1..228: note = antibody fragment
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 135 - protein, 212 aa, synthetic construct
  REGION 1..212: note = antibody fragment
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 136 - protein, 229 aa, synthetic construct
  REGION 1..229: note = antibody fragment
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 137 - protein, 214 aa, synthetic construct
  REGION 1..214: note = antibody fragment
AIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 229 aa, synthetic construct
  REGION 1..229: note = antibody fragment
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 139 - protein, 219 aa, synthetic construct
  REGION 1..219: note = antibody fragment
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 228 aa, synthetic construct
  REGION 1..228: note = antibody fragment
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 141 - protein, 214 aa, synthetic construct
  REGION 1..214: note = antibody fragment
QPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 142 - protein, 228 aa, synthetic construct
  REGION 1..228: note = antibody fragment
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 143 - protein, 214 aa, synthetic construct
  REGION 1..214: note = antibody fragment
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 246 aa, synthetic construct
  REGION 1..246: note = antibody fragment
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGSGGSGGSGGSGGSGGSAIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIK

SEQ ID NO: 145 - protein, 251 aa, synthetic construct
  REGION 1..251: note = antibody fragment
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSGGSGGSGGSGGSGGSGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 146 - protein, 246 aa, synthetic construct
  REGION 1..246: note = antibody fragment
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGSGGSGGSGGSGGSGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVL

SEQ ID NO: 147 - protein, 245 aa, synthetic construct
  REGION 1..245: note = antibody fragment
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSGGSGGSGGSGGSGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 148 - protein, 966 aa, synthetic construct
  REGION 1..966: note = antibody construct
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIKEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 149 - protein, 969 aa, synthetic construct
  REGION 1..969: note = antibody construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGSGGSGGSGGSGGSGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 150 - protein, 724 aa, synthetic construct
  REGION 1..724: note = antibody construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGGGSDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGAAAGSHHHHHH

SEQ ID NO: 151 - protein, 966 aa, synthetic construct
  REGION 1..966: note = antibody construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGGGSDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIK

SEQ ID NO: 152 - protein, 729 aa, synthetic construct
  REGION 1..729: note = antibody construct
DKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSAAAGSHHHHHH

SEQ ID NO: 153 - protein, 972 aa, synthetic construct
  REGION 1..972: note = antibody construct
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIKDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSAAAGSHHHHHH

SEQ ID NO: 154 - protein, 729 aa, synthetic construct; identical to SEQ ID NO: 152
  REGION 1..729: note = antibody construct

SEQ ID NO: 155 - protein, 966 aa, synthetic construct; identical to SEQ ID NO: 151
  REGION 1..966: note = antibody construct

SEQ ID NO: 156 - protein, 724 aa, synthetic construct; identical to SEQ ID NO: 150
  REGION 1..724: note = antibody construct

SEQ ID NO: 157 - protein, 976 aa, synthetic construct
  REGION 1..976: note = antibody construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGGGSDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGSGGSGGSGGSGGSGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLGAAEPEA

SEQ ID NO: 158 - protein, 729 aa, synthetic construct; identical to SEQ ID NO: 152
  REGION 1..729: note = antibody construct

SEQ ID NO: 159 - protein, 971 aa, synthetic construct
  REGION 1..971: note = antibody construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGSGGSGGSGGSGGSGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGAAEPEA

SEQ ID NO: 160 - protein, 729 aa, synthetic construct; identical to SEQ ID NO: 152
  REGION 1..729: note = antibody construct

SEQ ID NO: 161 - protein, 976 aa, synthetic construct; identical to SEQ ID NO: 157
  REGION 1..976: note = antibody construct

SEQ ID NO: 162 - protein, 941 aa, synthetic construct
  REGION 1..941: note = antibody construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSGGSGGSGGSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 163 - protein, 214 aa, synthetic construct; identical to SEQ ID NO: 141
  REGION 1..214: note = antibody construct

SEQ ID NO: 164 - protein, 449 aa, synthetic construct
  REGION 1..449: note = antibody heavy chain
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 214 aa, synthetic construct; identical to SEQ ID NO: 141
  REGION 1..214: note = antibody light chain

SEQ ID NO: 166 - protein, 452 aa, synthetic construct
  REGION 1..452: note = antibody heavy chain
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 214 aa, synthetic construct
  REGION 1..214: note = antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 449 aa, synthetic construct
  REGION 1..449: note = antibody heavy chain
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 214 aa, synthetic construct; identical to SEQ ID NO: 141
  REGION 1..214: note = antibody light chain

SEQ ID NO: 170 - protein, 705 aa, synthetic construct
  REGION 1..705: note = antibody construct
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGSGGSGGSGGSGGSGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVL

SEQ ID NO: 171 - protein, 212 aa, synthetic construct; identical to SEQ ID NO: 135
  REGION 1..212: note = antibody construct

SEQ ID NO: 172 - protein, 727 aa, synthetic construct
  REGION 1..727: note = antibody construct
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSGGSGGSGGSGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 173 - protein, 214 aa, synthetic construct; identical to SEQ ID NO: 137
  REGION 1..214: note = antibody construct

SEQ ID NO: 174 - protein, 726 aa, synthetic construct
  REGION 1..726: note = antibody construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSGGSGGSGGSGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 175 - protein, 214 aa, synthetic construct; identical to SEQ ID NO: 141
  REGION 1..214: note = antibody construct

SEQ ID NO: 176 - protein, 727 aa, synthetic construct
  REGION 1..727: note = antibody construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSGGSGGSGGSGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 177 - protein, 219 aa, synthetic construct; identical to SEQ ID NO: 139
  REGION 1..219: note = antibody construct

SEQ ID NO: 178 - protein, 726 aa, synthetic construct
  REGION 1..726: note = antibody construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYVLTQPSSVSVAPGQTARITCGGNNIGSKSVHWYQQKPGQAPVLVIYQDKKRPSGIPERFSGSNSGNTATLTISGTQAMDEADYYCQVWDDYIVLFGGGTKLTVLGGSGGSGGSGGSGGSGGSGGSQVQLVQSGAEVKKPGESLKVSCKASGYTFTNYYMQWVRQAPGQGLEWMGIINPSGGVTSYAQKFQGRVTMTRDTSTSTVYMELSSLRSEDTAVYYCARGSAYYYDFADYWGQGTLVTVSS

SEQ ID NO: 179 - protein, 214 aa, synthetic construct; identical to SEQ ID NO: 141
  REGION 1..214: note = antibody construct

SEQ ID NO: 180 - protein, 946 aa, synthetic construct
  REGION 1..946: note = antibody construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSGGSGGSGGSGGSGGSGGSGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 181 - protein, 214 aa, synthetic construct; identical to SEQ ID NO: 143
  REGION 1..214: note = antibody construct

SEQ ID NO: 182 - protein, 946 aa, synthetic construct
  REGION 1..946: note = antibody construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSGGSGGSGGSGGSGGSGGSGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 183 - protein, 214 aa, synthetic construct; identical to SEQ ID NO: 143
  REGION 1..214: note = antibody construct

SEQ ID NO: 184 - protein, 727 aa, synthetic construct
  REGION 1..727: note = antibody construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSGGGGSGGGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSGGSGGSGGSGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 185 - protein, 214 aa, synthetic construct; identical to SEQ ID NO: 143
  REGION 1..214: note = antibody construct

SEQ ID NO: 186 - protein, 727 aa, synthetic construct
  REGION 1..727: note = antibody construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGSGGSGGSGGSGGSGGSGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 187 - protein, 214 aa, synthetic construct; identical to SEQ ID NO: 143
  REGION 1..214: note = antibody construct

SEQ ID NO: 188 - protein, 705 aa, synthetic construct
  REGION 1..705: note = antibody construct
QVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGSGGSGGSGGSGGSGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVL

SEQ ID NO: 189 - protein, 212 aa, synthetic construct; identical to SEQ ID NO: 135
  REGION 1..212: note = antibody construct

SEQ ID NO: 190 - protein, 969 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGSGGSGGSGGSGGSGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLGGGGSDKTHTCPPCPAPEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVSSGGSGGSGGSGGSGGSGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVSS

SEQ ID NO: 191 - protein, 941 aa, synthetic construct
EVQLVESGGGLVQPGGSLRLSCAASGVSLPDYGVSWVRQAPGKGLEWIGVIWGSETTYYNSALKSKFIISRDNAKNSLYLQMNSLRAEDTAVYYCARHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVSSGGSGGSGGSGGSGGSGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVSS

SEQ ID NO: 192 - protein, 214 aa, synthetic construct
DIQMTQSPSSLSASVGDRVTITCRASQDISKYLNWYQQKPGKVPKLLIYHTSRLHSGVPDRFSGSGSGTDFTLTISSLQPEDVATYYCQQGNTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 726 aa, synthetic construct
EVQLVESGGGLVQPGGSLRLSCAASGVSLPDYGVSWVRQAPGKGLEWIGVIWGSETTYYNSALKSKFIISRDNAKNSLYLQMNSLRAEDTAVYYCARHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVSS

SEQ ID NO: 194 - protein, 214 aa, synthetic construct; identical to SEQ ID NO: 192

(19) United States
(12) Patent Application Publication
(10) Pub. No.: US 2024/0376208 A1
(43) Pub. Date: Nov. 14, 2024

(54) DUPLEXBODIES

(71) Applicant: Affimed GmbH, Mannheim (DE)

(72) Inventors: Uwe REUSCH, Mannheim (DE); Joachim KOCH, Mannheim (DE)

(21) Appl. No.: 18/293,307

(22) PCT Filed: Aug. 1, 2022
(86) PCT No.: PCT/EP2022/071490

§ 371 Date: Jan. 29, 2024

(30) Foreign Application Priority Data
Jul. 30, 2021 (EP) 21188905.0

(51) Int. Cl. C07K 16/28; A61P 35/00

(52) U.S. Cl. CPC C07K 16/283; A61P 35/00; C07K 16/2803; C07K 16/2833; C07K 16/2851; C07K 16/2863; C07K 16/2866; C07K 16/2875; C07K 16/2878; C07K 16/2887; C07K 16/2893; C07K 2317/31; C07K 2317/92

(57) ABSTRACT

The present invention relates to an antibody construct comprising (i.) at least four first binding domains (A), wherein said first binding domain (A) is capable of specifically binding to a first target (A′) that is an immune-regulatory antigen on the surface of an innate immune effector cell, wherein the immune effector cell is a natural killer cell or a macrophage; and (ii.) a second binding domain (B), which is capable of specifically binding to a second target (B′) that is an antigen on the surface of a target cell. The present invention also relates to related nucleic acid molecules, vectors, host cells, methods of producing the antibody constructs, pharmaceutical compositions, medical uses, and kits.

### FIELD OF THE INVENTION

The present invention relates to an antibody construct comprising (i.) at least four first binding domains (A), wherein said first binding domain (A) is capable of specifically binding to a first target (A′) that is an immune-regulatory antigen on the surface of an innate immune effector cell, wherein the innate immune effector cell is a natural killer cell or a macrophage; and (ii.) a second binding domain (B), which is capable of specifically binding to a second target (B′) that is an antigen on the surface of a target cell. The present invention also relates to related nucleic acid molecules, vectors, host cells, methods of producing the antibody constructs, pharmaceutical compositions, medical uses, and kits.

### BACKGROUND

High expression of tumor antigens (e.g. EGFR, HER2) has been reported in a variety of tumors, which had led to the development of drugs (e.g. monoclonal antibodies) directed against these tumor targets. However, these tumor antigens are often heterogeneously expressed either only higher expressed in a certain tumor subtype or within the tumor tissue (Hoadley et al 2007; Bedard et al 2013; Passaro et al 2020; Zhang et al. 2020). The diverse expression of markers in tumor cells and cancer stem cells reflects the intratumor heterogeneity. Low expression or loss of tumor antigen (e.g. by downregulation or shedding) on tumor cells and cancer stem cells can lead to therapy resistance (Salih et al 2002; Paczulla et al 2019; Reim et al 2009), as tumors might downregulate targeted tumor antigen during therapy as an escape strategy. Common state of the art antitumoral antibodies are more effective in targeting tumor cells with high expression of tumor antigens. However, only targeting tumor cells with high tumor antigen expression will finally lead to outgrowth of the tumor cells with low tumor antigen expression and lack of clinical response to therapy. Tumor cells low for targeted tumor antigen might account for potential to relapse after initial complete remission. Therefore, targeted therapies for several tumor indications remain challenging due to tumor heterogeneity. It is thus object of the invention to provide means and methods for treating tumors, even when the tumor antigen is heterogeneously expressed.

### SUMMARY

The present invention is based on the surprising finding that a bispecific antibody construct having at least four binding domains specific for an immune regulatory antigen on the surface of an innate immune effector cell and at least one binding domain for an antigen on the surface of a target cell can efficiently kill target cells even with low or very low expression of the target antigen.

As shown in Example 10 of the present specification, bispecific antibodies having one or two binding domains for EGFR and four binding domains for CD16A have a surprisingly increased potency and efficacy against Daudi cells, which have a very low expression of EGFR, as compared to antibodies having only two binding domains for CD16A.

The antibody constructs of the invention can thus be useful for tumor therapy, because they are not only capable of removing cells in a tumor that have high expression of the target antigen, but also those cells that have low or very low expression of the target antigen, including tumor stem cells. Thus, the antibody construct can target the entire tumor. The antibody constructs of the invention can thus be useful for targeting tumors with heterologous expression of the target antigen.

Thus, the present invention relates to a bispecific antibody construct comprising (i.) at least four first binding domains (A), wherein said first binding domain (A) is capable of specifically binding to a first target (A′) that is an immune-regulatory antigen on the surface of an innate immune effector cell, wherein the immune effector cell is a natural killer cell or a macrophage; and (ii.) a second binding domain (B), which is capable of specifically binding to a second target (B′) that is an antigen on the surface of a target cell.

The present invention also relates to a nucleic acid molecule comprising a sequence encoding an antibody construct of the invention.

The present invention also relates to a vector comprising a nucleic acid molecule of the invention.

The present invention also relates to a host cell comprising a nucleic acid molecule of the invention or a vector of the invention.

The present invention also relates to a method of producing an antibody construct of the invention, said method comprising culturing a host cell of the invention under conditions allowing the expression of the antibody construct of the invention and optionally recovering the produced antibody construct from the culture.

The present invention also relates to a pharmaceutical composition comprising an antibody construct of the invention, or produced by the method of the invention.

The present invention also relates to an antibody construct of the invention for use in therapy.

The present invention also relates to a method of treatment or amelioration of a proliferative disease, a tumorous disease, a viral disease or an immunological disorder, comprising the step of administering to a subject in need thereof the antibody construct of the invention, or produced by the method of the invention.

The present invention also relates to a method of simultaneously binding a target cell and an immune effector cell, comprising administering to a subject the antibody construct of the invention, wherein the target cell has a low expression of the second target (B′).

The present invention also relates to a kit comprising an antibody construct of the invention, or produced by the method of the invention, a nucleic acid molecule of the invention, a vector of the invention, and/or a host cell of the invention.

### BRIEF DESCRIPTION OF THE DRAWINGS

FIG. 1: Schematic representation of antibody constructs at least four first binding domains (A-E) and reference antibody constructs (F-H).

FIG. 2: Biochemical characterization of antibody constructs. (A) Bi-scDb-IgAb_06 (SEQ ID NOs: 162 and 163), (B) Bi-scDb-Fc_01 (SEQ ID NO: 148), (C) Bi-scDb-Fc_02 (SEQ ID NO: 149), (D) aBi-scDb-Fc_01 (SEQ ID NOs: 150 and 151), (E) aBi-scDb-Fc_02 (SEQ ID NOs: 152 and 153), (F) aBi-scDb-Fc_03 (SEQ ID NOs: 154 and 155), (G) aBi-scDb-Fc_04 (SEQ ID NOs: 156 and 157), (H) aBi-scDb-Fc_05 (SEQ ID NOs: 158 and 159) and (H) aBi-scDb-Fc_06 (SEQ ID NOs: 160 and 161). Left panels: constructs purified via Protein A chromatography and preparative size exclusion chromatography. Right panel: SDS-PAGE analysis under non-reducing (nR) or reducing conditions (R).

FIG. 3: Concentration-dependent lysis of MCF-7 target cells by NK cells. Calcein-labeled MCF-7 cells were co-cultured with enriched primary human NK cells as effector cells at an ET ratio of 5:1 in the presence of serial dilutions of the indicated antibodies. After 4 h incubation the fluorescence of the calcein released from lysed target cells into the supernatant was quantified and used for calculation of % specific lysis. Mean and SD of duplicate values are plotted

FIG. 4: Concentration-dependent lysis of Daudi target cells by NK cells. Calcein-labeled Daudi cells were co-cultured with enriched primary human NK cells as effector cells at an E:T ratio of 5:1 in the presence of serial dilutions of the indicated antibodies. After 4 h incubation the fluorescence of the calcein released from lysed target cells into the supernatant was quantified and used for calculation of % specific lysis. Mean and SD of duplicate values are plotted.

FIG. 5: Concentration-dependent induction of NK cell fratricide by various antibody constructs. Calcein-labeled enriched primary human NK cells were co-cultured with autologous NK cells as effector cells at an ET ratio of 1:1 in the presence of serial dilutions of the indicated antibodies. After 4 h incubation the fluorescence of the calcein released from lysed target cells into the supernatant was quantified and used for calculation of % specific lysis. Anti-CD38 IgG1 (IgAb_51) was used as a positive control. Mean and SD of duplicate values are plotted.

FIG. 6: Concentration-dependent phagocytosis of DK-MG target cells by macrophages. CMFDA-labeled DK-MG cells were co-cultured with human monocyte-derived macrophages as effector cells at an E:T ratio of 5:1 in the presence of serial dilutions of the indicated antibodies. After 4 h incubation, cells were stained with anti-CD11b and viability dye eF780. Phagocytosis of labeled target cells was quantified by analyzing CMFDA⁺/CD11b⁺ cells in % of viable cells by flow cytometry. ADCP in absence of antibodies was used for normalization. Mean and SD of duplicate values are plotted, and one representative experiment is shown.

FIG. 7: Concentration-dependent phagocytosis of EGFR low expressing MCF-7 target cells by macrophages. CMFDA-labeled MCF-7 cells were co-cultured with human monocyte-derived macrophages as effector cells at an E:T ratio of 5:1 in the presence of serial dilutions of the indicated antibodies. After 4 h incubation, cells were stained with anti-CD11b and viability dye eF780. Phagocytosis of labeled target cells was quantified by analyzing CMFDA⁺/CD11b⁺ cells in % of viable cells by flow cytometry. ADCP in absence of antibodies was used for normalization. Mean and SD of duplicate values are plotted, and one representative experiment is shown.

FIG. 8: Concentration-dependent lysis of A-431 target cells by NK cells. Calcein-labeled A-431 cells were co-cultured with enriched primary human NK cells as effector cells at an E:T ratio of 5:1 in the presence of serial dilutions of the indicated antibodies. After 4 h incubation the fluorescence of the calcein released from lysed target cells into the supernatant was quantified and used for calculation of normalized specific lysis. Mean and SD of duplicate values are plotted.

FIG. 9: Concentration-dependent lysis of BCMA⁺ MM.1S cells by NK cells. Calcein-labeled MM.1S cells were co-cultured with enriched primary human NK cells as effector cells at an ET ratio of 5:1 in the presence of serial dilutions of the indicated antibodies. After 4 h incubation the fluorescence of the calcein released from lysed target cells into the supernatant was quantified and used for calculation of % specific lysis. Mean and SD of duplicate values are plotted.

FIG. 10: Scoring of tumor cell lines regarding HER2 and EGFR expression based on specific antibody binding capacity (SABC).

FIG. 11: Concentration-dependent phagocytosis of EGFR expressing HCT-116 target cells by macrophages. CMFDA-labeled HCT-116 cells were co-cultured with human monocyte-derived macrophages as effector cells at an E:T ratio of 5:1 in the presence of serial dilutions of the indicated antibodies. After 4 h incubation, cells were stained with anti-CD11b and viability dye eF780. Phagocytosis of labeled target cells was quantified by analyzing CMFDA⁺/CD11b⁺ cells in % of viable cells by flow cytometry. ADCP in absence of antibodies was used for normalization. Mean and SD of duplicate values are plotted, and one representative experiment is shown.

FIG. 12: Concentration-dependent lysis of CD19⁺ Daudi target cells by NK cells. Calcein-labeled Daudi cells were co-cultured with enriched primary human NK cells as effector cells at an ET ratio of 5:1 in the presence of serial dilutions of the indicated antibodies. After 4 h incubation the fluorescence of the calcein released from lysed target cells into the supernatant was quantified and used for calculation of % specific lysis. Mean and SD of duplicate values are plotted.

### DEFINITIONS

The term “binding domain” characterizes in connection with the present invention a domain which is capable of specifically binding to/interacting with/recognizing a given target epitope or a given target site on the target molecules (antigens), e.g. an antigen on the surface of an innate immune effector cell, such as CD16A or NKp46, and/or e.g. an antigen on the surface of a target cell, respectively. The structure and/or function of the first binding domain (recognizing e.g. an antigen on the surface of an innate immune effector cell), the structure and/or function of the second binding domain (recognizing e.g. an antigen on the surface of a target cell), and also the structure and/or function of the third binding domain (recognizing an antigen on the surface of a target cell), is/are preferably based on the structure and/or function of an antibody, e.g. of a full-length or whole immunoglobulin molecule and/or is/are drawn from the variable heavy chain (VH) and/or variable light chain (VL) domains of an antibody or fragment thereof.

The term “specifically binding”, as used herein means that the binding domain preferentially binds or recognizes the target even when the binding partner is present in a mixture of other molecules or other structures. The binding may be mediated by covalent or non-covalent interactions or a combination of both. In preferred embodiments, “simultaneous binding to a target cell and a immune effector cell” comprises the physical interaction between the binding domains and their targets on the cells, but preferably also includes the induction of an action mediated by the simultaneous binding of the two cells. Such an action may be an immune effector function of the immune effector cell, such as a cytotoxic effect.

The term “antibody construct” refers to a molecule in which the structure and/or function is/are based on the structure and/or function of an antibody, e.g., of a full-length or whole immunoglobulin molecule and/or is/are drawn from the variable heavy chain (VH) and/or variable light chain (VL) domains of an antibody or fragment thereof. An antibody construct is hence capable of specifically binding to its specific target or antigen. Furthermore, the binding region of an antibody construct defined in the context of the invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to a specific antibody competing with the epitope of the defined antibody. The antibodies on which the constructs defined in the context of the invention are based include for example monoclonal, recombinant, chimeric, deimmunized, humanized and human antibodies.

The binding region of an antibody construct defined in the context of the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding region. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge domain; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFv-library).

An antibody construct as defined in the context of the invention may comprise a fragment of a full-length antibody, such as VH, VHH, VL, (s) dAb, Fv, Fd, Fab, Fab′, F(ab′)₂ or “r IgG” (“half antibody”). Antibody constructs as defined in the context of the invention may also comprise modified fragments of antibodies, also called antibody variants, such as scFv, di-scFv or bi(s)-scFv, scFv-Fc, scFv-zipper, scFab, Fab₂, Fab₃, diabodies, single chain diabodies (scDb), tandem diabodies (TandAb's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

As used herein, the terms “single-chain Fv,” “single-chain antibodies” or “scFv” refer to single polypeptide chain antibody fragments that comprise the variable regions from both the heavy and light chains but lack the constant regions. Generally, a single-chain antibody further comprises a polypeptide linker between the VH and VL domains which enables it to form the desired structure which would allow for antigen binding. A preferred linker for this purpose is a glycine serine linker, which preferably comprises from about 15 to about 30 amino acids. Preferred glycine serine linkers may have one or more repeats of GGS, GGGS (SEQ ID NO: 1), or GGGGS (SEQ ID NO: 6). Such linker preferably comprises 5, 6, 7, 8, 9 and/or 10 repeats of GGS, preferably (GGS)₆ (SEQ ID NO: 4) (which are preferably used for scFvs having the arrangement VH-VL), or preferably (GGS)₇ (SEQ ID NO: 5) (which are preferably used for scFvs having the arrangement VL-VH). To stabilize a “single chain antibody” the H44-L100 mutation (Zhao et al., 2010) can be used to introduce an interdomain disulfide bridge. H44 describes the amino acid No. 44 (Kabat numbering) in the VH which has to be changed into a cysteine. Whereas L100 describes the amino acid No. 100 (Kabat numbering) in the VL which has to be changed into a cysteine. Single chain antibodies are discussed in detail by Plueckthun in The Pharmacology of Monoclonal Antibodies, vol. 1 13, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442, Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic. The term “bi-scFv” or “ta-scFv” (tandem scFv) as used herein refers to two scFv that are fused together. Such a bi-scFv or ta-scFv may comprise a linker between the two scFv moieties. Generally, the arrangement of the VH and VL domains on the polypeptide chain within each of the scFv may be in any order. This means that the “bi-scFv” of “ta-scFv” can be arranged in the order VH(1)-VL(1)-VH(2)-VL(2), VL(1)-VH(1)-VH(2)-VL(2), VH(1)-VL(1)-VL(2)-VH(2), or VL(1)-VH(1)-VL(2)-VH(2), where (1) and (2) stand for the first and second scFv, respectively.

The term “double Fab” as used herein refers to two Fab fragments that are fused together, which are preferably staggered. Here, a first chain of a first Fab is N-terminally fused to a first chain of a second Fab, or a second chain of a first Fab is N-terminally fused to a second chain of a second Fab, or both, the first chain of a first Fab and the second chain of a first Fab are fused to first and second chains of a second Fab, respectively. A linker may be present between the fused chains of the first and second Fab. The first and second chains of the first and second Fab can be individually selected from a light chain-derived chain of a Fab (VL-CL), a heavy chain derived chain of a Fab (VH-CH1), as long as each Fab contains a VH, a VL, a CH1, and a CL. As an illustrative example, the light chain-derived chain of the first Fab can be fused to the light chain derived-chain of the second Fab. As another illustrative example, the heavy chain-derived chain of the first Fab can be fused to the heavy chain derived-chain of the second Fab. As a further illustrative example, the heavy chain-derived chain of the first Fab can be fused to the light chain derived-chain of the second Fab. In some double Fabs, both chains of the two Fabs are fused together. For example, the light chain-derived chain of the first Fab can be fused to the light chain derived-chain of the second Fab while the heavy chain-derived chain of the first Fab can be fused to the heavy chain derived-chain of the second Fab. Alternatively, the light chain-derived chain of the first Fab can be fused to the heavy chain derived-chain of the second Fab while the heavy chain-derived chain of the first Fab can be fused to the light chain derived-chain of the second Fab. A fusion of two Fab chains may optionally comprise a linker. Suitable and preferred linkers comprise the upper hinge sequence (SEQ ID NO: 11) or glycine serine linkers with about up to 20 amino acids, preferably up to 10 amino acids, or most preferably 10 amino acids, e.g. two repeats of GGGGS (SEQ ID NO: 7). Glycine serine linkers comprised in a double Fab may have one or more repeats of GGS, GGGS (SEQ ID NO: 1), or GGGGS (SEQ ID NO: 6), such as one, two, three, or four repeats.

As used herein, a “diabody” or “Db” refers to an antibody construct comprising two binding domains, which may be constructed using heavy and light chains disclosed herein, as well as by using individual CDR regions disclosed herein. Typically, a diabody comprise a heavy chain variable domain (VH) connected to a light chain variable domain (VL) by a linker which is too short to allow pairing between the two domains on the same chain. Preferred linkers for this purpose include glycine serine linkers with about up to 12 amino acids, preferably up to about 10 amino acids. Preferred glycine serine linkers may have one or more repeats of GGS, GGGS (SEQ ID NO: 1), or GGGGS (SEQ ID NO: 6). A preferred linker is (GGS) 2 (SEQ ID NO: 2). Another preferred linker is (GGS) 3 (SEQ ID NO: 3). Accordingly, the VH and VL domains of one fragment are forced to pair with the complementary VH and VL domains of another fragment, thereby forming two antigen-binding sites. A diabody can be formed by two separate polypeptide chains, each comprising a VH and a VL. Alternatively, all four variable domains can be comprised in one single polypeptide chain comprising two VH and two VL domains. In such a case, the diabody can also be termed “single chain diabody” or “scDb”. Typically, a scDb comprises the two chains of a non-single chain diabody that are fused together, preferably via a linker. A preferred linker for this purpose is a glycine serine linker, which preferably comprises from about 15 to about 30 amino acids. Preferred glycine serine linkers may have one or more repeats of GGS, GGGS (SEQ ID NO: 1), or GGGGS (SEQ ID NO: 6). Such linker preferably comprises 5, 6, 7, 8, 9, and/or 10 repeats of GGS, preferably (GGS) 6, (SEQ ID NO 4) or preferably (GGS); (SEQ ID NO: 5). On the polypeptide chain, the variable domains of a scDb can be arranged (from N to C terminus) in a VL-VH-VL-VH or VH-VL-VH-VL order. Similarly, the spatial arrangement of the four domains in the tertiary/quaternary structure can be in a VL-VH-VL-VH or VH-VL-VH-VL order. The term diabody does not exclude the fusion of further binding domains to the diabody. Also, “the single chain diabody” can be stabilized by using the H44-L100 mutation (Zhao et al., 2010) to introduce interdomain disulfide bridges. H44 describes the amino acid No. 44 (Kabat numbering) in the VH which has to be changed into a cysteine. Whereas L100 describes the amino acid No. 100 (Kabat numbering) in the VL which has to be changed into a cysteine

Furthermore, the definition of the term “antibody construct” generally includes multivalent constructs, including bispecific constructs, specifically binding to only two antigenic structures, as well as polyspecific/multispecific constructs, which specifically bind more than two antigenic structures, e.g. three, four or more, through distinct binding domains. Antibody constructs of the present invention are multivalent (e.g. pentavalent or hexavalent) antibody constructs that are at least bispecific (such as bispecific or trispecific). Moreover, the definition of the term “antibody construct” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The term “valent” or “valency” denotes the presence of a determined number of antigen-binding domains in the antigen-binding protein. Depending on the context, “valent” or “valency” may be directed to the number of antigen-binding domains that are directed to a particular target, which does not exclude the presence of further antigen binding domains that are specific to other targets. As an illustrative example, a natural IgG has two antigen-binding domains and is bivalent. As another illustrative example the antibody constructs as defined in the context of the invention are at least tetravalent for the first target and comprises at least one further binding domain that is specific for a second target.

The term “bispecific” as used herein refers to an antibody construct which is “at least bispecific”, i.e., it comprises at least a first binding domain and a second binding domain, wherein the first binding domain binds to one antigen or target (here: an antigen on the surface of an innate immune effector cell), the second binding domain binds to another antigen or target (here: an antigen on the surface of target cell). Accordingly, antibody constructs as defined in the context of the invention comprise specificities for at least two different antigens or targets. For example, the first binding domain does preferably bind to an extracellular epitope of an NK cell receptor of one or more of the species selected from human, Macaca spec. and rodent species.

The term “trispecific” as used herein refers to an antibody construct which is “at least trispecific”, i.e., it comprises at least a first binding domain, a second binding domain, and a third binding domain, wherein the first binding domain binds to a first antigen or target (here: an antigen on the surface of an innate immune effector cell), the second binding domain binds to a second antigen or target (here: an antigen on the surface of target cell), and the third binding domain binds to a third antigen or target (here: an antigen on the surface of target cell which is other than the second target). Accordingly, some antibody constructs as defined in the context of the invention comprise specificities for at least three different antigens or targets. For example, the first binding domain does preferably bind to an extracellular epitope of an NK cell receptor of one or more of the species selected from human, Macaca spec. and rodent species.

“CD16A” or “CD16a” refers to the activating receptor CD16A, also known as FcγRIIIA, expressed on the cell surface of NK cells. CD16A is an activating receptor triggering the cytotoxic activity of NK cells. The amino acid sequence of human CD16A is given in UniProt entry P08637 (version 212 of 12 Aug. 2020) as well as in SEQ ID NO: 13. The affinity of antibodies for CD16A directly correlates with their ability to trigger NK cell activation, thus higher affinity towards CD16A reduces the antibody dose required for activation. The antigen-binding site of the antigen-binding protein binds to CD16A, but preferably not to CD16B. For example, an antigen-binding site comprising heavy (VH) and light (VL) chain variable domains binding to CD16A, but not binding to CD16B, may be provided by an antigen-binding site which specifically binds to an epitope of CD16A which comprises amino acid residues of the C-terminal sequence SFFPPGYQ (positions 201-208 of SEQ ID NO:13) and/or residues G147 and/or Y158 of CD16A which are not present in CD16B.

“CD16B” refers to receptor CD16B, also known as FcγRIIIB, expressed on neutrophils and eosinophils. The receptor is glycosylphosphatidyl inositol (GPI) anchored and is understood to not trigger any kind of cytotoxic activity of CD16B positives immune cells.

The term “target cell” describes a cell or a group of cells, which is/are the target of the mode of action applied by the antibody construct of the invention. This cell/group of cells comprise e.g. pathological cells, which are eliminated or inhibited by engaging these cells with the effector cell via the antibody construct of the invention. A preferred target cell is a cancer cell.

The term “target cell surface antigen” or “antigen on the surface of a target cell”, which are used interchangeably, refers to an antigenic structure expressed by a cell and which is present at the cell surface such that it is accessible for an antibody construct as described herein. It may be a protein, preferably the extracellular portion of a protein, a peptide that is presented on the cell surface in an MHC context (including HLA-A2, HLA-A11, HLA-A24, HLA-B44, HLA-C4) or a carbohydrate structure, preferably a carbohydrate structure of a protein, such as a glycoprotein. It is preferably a tumor associated or tumor restricted antigen. It is envisaged that CD16A, CD56, NKG2A, NKG2D, NKp30, NKp44, NKp46, NKp80, DNAM-1, CD89, CD96, CD160, TIGIT, TIM-3, KIR2DL1-5, KIR3DL1-3, and KIR2DS1-5 are not target cell surface antigens according to the present invention.

The term “immune-regulatory antigen” as used herein relates to an antigen which is preferably a receptor. Said antigen or preferably receptor is capable of receiving and/or transducing signals, and its engagement is considered to influence the quality and intensity of the innate immune cell response. Such antigens include inhibitory receptors, activating receptors, adhesion molecules and co-stimulatory molecules. Such “immune-regulatory antigen” includes but is not limited to CD16A, CD56, NKG2A, NKG2D, NKp30, NKp44, NKp46, NKp80, DNAM-1 (CD226), SLAMF7 (CD319), CD244 (2B4), OX40, CD47/SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5, KIR3DS1, and CD3. The term “immune activating antigen” relates to a positive regulator of the immune response of immune cells (e.g. NK cells, macrophages) that can stimulate their functions. The term “immune activating antigen” also includes activating receptors, adhesion molecules and co-stimulatory molecules. Activating receptors often detect self-molecules that are expressed under conditions of cell stress. Some activating receptors signal e.g. through immunoreceptor tyrosine-based activating motifs (ITAMs), which are usually contained in associated molecules, through immunoreceptor tyrosine-based switch motifs (ITSMs) or through other tyrosine-based signaling motifs. Such “immune activating antigen” comprise, but are not limited to CD16A, CD56, NKG2D, NKp30, NKp44, NKp46, NKp80, DNAM-1 (CD226), SLAMF7 (CD319), CD244 (2B4), OX40, CD137, CD89, CD160, and killer-cell immunoglobulin-like receptors (KIR2DS1-5 and KIR3DS1). The term “immune inhibitory antigen” relates to a negative regulator of the immune response of (innate) immune cells (e.g. NK cells, macrophages). An “immune inhibitory antigen” is preferably a receptor. The inhibitory signal is generally transduced through immunoreceptor tyrosine-based inhibitory motifs (ITIMs) located in the intracellular tail of the receptor. Such “immune inhibitory antigen” comprise, but are not limited to NKG2A, TIGIT, PD-1, PD-L1, CD47, SIRPα, LAG-3, CTLA-4, CD96, TIM-3, CD137, KIR2DL1-5 and KIR3DL1-3.

The antibody construct of the invention is at least bispecific but may encompass further specificities resulting in a multispecific antibody constructs such as a trispecific antibody constructs, or constructs having more than three (e.g., four, five, six . . . ) specificities. It is however envisaged, that in these multispecific constructs it is only the first binding domain, which is specific for an antigen on the surface of an innate immune effector cell. Examples for tri- or multispecific antibody constructs are provided e.g. in WO 2015/158636, WO 2017/064221, WO/2019/198051, and Ellwanger et al. (MAbs. 2019 July; 11 (5): 899-918).

Given that the antibody constructs as defined in the context of the invention are (at least) bispecific, they do not occur naturally and they are markedly different from naturally occurring products. A bispecific antibody construct is hence an artificial hybrid antibody having at least two distinct binding sides with different specificities. Bispecific antibody constructs can be produced by a variety of methods including fusion of hybridomas or linking of Fab′ fragments. See, e.g., Songsivilai & Lachmann, Clin. Exp. Immunol. 79:315-321 (1990).

The binding domains and the variable domains (VH/VL) of the antibody construct of the present invention may or may not comprise peptide linkers (spacer peptides). The term “peptide linker” comprises in accordance with the present invention an amino acid sequence by which the amino acid sequences of one (variable and/or binding) domain and another (variable and/or binding) domain of the antibody construct defined herein are linked with each other. The peptide linkers can also be used to fuse one domain to another domain of the antibody construct defined herein. In such cases, the is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or even less. The length of the linker is preferably determined as described by Rossmalen et al Biochemistry 2017, 56, 6565-6574, which also describes suitable linkers that are well known to the skilled person. An example for such a linker is a glycine serine linker or a serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 6), such as (GGGGS)₂ (SEQ ID NO: 7), (GGGGS)₄ (SEQ ID NO: 8), or preferably (GGGGS) (SEQ ID NO: 9). Other illustrative examples for linkers are shown in SEQ ID NOs: 2-5. A preferred technical feature of such peptide linker is that it does not comprise any polymerization activity.

The antibody constructs as defined in the context of the invention are preferably “in vitro generated antibody constructs”. This term refers to an antibody construct according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. It is also contemplated that the antibody constructs of the present invention are or are based on recombinant antibodies. A “recombinant antibody” is an antibody made through the use of recombinant DNA technology or genetic engineering.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256:495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target cell surface antigen, (Schier, Human Antibodies Hybridomas 7 (1996), 97-105; Malmborg, J. Immunol. Methods 183 (1995), 7-13). Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al, Nature, 352:624-628 (1991) and Marks et al., J. Mol. Biol., 222:581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antibody constructs involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human target cell surface antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antibody constructs of the present disclosure specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antibody construct, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14:14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antibody constructs such as antibody constructs of the present invention, variants or fragments thereof (such as Fv, Fab, Fab′, F(ab′)₂ or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody”, “human antibody construct” and “human binding domain” includes antibodies, antibody constructs such as antibody constructs of the present invention and binding domains having antibody regions such as variable and constant regions or domains which correspond substantially to human germline immunoglobulin sequences known in the art, including, for example, those described by Kabat et al. (1991) (loc. cit.). The human antibodies, antibody constructs or binding domains as defined in the context of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antibody constructs defined herein are “isolated” or “substantially pure” antibody constructs. “Isolated” or “substantially pure”, when used to describe the antibody constructs disclosed herein, means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antibody constructs may e.g., constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antibody construct in a wide variety of organisms and/or host cells that are known in the art. In preferred embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody construct will be prepared by at least one purification step.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” or “polypeptide chain” as used herein describes a group of molecules, which usually consist of more than 30 amino acids. The terms “peptide”, “polypeptide” and “protein” also refer to naturally modified peptides/polypeptides/proteins wherein the modification is affected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below. The above modifications (glycosylation, pegylation etc.) also apply to the antibody constructs of the invention.

Preferably, the binding domain which binds to an antigen on the surface of an innate immune effector cell, the binding domain which binds to an antigen on the surface of a target cell, and/or the binding domain which binds to another antigen on the surface of a target cell is/are human binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs are expected to minimize the immunogenic and allergic responses intrinsic to mouse or mouse-derivatized mAbs and thus to increase the efficacy and safety of the administered antibodies/antibody constructs. The use of fully human antibodies or antibody constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antibody constructs comprising a human binding domain against the antigen on the surface of an innate immune effector cell and/or a human binding domain against the antigen on the surface of a target cell in order to vitiate concerns and/or effects of HAMA or HACA response.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction site”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically, a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

The interaction between the binding domain and the epitope or the region comprising the epitope implies that a binding domain exhibits appreciable affinity for the epitope/the region comprising the epitope on a particular protein or antigen (here: e.g. an antigen on the surface of an innate immune effector cell, such as CD16A, an antigen on the surface of a target cell, and/or another antigen on the surface of a target cell, respectively) and, generally, does not exhibit significant reactivity with proteins or antigens other than e.g. the surface of an innate immune effector cell, the antigen on the surface of a target cell, and/or the other antigen on the surface of a target cell. “Appreciable affinity” includes binding with an affinity of about 10⁻⁶ M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than e.g., the surface of an innate immune effector cell, the antigen on the surface of a target cell, and/or the other antigen on the surface of a target cell.

The term “does not essentially/substantially bind” or “is not capable of binding” means that a binding domain of the present invention does not bind a protein or antigen other than e.g. the e.g. the antigen on the surface of an innate immune effector cell, the antigen on the surface of a target cell, and/or the other antigen on the surface of a target cell, i.e., does not show reactivity of more than 30%, preferably not more than 20%, more preferably not more than 10%, particularly preferably not more than 9%, 8%, 7%, 6% or 5% with proteins or antigens other than e.g. the antigen on the surface of an innate immune effector cell, the antigen on the surface of a target cell, and/or the other antigen on the surface of a target cell, whereby binding to e.g. the antigen on the surface of an innate immune effector cell, the antigen on the surface of a target cell, and/or the other antigen on the surface of a target cell, respectively, is set to be 100%.

Specific binding is believed to be affected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding side.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit; Chothia et al., J. Mol. Biol, 1987, 196:901-917; and MacCallum et al., J Mol. Biol, 1996, 262:732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196:901; Chothia et al., Nature, 1989, 342:877; Martin and Thornton, J. Mol. Biol, 1996, 263:800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. A global reference in immunoinformatics is the three-dimensional (3D) structure database of IMGT (international ImMunoGenetics information system) (Ehrenmann et al., 2010, Nucleic Acids Res., 38, D301-307). The IMGT/3D structure-DB structural data are extracted from the Protein Data Bank (PDB) and annotated according to the IMGT concepts of classification, using internal tools. Thus, IMGT/3D structure-DB provides the closest genes and alleles that are expressed in the amino acid sequences of the 3D structures, by aligning these sequences with the IMGT domain reference directory. This directory contains, for the antigen receptors, amino acid sequences of the domains encoded by the constant genes and the translation of the germline variable and joining genes. The CDR regions of our amino acid sequences were preferably determined by using the IMGT/3D structure database.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2nd ed., eds. Jonio et al., Academic Press, San Diego, CA, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The antibody construct defined in the context of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. Strepll-tag) and His-tag. All herein disclosed antibody constructs characterized by the identified CDRs may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antibody construct, preferably it is located at the C-terminus. Most preferably, a hexa-histidine tag is linked via peptide bond to the C-terminus of the antibody construct according to the invention. Additionally, a conjugate system of PLGA-PEG-PLGA may be combined with a poly-histidine tag for sustained release application and improved pharmacokinetic profile.

Amino acid sequence modifications of the antibody constructs described herein are also contemplated. For example, it may be desirable to improve the binding affinity and/or other biological properties of the antibody construct. Amino acid sequence variants of the antibody constructs are prepared by introducing appropriate nucleotide changes into the antibody constructs nucleic acid, or by peptide synthesis. All of the below described amino acid sequence modifications should result in an antibody construct which still retains the desired biological activity (e.g. the antigen on the surface of an innate immune effector cell, the antigen on the surface of a target cell, and/or the other antigen on the surface of a target cell) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or F); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, Ile, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also be performed within a third binding domain of the antibody construct defined in the context of the invention. An insertional variant of the antibody construct defined in the context of the invention includes the fusion to the N-terminus or to the C-terminus of the antibody construct of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antibody constructs that are preferred locations for mutagenesis is called “alanine scanning mutagenesis” as described by Cunningham and Wells in Science, 244: 1081-1085 (1989). Here, a residue or group of target residues within the antibody construct is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as for the binding to e.g. an antigen on the surface of an innate immune effector cell, an antigen on the surface of a target cell, and/or another antigen on the surface of a target cell.

Generally, if amino acids are substituted in one or more or all of the CDRs of the heavy and/or light chain, it is preferred that the then-obtained “substituted” sequence is at least 60% or at least 65%, more preferably at least 70% or at least 75%, even more preferably at least 80% or at least 85%, and particularly preferably at least 90% or at least 95% identical to the “original” CDR sequence. This means that it is dependent of the length of the CDR to which degree it is identical to the “substituted” sequence. For example, a CDR having 5 amino acids is preferably at least 80% identical to its substituted sequence in order to have at least one amino acid substituted. Accordingly, the CDRs of the antibody construct may have different degrees of identity to their substituted sequences, e.g., CDRL1 may have at least 80%, while CDRL3 may have at least 90%.

Preferred substitutions (or replacements) are conservative substitutions. However, any substitution (including non-conservative substitution or one or more from the “exemplary substitutions” listed in Table 3, below) is envisaged as long as the antibody construct retains its capability to bind to e.g. the antigen on the surface of an innate immune effector cell via the first binding domain (A), to the antigen on the surface of a target cell via the second binding domain (B), and/or to the other antigen on the surface of a target cell via an optional third binding domain (C) and/or its CDRs have an identity to the then substituted sequence (at least 60% or at least 65%, more preferably at least 70% or at least 75%, even more preferably at least 80% or at least 85%, and particularly preferably at least 90% or at least 95% identical to the “original” CDR sequence).

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened for a desired characteristic.

TABLE 1
Amino acid substitutions

| Original | Exemplary Substitutions | Preferred Substitutions |
|---|---|---|
| Ala (A) | val, leu, ile | val |
| Arg (R) | lys, gln, asn | lys |
| Asn (N) | gln, his, asp, lys, arg | gln |
| Asp (D) | glu, asn | glu |
| Cys (C) | ser, ala | ser |
| Gln (Q) | asn, glu | asn |
| Glu (E) | asp, gln | asp |
| Gly (G) | ala | ala |
| His (H) | asn, gln, lys, arg | arg |
| Ile(I) | leu, val, met, ala, phe | leu |
| Leu (L) | norleucine, ile, val, met, ala | lie |
| Lys (K) | arg, gln, asn | arg |
| Met (M) | leu, phe, ile | leu |
| Phe (F) | leu, val, ile, ala, tyr | tyr |
| Pro (P) | ala | ala |
| Ser (S) | thr | thr |
| Thr (T) | ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antibody construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VI, defined in the context of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antibody constructs according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antibody constructs leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antibody constructs. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

The term “EGFR” refers to the epidermal growth factor receptor (EGFR; ErbB-1: HER1 in humans, including all isoforms or variants described with activation, mutations and implicated in pathophysiological processes. The EGFR antigen-binding site recognizes an epitope in the extracellular domain of the EGFR. In certain embodiments the antigen-binding site specifically binds to human and cynomolgus EGFR. The epidermal growth factor receptor (EGFR) is a member of the HER family of receptor tyrosine kinases and consists of four members: EGFR (ErbB1/HER1), HER2/neu (ErbB2), HER3 (ErbB3) and HER4 (ErbB4). Stimulation of the receptor through ligand binding (e.g. EGF, TGFa, HB-FGF, neuregulins, betacellulin, amphiregulin) activates the intrinsic receptor tyrosine kinase in the intracellular domain through tyrosine phosphorylation and promotes receptor homo- or heterodimerization with HER family members. These intracellular phospho-tyrosines serve as docking sites for various adaptor proteins or enzymes including SHC, GRB2, PLCg and PI(3)K/Akt, which simultaneously initiate many signaling cascades that influence cell proliferation, angiogenesis, apoptosis resistance, invasion and metastasis.

The term “immune effector cell” as used herein may refer to any leukocyte or precursor involved e.g. in defending the body against cancer, diseases induced by infectious agents, foreign materials or autoimmune reactions. For example, the immune effector cells comprise B lymphocytes (B cells), T lymphocytes (T cells, including CD4+ and CD8+ T cells), NK cells, NKT cells, monocytes, macrophages, dendritic cells, mast cells, granulocytes such as neutrophils, basophils and eosinophils, innate lymphoid cells (ILCs, which comprise ILC-1, ILC-2 and ILC-3), γδ T cells or any combinations thereof. The term “innate immune effector cell” refers to immune effector cells that belong to the innate immune system. For example, innate immune effector cells comprise natural, modified or engineered NK cells, monocytes, macrophages, dendritic cells, mast cells, granulocytes such as neutrophils, basophils and eosinophils, innate lymphoid cells (ILCs, which comprise ILC-1, ILC-2 and ILC-3), induced pluripotent stem cell (iPSC)-derived innate immune cells (e.g. iPSC-NK cells, iPSC-macrophages), engineered immune effector cells (e.g. T cells) expressing immune-regulatory receptors (e.g. CD16A, NKp46) or any combinations thereof. Preferably, the term innate immune effector cell refers to an NK cell and/or a macrophage.

Natural killer (NK) cells are CD56+CD3-large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+T lymphocytes, NK cells launch cytotoxicity against tumor cells without the requirement for prior sensitization and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

Monocytes are produced by the bone marrow from haematopoietic stem cell precursors called monoblasts. Monocytes circulate in the bloodstream for about one to three days and then typically move into tissues throughout the body. They constitute between three to eight percent of the leukocytes in the blood. In the tissue monocytes mature into different types of macrophages at different anatomical locations. Monocytes have two main functions in the immune system: (1) replenish resident macrophages and dendritic cells under normal states, and (2) in response to inflammation signals, monocytes can move quickly (approx. 8-12 hours) to sites of infection in the tissues and divide/differentiate into macrophages and dendritic cells to elicit an immune response. Monocytes are usually identified in stained smears by their large bilobate nucleus.

Macrophages are potent effectors of the innate immune system and are capable of at least three distinct anti-tumor functions: phagocytosis, cellular cytotoxicity, and antigen presentation to orchestrate an adaptive immune response. While T cells require antigen-dependent activation via the T cell receptor or the chimeric immunoreceptor, macrophages can be activated in a variety of ways. Direct macrophage activation is antigen-independent, relying on mechanisms such as pathogen associated molecular pattern recognition by Toll-like receptors (TLRs). Immune-complex mediated activation is antigen dependent but requires the presence of antigen-specific antibodies and absence of the inhibitory CD47-SIRPa interaction.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, TH9, or TFH, which secrete different cytokines to facilitate a different type of immune response.

Cytotoxic T cells (TC cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described naturally occurring Treg cells and adaptive Treg cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

As used herein, the term “engineered immune effector cell” relates to genetically modified immune cells by a method or a tool which allows for gene editing. Examples of methods for immune cell engineering include but limited to viral transduction, zinc-finger nucleases, transcription activator-like effector nucleases (TALEN) and CRISPR/Cas9. Engineered immune cells may also include adaptive immune cells (e.g. T cells) gaining innate immune functions by e.g. expressing immune regulatory receptors normally expressed by innate immune cells (e.g. CD16A. NKp46) (Quelle: (CD16A CAR T cells D'Aloia et al Chimeric Antigen Receptor T Cells, 18 (2): 278-290).

As used herein, the term “half-life extensions domain” relates to a moiety that prolongs serum half-life of the antibody construct. The half-life extension domain may comprise a portion of an antibody, such as an Fc part of an immunoglobulin, a hinge domain, a CH2 domain, a CH3 domain, and/or a CH4 domain. Although less preferred, a half-life extension domain can also comprise elements that are not comprised in an antibody, such as an albumin binding peptide, an albumin binding protein, or transferrin to name only a few. A half-life extension domain preferably does not have an immune-modulatory function. If a half-life extension domain comprises a hinge, CH2 and/or CH3 domain, the half-life extension domain preferably does not essentially bind to an Fc receptor. This can e.g. be achieved through “silencing” of the Fcγ receptor binding domain.

As used herein, “silencing” of the Fc or Fcγ receptor binding domain refers to any modification that reduces binding of a CH2 domain to an Fc receptor, in particular an Fcγ receptor. Such modification can be done by replacement and/or deletion of one or more amino acids that are involved in Fc(γ) receptor-binding. Such mutations are well known in the art and have e.g. been described by Saunders (2019, Front. Immunol. 10:1296). For example, a mutation can be located at any one of the positions 233, 234, 235, 236, 237, 239, 263, 265, 267, 273, 297, 329, and 331. Examples for such mutations are: deletion of Glu 233->Pro, Glu 233, Leu 234->Phe, Leu 234->Ala, Leu 234->Gly, Leu 234->Glu, Leu 234->Val, deletion of Leu 234, Leu 235->Glu, Leu 235->Ala, Leu 235->Arg, Leu 235->Phe, deletion of Leu 235, deletion of Gly 236, Gly 237->Ala, Ser 239->Lys, Val 263->Leu, Asp 265->Ala, Ser 267->Lys, Val 273->Glu, Asn 297->Gly, Asn 297->Ala, Lys 332->Ala, Pro 329->Gly, Pro 331->Ser and combinations thereof. Preferably, such a modification comprises one or both of Leu 234->Ala and Leu 235->Ala (also known as “LALA” mutation). Preferably, such a modification further comprises a Pro 329->Gly mutation, also known as “LALA-PG” mutation (Leu 234->Ala, Leu 235->Ala, and Pro 329->Gly). Preferably, such a modification comprises 1, 2, or 3 of the mutations Leu 234->Phe, Leu 235->Glu, and Asp 265->Ala, more preferably all three of these mutations. The combination Leu 234->Phe, Leu 235->Glu, and Asp 265->Ala, which is a preferred modification in the context of the present invention, is also known as “FEA” mutation. Preferably, such a modification further comprises Asn 297->Gly. Such a preferred modification comprises the mutations Leu 234->Phe, Leu 235->Glu, Asp 265->Ala, and Asn 297->Gly.

The term “fratricide” describes in the context of the invention the reduction of effector cells by cytotoxic kill and, thereby the reduction of the available effector cell population/compartment. Fratricide can be caused by cross-linking of two immune cells. As an illustrative example, cross-linking of NK cells can cause the killing of either one or both of the NK cells. In cases where an antibody construct recruits two different types of effector cells, e.g. NK cells and macrophages or NK cells and T cells also the elimination of one type of effector cells by the other type of effector cells is understood as fratricide. Fratricide can be e.g. measured in an assay as essentially described in Example 7.

### DETAILED DESCRIPTION

The present invention is based on the finding that tumor mass is highly heterogeneous with respect to expression level of given tumor antigen (high, medium, low). In the course of antitumoral treatment, tumors might downregulate targeted tumor antigen during therapy as an escape strategy. Tumor cells low for targeted tumor antigen might account for potential to relapse after initial CR. This is believed to be a general challenge for all strategies targeting tumor antigens.

For the application of bi- or multispecific immune effector cell engagers, downregulation of targeted tumor antigen will reduce the number of engaged signaling competent tumor antigen/immune cell antigen complexes below the threshold required for efficient killing by innate immune receptor cells, such as NK cell. However, it was surprisingly found by the inventors of the present application that improved killing of tumor cells low for targeted tumor antigen might be achieved by increasing the number of binding domains for an immune-regulatory antigen of an immune effector cell per engaged individual tumor antigen.

Thus, the present invention provides bispecific or multispecific immune effector cell engager format with four valences for an immune regulatory antigen of an innate immune effector cell, to increase activation threshold and affinity/cell surface retention for the innate immune effector cell. By increasing the number of binding domains for the innate immune effector cell antigen, one tumor antigen can induce activation via up to four innate immune effector cell antigens, such as CD16A.

The use of engager comprising at least four binding domains for the immune-regulatory antigen of the innate immune cell may mediate long lasting maximal efficacy of innate immune cells, such as NK cells and/or macrophages. Further killing kinetics of such engagers can be faster when compared to conventional engagers having less than four binding domains for the immune-regulatory antigen of the innate immune cell (such as one or two). Bi- or multi-specific engagers having at least four binding domains for the immune-regulatory antigen of the innate immune cell can also display deeper response and thus lead to full eradication of target-positive tumor cells, which is especially interesting for combination with a dual-targeting approach, where the engager is specific for at least two antigens on the surface of a target cell. Thus, bi- and multi-specific engagers of the present invention might be the preferred format for targeting tumor antigens with very low abundance (including peptide/MHC-I complexes).

Thus, antibody constructs of the invention can effectively target tumor cells with downregulated or naturally low expression of the target antigen, which makes the antibody constructs particularly useful for the treatment of natural tumors with heterogenous expression of the tumor antigen, since the antibody construct can target the entire tumor. Further, by killing low expressing cells, e.g. tumor stem cells, the antibody constructs can also prevent relapse of the tumor.

The present invention thus envisions an antibody construct comprising (i.) at least four first binding domains (A), wherein said first binding domain (A) is capable of specifically binding to a first target (A′) that is an antigen on the surface of an innate immune effector cell; and (ii.) a second binding domain (B), which is capable of specifically binding to a second target (B′) that is an antigen on the surface of a target cell. The antigen on the surface of the innate immune cell is preferably an immune-regulatory antigen. The innate immune cell is preferably a natural killer cell or a macrophage.

With regard to the first binding domains (A), the term “at least four” includes 5, 6, 7, 8, or even higher numbers. However, 4, 5, and 6 are preferred, with 4 being most preferred. Similarly, with regard to the second binding domain (B), the antibody construct of the disclosure can comprise more than one second binding domain, such as 2, 3, 4 or even more. However, it is preferred that the antibody construct of the present invention comprises 1 or 2 second binding domains (B), with 2 being most preferred. It is understood that the second target (B′) is on the surface of a target cell that is not the same cell as the innate immune effector cell, which expresses the first target (A′)

The antibody construct of the present invention may be capable of binding to a target cell and an (innate) immune effector cell simultaneously. Binding to the (innate) immune effector cell can be via at least one of the at least four first binding domains (A). Binding to the target cell can be via at least one second binding domain (B) and/or at least one optional third binding domain (C). The antibody constructs of the disclosure may be bispecific. The antibody constructs of the disclosure may also be trispecific. Bispecific antibody constructs are however preferred.

Binding of one or more of the at least four first binding domains (A) to the first target (A′) might boost the functionality of immune effector cells by inducing activation signals or blocking inhibitory signals, which is referred herein as “immune-regulatory antigen”, in particular on NK cells and/or macrophages Such “immune-regulatory antigen” includes but is not limited to CD16A, CD56, NKG2A, NKG2D, NKp30, NKp44, NKp46, NKp80, DNAM-1 (CD226), SLAMF7 (CD319), CD244 (2B4), OX40, CD47, SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5, KIR3DS1, and CD3. Moreover, the first target (A′) is an immune-regulatory antigen that can be grouped into different categories depending on the mechanism of action: (1) Antigens inducing an activation of the immune effector cell, which comprise, but not limited to, CD16A, CD56, NKG2D, NKp30, NKp44, NKp46, NKp80, DNAM-1 (CD226), SLAMF7 (CD319), CD244 (2B4), OX40, CD137, CD89, CD160, and killer-cell immunoglobulin-like receptors (KIR2DS1-5 and KIR3DS1), which are referred to herein as “immune activating antigen(s)”. (2) inhibitory antigens on effector cells comprising e.g. NKG2A, TIGIT, PD-1, PD-L1, CD47, SIRPα, LAG-3, CTLA-4, CD96, TIM-3, CD137, KIR2DL1-5 and KIR3DL1-3, which are referred to herein as “immune inhibitory antigen(s)”, which may be blocked to counteract inhibition and/or functional exhaustion. A first binding partner (A) for an “immune activating antigen” is preferably an agonist. A first binding partner (A) for an “immune inhibitory antigen” is preferably an antagonist.

The antigens inducing activation of the effector cells can be additionally classified in groups according the signaling cascade: (1) CD32-dependent/CD16A-associated signaling such as CD16A, NKp46, NKp30 and (2) CD3-independent signaling such as, but not limited to, NKG2D, NKp44, NKp80, DNAM-1 (CD226), SLAMF7 (CD319), CD244 (2B4) and killer-cell immunoglobulin-like receptors (e.g. KIR2DS1).

Depending on the selection of the antigen for the at least four first binding domains (A), different cell types will be potentially targeted/activated such as, but not limited to, NK cells with antigens selected from the group comprising e.g. CD16A, CD56, NKG2D, NKp30, NKp44, NKp46, NKp80, DNAM-1 (CD226), SLAMF7 (CD319), CD244 (2B4), OX40, CD137, CD160, KIR2DS1-5, NKG2A, TIGIT, PD-1, PD-L1, CD47, LAG-3, CTLA-4, CD96, TIM-3, CD137, KIR2DL1-5 and KIR3DL1-3; and/or monocytes, macrophages and/or neutrophils with antigens selected from the group comprising e.g. CD16A, CD89, SLAMF7, SIRPα, and/or CD47. Moreover, dependent on the antigen, different subpopulations (e.g. CD56^{dim}CD16^{bright} NK cells, CD56^{bright}CD16^{negative} NK cells, peripheral or tissue resident NK cells, M1 or M2 macrophages, tumor-associated macrophages) can be addressed.

In some embodiments, the at least four first binding domains (A) are specific for a (CD) antigen that is preferably selected from the group consisting of CD16A, CD56, NKG2A, NKG2D, NKp30, NKp44, NKp46, NKp80, DNAM-1 (CD226), SLAMF7 (CD319), CD244 (2B4), OX40, CD47, SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5, KIR3DS1, and CD3.

According to the present disclosure, the first target (A′) is preferably selected from the group consisting of CD16A, CD56, NKG2A, NKG2D, NKp30, NKp44, NKp46, NKp80, DNAM-1 (CD226), SLAMF7 (CD319), CD244 (2B4), OX40, CD47, SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5, KIR3DS1, and CD3, with CD16A and NKp46 being preferred, with CD16A being most preferred. In this context, preferred first targets (A′) are targets that are present on NK cells and/or macrophages. The at least four first binding domains (A) can bind to the same epitope of the first target (A′).

The antibody construct of the disclosure may comprise a third binding domain (C), which is capable of specifically binding to a third target (C′). The third target (C′) is an antigen on the surface of a target cell that is other than the second target (B′). However, the antibody construct of the disclosure can comprise more than one third binding domain (C), such as 2, 3, 4 or even more. However, it is preferred that the antibody construct of the disclosure comprises 1 or 2 third binding domains (B). Preferred antibody constructs of the disclosure may thus comprise one second binding domain (B) and one third binding domain (C), two second binding domains (B) and one third binding domain (C), one second binding domain (B) and two third binding domains (C), or two second binding domains (B) and two third binding domains (C), with one second binding domain (B) and one third binding domain (C) being most preferred.

The first binding domain (A) is preferably derived from an antibody. The first binding domain (A) preferably comprises a VH and a VL domain of an antibody. Exemplary structures for the first binding domain (A) include an Fv, an scFv, a Fab, or a VL and VH pair which may be comprised in a diabody (Db), scDb, a bi-scFv or a double Fab, with a VL and VH pair comprised in a scDb or bi-scFv being preferred.

The second binding domain (B) is also preferably derived from an antibody. The second binding domain (B) preferably comprises a VH and a VL domain of an antibody. Exemplary structures for the second binding domain (B) include an Fv, an scFv, a Fab, or a VL and VH pair which may be comprised in a diabody (Db), scDb or a double Fab, with an scFv or Fab being preferred.

The third binding domain (C) is also preferably derived from an antibody. The third binding domain (C) preferably comprises a VH and a VL domain of an antibody. Exemplary structures for the third binding domain (C) include an Fv, an scFv, a Fab, or a VI, and VH pair which may be comprised in a diabody (Db), scDb or a double Fab, with an scFv or Fab being preferred.

The antibody construct of the disclosure may comprise a fourth domain (D), which comprises a half-life extension domain as described herein. The half-life extension domain may comprise a CH2 domain, in which the Fcγ receptor binding domain of the CH2 domain is silenced. The half-life extension domain may comprise two such CH2 domains. Whenever a half-life extension domain comprises a CH2 domain, the Fcγ receptor binding domain of the CH2 domain is silenced. The half-life extension domain may comprise a CH3 domain. The half-life extension domain may comprise two CH3 domains. The half-life extension domain may comprise a hinge domain. The half-life extension domain may comprise two hinge domains. The half-life extension domain may comprise a CH2 domain and a CH3 domain. In such a case, the CH2 domain and CH3 domain are preferably fused to each other, preferably in the (amino to carboxyl) order CH2 domain-CH3 domain. Non-limiting examples for such fusions are shown in SEQ ID NOs: 39-58. The half-life extension domain may comprise a hinge domain and a CH2 domain. In such a case, the hinge domain and the CH2 domain are preferably fused to each other, preferably in the (amino to carboxyl) order hinge domain-CH2 domain. The half-life extension domain may comprise a hinge domain, a CH2 domain, and a CH3 domain. In such a case, the hinge domain, the CH2 domain, and CH3 domain are preferably fused to each other, preferably in the (amino to carboxyl) order hinge domain-CH2 domain-CH3 domain. The half-life extending domain may comprise two hinge domain CH2 domain elements, two CH2 domain-CH3 domain elements, or two hinge domain-CH2 domain-CH3 domain elements. In such a case the two fusions may be located on two different polypeptide strands. Alternatively, the fusions can be located on the same polypeptide strand. An illustrative example for two hinge domain-CH2 domain-CH3 domain elements that are located on the same polypeptide strand is the “single chain Fc” or “scFc” format. Here, both hinge-CH2-CH3 subunits are fused together via a linker that allows assembly of a Fc domain. A preferred linker for this purpose is a glycine serine linker, which preferably comprises from about 20 to about 40 amino acids. Preferred glycine serine linkers may have one or more repeats of GGS, GGGS (SEQ ID NO: 1), or GGGGS (SEQ ID NO: 6). Such linker preferably comprises 4-8 repeats (e.g. 4, 5, 6, 7, or 8 repeats) of GGGGS. Such a linker is preferably (GGGGS) 6, (SEQ ID NO 9). Illustrative examples for such scFc domains are shown in SEQ ID NOs 2-5. Further scFc constant domains are known in the art and inter alia described in WO 2017/134140.

Generally, with regard to the second target (B′) and/or optionally the third target (C′), the antibody constructs of the disclosure can be monovalent, bivalent, trivalent, or have an even higher valency for any one of the second target (B′) and/or optionally the third target (C′). For the first target (A′), the antibody constructs of the disclosure can be tetravalent or have an even higher valency. The antibody construct of the disclosure may thus comprise four, five, six, or even more of any one of the first binding domain (A). The antibody construct of the disclosure can comprise one, two, three, or even more of the second binding domain (B). Optionally, the antibody construct of the disclosure can comprise one, two, three, or even more of the third binding domain (C). It is preferred for the antibody construct of the disclosure that it is at least tetravalent for the first target (A′) and at least monovalent or at least bivalent for the second target (B′). It is further preferred for the antibody construct of the disclosure that it is at least tetravalent for the first target (A′), at least monovalent for the second target (B′), and at least monovalent for the third target (C′). More preferably, the antibody construct of the disclosure is tetravalent for the first target (A′) and monovalent for the second target (B′). More preferably, the antibody construct of the disclosure is tetravalent for the first target (A′), monovalent for the second target (B′), and monovalent for the third target (C′). Even more preferably, the antibody construct of the disclosure is tetravalent for the first target (A′) and bivalent for the second target (B′). It is preferred for the antibody construct of the disclosure that it comprises at least two four first binding domains (A) and at least one or at least two second binding domains (B). It is further preferred for the antibody construct of the disclosure that it comprises at least four first binding domains (A), at least one second binding domain (B), and at least one third binding domain (C). More preferably, the antibody construct of the disclosure comprises four first binding domains (A) and one second binding domain (B). More preferably, the antibody construct of the invention comprises four first binding domains (A), one second binding domain (B), and one third binding domain (C). Even more preferably, the antibody construct of the invention comprises four first binding domains (A) and two second binding domains (B).

In order to reduce immune effector cell fratricide, it is also envisaged that the four first binding domains (A) should be positioned to each other in a way that simultaneous binding of two immune effector cells is reduced or preferably prevented. This can e.g., be achieved by providing a short distance between the first binding domains (A). For example, a first first binding domain (A1) and a second first binding domain (A2) may be fused to each other to form a pair (A1A2), also referred to herein as dimer. Such a dimer (A1A2) may be in form of a bi-scFv, a double fab, a Db or scDb. It is however preferred that such a dimer (A1A2) is in the form of a bi-scFv or scDb. The spatial arrangement of the variable domains of a bi-scFv can be in any suitable order, with a VH-VL-VH-VL order being preferred. The spatial arrangement of the variable domains of an scDb can be in any suitable order, with a VL-VH-VL-VH order being preferred. The most preferred format for a dimer of two first binding domains (A1A2) is the scDb format. In such an scDb, the domains on the polypeptide on the polypeptide chain are preferably arranged in the (N to C) order VL-VH-VL-VH.

Similarly, also a third first binding domain (A3) and a fourth binding domain (A4) can be fused to each other to form a pair (A3A4), also referred to as dimer. Again, such a dimer (A3A4) may be in form of a bi-scFv, a double Fab, a Db or scDb. It is however preferred that such a dimer (A3A4) is in the form of a bi-scFv or scDb. The spatial arrangement of the variable domains of a bi-scFv can be in any suitable order, with a VH-VL-VH-VL order being preferred. The spatial arrangement of the variable domains of an scDb can be in any suitable order, with a VL-VH-VL-VH order being preferred. The most preferred format for a dimer of two first binding domains (A3A4) is the scDb format. In such a scDb, the domains on the polypeptide on the polypeptide chain are preferably arranged in the (N to C) order VL-VH-VL-VH.

The at least four first binding domains (A) are preferably fused to the fourth domain. The first binding domains can be arranged as monomers, but it is preferred that the first binding domains are in the form of at least two dimers ((A1A2) and (A3A4)). The preferred fourth binding domain comprises two hinge domain-CH2 domain-CH3 domain elements, which can be in form of a scFc, or preferably in form of a Fc. Generally, the four first binding domains (A) can be fused to any N or C terminus of the scFc, or preferably the Fc. In a preferred arrangement, a first binding domain and a second first binding domain that are fused to each other (A1A2) is fused to the C terminus of a CH3 domain of a fourth domain (D). In a preferred arrangement, a first binding domain and a second first binding domain that are fused to each other (A1A2) is fused to the N terminus of a hinge of a fourth domain (D). In a preferred arrangement, a third binding domain and a fourth first binding domain that are fused to each other (A3A4) is fused to the C terminus of a CH3 domain of a fourth domain (D). In a preferred arrangement, a third binding domain and a fourth first binding domain that are fused to each other (A3A4) is fused to the N terminus of a hinge of a fourth domain (D).

In the antibody constructs of the disclosure, a first first binding domain and a second first binding domain that are fused to each other (A1A2) can be fused to the C terminus of a CH3 domain of a fourth domain (D), whereas a third first binding domain and a fourth first binding domain that are fused to each other (A3A4) is fused to the N terminus of a hinge of a fourth domain (D). However, in order to provide shorter distances between the first binding domains, it is preferred that both dimers of the first binding domain, (A1A2) and (A3A4) are either fused to two N termini of an Fc of the fourth domain (D), or, more preferably, to two C termini of an Fc of the fourth domain (D). Therefore, in a preferred antibody construct of the disclosure, a first first binding domain and a second first binding domain that are fused to each other (A1A2) are fused to the N terminus of a first hinge domain of a fourth domain (D), whereas a third first binding domain and a fourth first binding domain that are fused to each other (A3A4) are fused to the N terminus of a second hinge domain of a fourth domain (D). In an even more preferred antibody construct of the disclosure, a first first binding domain and a second first binding domain that are fused to each other (A1A2) are fused to the C terminus of a first CH3 domain of a fourth domain (D), whereas a third first binding domain and a fourth first binding domain that are fused to each other (A3A4) are fused to the C terminus of a second CH3 domain of a fourth domain (D).

In the antibody constructs of the disclosure, a second binding domain (B) can be fused to the fourth domain (D). Generally, the second binding domain (B) can be fused at any position suitable for such a fusion, in particular at any N or C terminus of the fourth domain (D). Accordingly, the second binding domain (B) can be fused to the N terminus of a hinge domain of the fourth domain (D). The second binding domain can also be fused to the C terminus of a CH3 domain of the fourth domain (D).

In cases where the antibody construct comprises at least two second binding domains (B), the at least two second binding domains can be individually fused at any position suitable for such a fusion, in particular at any N or C terminus of the fourth domain (D). For example, one second binding domain (B) can be fused to the N terminus of a hinge domain of a fourth domain (D), whereas another second binding domain (B) is fused to the C terminus of a CH3 domain of a fourth domain (D). However, arrangements where the two second binding domains are both fused to the N termini of the fourth domain (D) or both fused to the C termini of the fourth domain (D) are preferred. Thus, it is preferred that a second binding domain (B) is fused to the N terminus of a hinge of a fourth domain (D), while another second binding domain (B) is fused to the N terminus of another hinge of the fourth domain (D). It is also preferred that a second binding domain (B) is fused to the C terminus of a CH3 of a fourth domain (D), while another second binding domain (B) is fused to the C terminus of another CH3 of the fourth domain (D).

In antibody constructs comprising a third binding domain (C), the third binding domain (C) can be fused to the fourth domain (D). Generally, the third binding domain (C) can be fused at any position suitable for such a fusion, in particular at any N or C terminus of the fourth domain (D). Accordingly, the third binding domain (C) can be fused to the N terminus of a hinge domain of the fourth domain (D). The third binding domain can also be fused to the C terminus of a CH3 domain of the fourth domain (D).

In the antibody constructs of the disclosure, a second binding domain (B) can be fused to the N terminus of a hinge domain of a fourth domain (D), whereas a third binding domain (C) is fused to the C terminus of a CH3 domain of a fourth domain (D), or vice versa. However, arrangements where the second binding domain (B) and the third binding domain (C) are both fused to the N termini of the fourth domain (D) or both fused to the C termini of the fourth domain (D) are preferred. Thus, it is preferred that a second binding domain (B) is fused to the N terminus of a hinge of a fourth domain (D), while a third binding domain (C) is fused to the N terminus of another hinge of the fourth domain (D). It is also preferred that a second binding domain (B) is fused to the C terminus of a CH3 of a fourth domain (D), while a third binding domain (C) is fused to the C terminus of another CH3 of the fourth domain (D).

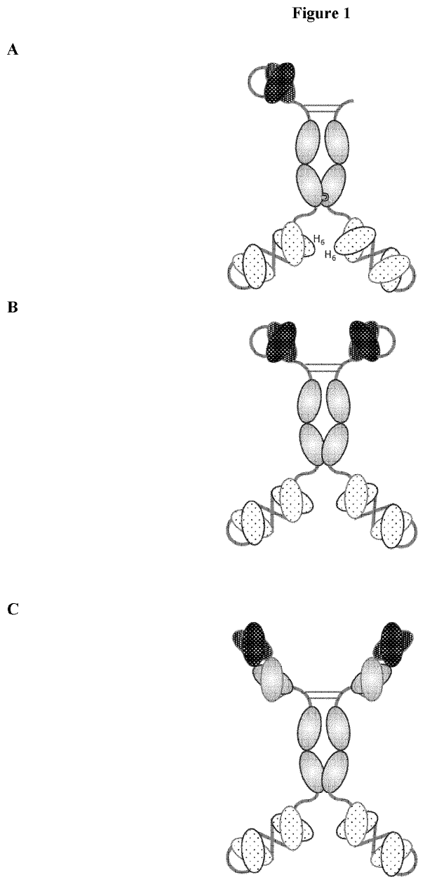

In a preferred antibody construct, a first dimer of two first binding domains (A1A2) and a second dimer of two first binding domains (A3A4) are fused to two C termini of a Fc region. Such a fusion format is illustratively shown in FIG. 1A-C. In the first dimer (A1A2), the two first binding domains (A1 and A2) are preferably fused together in form of a diabody or single chain diabody, preferably via a VL domain of a first first binding domain (A1). Likewise, in the second dimer (A3A4), the two first binding domains (A3 and A4) are preferably fused together in form of a diabody or single chain diabody, preferably via a VL domain of a third first binding domain (A3). The first dimer (A1A2) and/or second dimer (A3A4) may be fused to a constant domain of an antibody via a linker. Such a linker is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or preferably even less. The length of the linker is preferably determined as described by Rossmalen et al Biochemistry 2017, 56, 6565-6574, which also describes suitable linkers that are well known to the skilled person. An example for a suitable linker is a glycine serine linker or a serine linker, which preferably comprises not more than about 75 amino acids, preferably not more than about 50 amino acids. In illustrative examples, a suitable linker comprises one or more GGGGS sequences (SEQ ID NO: 6), such as (GGGGS)₂ (SEQ ID NO: 7), (GGGGS)₄ (SEQ ID NO: 8), or preferably (GGGGS)₆ (SEQ ID NO: 9). Other illustrative examples for linkers are shown in SEQ ID NOs: 2-5. The first dimer (A1A2) and/or the second dimer (A3A4) are preferably scDb fragments that are fused to two C termini of a Fc domain, preferably via a VL domain of the scDb. Accordingly, the arrangement of on the polypeptide chain (from N to C) is preferably . . . —CH2-CH3-VL-VH-VL-VH, optionally with a linker between the Fc and the scDb. One or two second binding domain (B) can be located at any suitable position of the antibody construct. Similarly, a third binding domain (C) can also be located at any suitable position of the antibody construct. Where the antibody construct comprises a Fc region, a second binding domain (B) and/or a third binding domain (C) can be located N terminal of the Fc region, either directly or linked via at least a part of a hinge domain. Other linkers disclosed herein can also be used to link the second and/or third binding domain(s) to the Fc domain. A hinge domain is however preferred for this purpose. A second binding domain (B) can be any suitable structure disclosed herein, including Fab and scFv, while an scFv structure is preferred. Similarly, a third binding domain (C) can be any suitable structure disclosed herein, including Fab and scFv, while an scFv structure is preferred. In case of an scFv, the scFv is preferably fused to the Fc domain via the VL region comprised in the scFv.

A preferred antibody construct of the invention is preferably in a format as essentially shown in FIG. 1A. Such an antibody construct comprises an immunoglobulin that has two scDb fragments fused to the C-termini of the heavy chains, optionally via a linker, which is preferably a glycine serine linker or a serine linker, preferably a glycine serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 6), such as (GGGGS)₂ (SEQ ID NO: 7), (GGGGS)₄ (SEQ ID NO: 8), or preferably (GGGGS)₆ (SEQ ID NO: 9). Other illustrative examples for linkers are shown in SEQ ID NOs: 2-5. One of the two scDb comprises two first binding domains (A1 and A2), while the other scDb also comprises two first binding domains (A3 and A4). One second binding domain (B) is fused to one N terminus of one Fc region. Such an antibody construct may comprise two polypeptide chains, one polypeptide chain in the arrangement VH(B)-VL(B)-hinge-CH2-CH3-VL(A2)-VH(A1)-VL(A1)-VH(A2) (or less preferred VH(B)-VL(B)-hinge-CH2-CH3-VH(A2)-VL(A1)-VH(A1)-VL(A2), VL(B)-VH(B)-hinge-CH2-CH3-VL(A2)-VH(A1)-VL(A1)-VH(A2), or VL(B)-VH(B)-hinge-CH2-CH3-VH(A2)-VL(A1)-VH(A1)-VL(A2)), and another polypeptide chain in the arrangement hinge-CH2-CH3-VL(A4)-VH(A3)-VL(A3)-VH(A4) (or less preferred hinge-CH2-CH3-VH(A4)-VL(A3)-VH(A3)-VL(A4)). Illustrative examples for such antibody constructs are shown in SEQ ID NOs: 152-153 and 158-159.

A preferred antibody construct of the invention is preferably in a format as essentially shown in FIG. 1B. Such an antibody construct comprises an immunoglobulin that has two scDb fragments fused to the C-termini of the heavy chains, optionally via a linker, which is preferably a glycine serine linker or a serine linker, preferably a glycine serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 6), such as (GGGGS)₂ (SEQ ID NO: 7), (GGGGS)₄ (SEQ ID NO: 8), or preferably (GGGGS)₆ (SEQ ID NO: 9). Other illustrative examples for linkers are shown in SEQ ID NOs: 2-5. One of the two scDb comprises two first binding domains (A1 and A2), while the other scDb also comprises two first binding domains (A3 and A4). Two second binding domains (B) are fused to the N termini of the Fc regions. Such an antibody construct may comprise two polypeptide chains, one polypeptide chain in the arrangement VH(B)-VL(B)-hinge-CH2-CH3-VL(A2)-VH(A1)-VL(A1)-VH(A2) (or less preferred VH(B)-VL(B)-hinge-CH2-CH3-VH(A2)-VL(A1)-VH(A1)-VL(A2), VL(B)-VH(B)-hinge-CH2-CH3-VL(A2)-VH(A1)-VL(A1)-VH(A2), or VL(B)-VH(B)-hinge-CH2-CH3-VH(A2)-VL(A1)-VH(A1)-VL(A2)), and another polypeptide chain in the arrangement VH(B)-VL(B)-hinge-CH2-CH3-VL(A4)-VH(A3)-VL(A3)-VH(A4) (or less preferred VH(B)-VL(B)-hinge-CH2-CH3-VH(A4)-VL(A3)-VH(A3)-VL(A4), VL(B)-VH(B)-hinge-CH2-CH3-VL(A4)-VH(A3)-VL(A3)-VH(A4), or VL(B)-VH(B)-hinge-CH2-CH3-VH(A4)-VL(A3)-VH(A3)-VL(A4)). Illustrative examples for such antibody constructs are shown in SEQ ID NOs: 148-149.

A preferred antibody construct of the invention is preferably in a format as defined in the following. Such an antibody construct comprises an immunoglobulin that has two scDb fragments fused to the C-termini of the heavy chains, optionally via a linker, which is preferably a glycine serine linker or a serine linker, preferably a glycine serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 6), such as (GGGGS)₂ (SEQ ID NO: 7), (GGGGS)₄ (SEQ ID NO: 8), or preferably (GGGGS)₆ (SEQ ID NO: 9). Other illustrative examples for linkers are shown in SEQ ID NOs: 2-5. One of the two scDb comprises two first binding domains (A1 and A2), while the other scDb also comprises two first binding domains (A3 and A4). A second binding domains (B) and a third binding domain (C) are fused to the N termini of the Fc regions. Such an antibody construct may comprise two polypeptide chains, one polypeptide chain in the arrangement VH(B)-VL(B)-hinge-CH2-CH3-VL(A2)-VH(A1)-VL(A1)-VH(A2) (or less preferred VH(B)-VL(B)-hinge-CH2-CH3-VH(A2)-VL(A1)-VH(A1)-VL(A2), VL(B)-VH(B)-hinge-CH2-CH3-VL(A2)-VH(A1)-VL(A1)-VH(A2), or VL(B)-VH(B)-hinge-CH2-CH3-VH(A2)-VL(A1)-VH(A1)-VL(A2)), and another polypeptide chain in the arrangement VH(C)-VL(C)-hinge-CH2-CH3-VL(A4)-VH(A3)-VL(A3)-VH(A4) (or less preferred VH(C)-VL(C)-hinge-CH2-CH3-VH(A4)-VL(A3)-VH(A3)-VL(A4), VL(C)-VH(C)-hinge-CH2-CH3-VL(A4)-VH(A3)-VL(A3)-VH(A4), or VL(C)-VH(C)-hinge-CH2-CH3-VH(A4)-VL(A3)-VH(A3)-VL(A4)).

A preferred antibody construct of the invention is preferably in a format as essentially shown in FIG. 1C. Such an antibody construct comprises an immunoglobulin that has two scDb fragments fused to the C-termini of the heavy chains, optionally via a linker, which is preferably a glycine serine linker or a serine linker, preferably a glycine serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 6), such as (GGGGS)₂ (SEQ ID NO: 7), (GGGGS)₄ (SEQ ID NO: 8), or preferably (GGGGS)₆ (SEQ ID NO: 9). Other illustrative examples for linkers are shown in SEQ ID NOs: 2-5. One of the two scDb comprises two first binding domains (A1 and A2), while the other scDb also comprises two first binding domains (A3 and A4). Two second binding domains (B) are formed by the binding sites of the immunoglobulin. Such an antibody construct may comprise four polypeptide chains, two light chains in the arrangements VL(B)-CL and VL(B)-CL, one heavy chain fused to a scDb in the arrangement VH(B)-CH1-hinge-CH2-CH3-VL(A2)-VH(A1)-VL(A1)-VH(A2) (or less preferred or less preferred VH(B)-CH1-hinge-CH2-CH3-VH(A2)-VL(A1)-VH(A1)-VL(A2), VH(B)-CH1-hinge-CH2-CH3-VL(A2)-VH(A1)-VL(A1)-VH(A2), or VH(B)-CH1-hinge-CH2-CH3-VH(A2)-VL(A1)-VH(A1)-VL(A2)), and one heavy chain fused to an scDb in the arrangement VH(B)-CH1-hinge-CH2-CH3-VL(A4)-VH(A3)-VL(A3)-VH(A4) (or less preferred VH(B)-CH1-hinge-CH2-CH3-VH(A4)-VL(A3)-VH(A3)-VL(A4), VH(B)-CH1-hinge-CH2-CH3-VL(A4)-VH(A3)-VL(A3)-VH(A4), or VH(B)-CH1-hinge-CH2-CH3-VH(A4)-VL(A3)-VH(A3)-VL(A4)). Illustrative examples for such antibody constructs are shown in SEQ ID NOs: 162-163.

A preferred antibody construct of the invention is preferably in a format as defined in the following. Such an antibody construct comprises an immunoglobulin that has two scDb fragments fused to the C-termini of the heavy chains, optionally via a linker, which is preferably a glycine serine linker or a serine linker, preferably a glycine serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 6), such as (GGGGS), (SEQ ID NO: 7), (GGGGS); (SEQ ID NO: 8), or preferably (GGGGS)₆ (SEQ ID NO: 9). Other illustrative examples for linkers are shown in SEQ ID NOs: 2-5. One of the two scDb comprises two first binding domains (A1 and A2), while the other scDb also comprises two first binding domains (A3 and A4) One second binding domains (B) and one third binding domain (C) are formed by the binding sites of the immunoglobulin. Such an antibody construct may comprise four polypeptide chains, two light chains in the arrangements VL(B)-CL and VL(C)-CL, one heavy chain fused to a scDb in the arrangement VH(B)-CH1-hinge-CH2-CH3-VL(A2)-VH(A1)-VL(A1)-VH(A2) (or less preferred or less preferred VH(B)-CH1-hinge-CH2-CH3-VH(A2)-VL(A1)-VH(A1)-VL(A2), VH(B)-CH1-hinge-CH2-CH3-VL(A2)-VH(A1)-VL(A1)-VH(A2), or VH(B)-CH1-hinge-CH2-CH3-VH(A2)-VL(A1)-VH(A1)-VL(A2)), and one heavy chain fused to an scDb in the arrangement VH(C)-CH1-hinge-CH2-CH3-VL(A4)-VH(A3)-VL(A3)-VH(A4) (or less preferred VH(C)-CH1-hinge-CH2-CH3-VH(A4)-VL(A3)-VH(A3)-VL(A4), VH(C)-CH1-hinge-CH2-CH3-VL(A4)-VH(A3)-VL(A3)-VH(A4), or VH(C)-CH1-hinge-CH2-CH3-VH(A4)-VL(A3)-VH(A3)-VL(A4)).

In the antibody constructs of the disclosure, a first dimer of two first binding domains (A1A2) and a second dimer of two first binding domains (A3A4) can also be fused to the N-termini of a pair (e.g. a dimer) of two constant domains of an antibody, such as a pair of two CH3 domains, a pair of two CH2 domains, or a pair of a CH1 domain and a CL domain. In a preferred embodiment, a first dimer (A1A2) is fused to the N-terminus of a CH2 domain and a second dimer (A3A4) is fused to the N-terminus of another CH2 domain. In a preferred embodiment, a first dimer (A1A2) and a second dimer (A3A4) are fused to two N-termini of a Fc region. It is preferred for the antibody constructs of the disclosure that a first dimer (A1A2) is fused to the N-terminus of a first hinge domain and a second dimer (A3A4) is fused to the N-terminus of a second hinge domain. Such a fusion format is illustratively shown in FIG. 1D. The first dimer (A1A2) and/or second dimer (A3A4) may be fused to a constant domain of an antibody via a linker disclosed herein (such as a glycine serine linker or a serine linker, preferably a glycine serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 6), such as (GGGGS)₂ (SEQ ID NO: 7), (GGGGS)₄ (SEQ ID NO: 8), or preferably (GGGGS)₆ (SEQ ID NO: 9). Other illustrative examples for linkers are shown in SEQ ID NOs: 2-5) or a hinge domain, with a hinge domain being preferred.

Generally, a hinge domain comprised in an antibody construct of the disclosure may comprise a full-length hinge domain, such as a hinge domain shown in SEQ ID NO: 26. The hinge domain may also comprise a shortened and/or modified hinge domain. A shortened hinge domain may comprise the upper hinge domain as e.g. shown in SEQ ID NO: 27 or the middle hinge domain as e.g. shown in SEQ ID NO: 28, but not the entire hinge domain, with the latter being preferred. Preferred hinge domains in the context of the invention show modulated flexibility relative to an antibody construct having the wild type hinge domain as described in Dall'Acqua et al (J Immunol. 2006 Jul. 15; 177 (2): 1129-38) or in WO 2009/006520. A hinge domain showing reduced flexibility is preferred for some antibody constructs of the disclosure, in particular if the dimer (A1A2) and/or second dimer (A3A4) are fused to the hinge domain. Moreover, preferred hinge domains are characterized to consist of less than 25 aa residues. More preferably, the length of the hinge is 10 to 20 aa residues. A hinge domain comprised in an antibody construct of the disclosure may also comprise or consists of the IgG2 subtype hinge sequence ERKCCVECPPCP (SEQ ID NO: 23), the IgG3 subtype hinge sequence ELKTPLDTTHTCPRCP (SEQ ID NO: 30) or ELKTPLGDTTHTCPRCP (SEQ ID NO: 131), and/or the IgG4 subtype hinge sequence ESKYGPPCPSCP (SEQ ID NO: 132). Further hinge domains that can be used in the context of the present invention are known to the skilled person and are e.g. described in WO 2017/134140.

When a first dimer (A1A2) is fused to the N-terminus of a CH2 domain and a second dimer (A3A4) is fused to the N-terminus of another CH2 domain, such as when a first dimer (A1A2) and a second dimer (A3A4) are fused to two N-termini of a Fc region, a second binding domain (B) can be fused to the C terminus of a Fc region, optionally via a linker disclosed herein. The second binding domain is preferably in form of an scFv. The scFv is preferably fused to the Fc region via its VH domain. Such an antibody construct may comprise two polypeptide chains, one polypeptide chain in the arrangement VL(A2)-VH(A1)-VL(A1)-VH(A2)-hinge-CH2-CH3-VH(B)-VL(B) (or less preferred VH(A2)-VL(A1)-VH(A1)-VL(A2)-hinge-CH2-CH3-VH(B)-VL(B), VL(A2)-VH(A1)-VL(A1)-VH(A2)-hinge-CH2-CH3-VL(B)-VH(B), or VH(A2)-VL(A1)-VH(A1)-VL(A2)-hinge-CH2-CH3-VL(B)-VH(B)), and another polypeptide chain in the arrangement VL(A4)-VH(A3)-VL(A3)-VH(A4)-hinge-CH2-CH3 (or less preferred VH(A4)-VL(A3)-VH(A3)-VL(A4)-hinge-CH2-CH3). Illustrative examples for such antibody constructs are shown in SEQ ID NOs: 150-151 and 156-157.

When a first dimer (A1A2) is fused to the N-terminus of a CH2 domain and a second dimer (A3A4) is fused to the N-terminus of another CH2 domain, such as when a first dimer (A1A2) and a second dimer (A3A4) are fused to two N-termini of a Fc region, two second binding domains (B) can be fused to the C termini of a Fc region, optionally via a linker disclosed herein. The second binding domains are preferably in form of an scFv. An scFv is preferably fused to the Fc region via its VH domain. Such an antibody construct may comprise two polypeptide chains, one polypeptide chain in the arrangement VL(A2)-VH(A1)-VL(A1)-VH(A2)-hinge-CH2-CH3-VH(B)-VL(B) (or less preferred VH(A2)-VL(A1)-VH(A1)-VL(A2)-hinge-CH2-CH3-VH(B)-VL(B), VL(A2)-VH(A1)-VL(A1)-VH(A2)-hinge-CH2-CH3-VL(B)-VH(B), or VH(A2)-VL(A1)-VH(A1)-VL(A2)-hinge-CH2-CH3-VL(B)-VH(B)), and another polypeptide chain in the arrangement VL(A4)-VH(A3)-VL(A3)-VH(A4)-hinge-CH2-CH3-VH(B)-VL(B) (or less preferred VH(A4)-VL(A3)-VH(A3)-VL(A4)-hinge-CH2-CH3-VH(B)-VL(B), VL(A4)-VH(A3)-VL(A3)-VH(A4)-hinge-CH2-CH3-VL(B)-VH(B), or VH(A4)-VL(A3)-VH(A3)-VL(A4)-hinge-CH2-CH3-VL(B)-VH(B)).

When a first dimer (A1A2) is fused to the N-terminus of a CH2 domain and a second dimer (A3A4) is fused to the N-terminus of another CH2 domain, such as when a first dimer (A1A2) and a second dimer (A3A4) are fused to two N-termini of a Fc region, a second binding domains (B) and a third binding domain (C) can be fused to the C termini of a Fc region, optionally via a linker disclosed herein. The second binding and/or third binding domain are preferably in form of an scFv. An scFv is preferably fused to the Fc region via its VH domain. Such an antibody construct may comprise two polypeptide chains, one polypeptide chain in the arrangement VL(A2)-VH(A1)-VL(A1)-VH(A2)-hinge-CH2-CH3-VH(B)-VL(B) (or less preferred VH(A2)-VL(A1)-VH(A1)-VL(A2)-hinge-CH2-CH3-VH(B)-VL(B), VL(A2)-VH(A1)-VL(A1)-VH(A2)-hinge-CH2-CH3-VL(B)-VH(B), or VH(A2)-VL(A1)-VH(A1)-VL(A2)-hinge-CH2-CH3-VL(B)-VH(B)), and another polypeptide chain in the arrangement VL(A4)-VH(A3)-VL(A3)-VH(A4)-hinge-CH2-CH3-VH(C)-VL(C) (or less preferred VH(A4)-VL(A3)-VH(A3)-VL(A4)-hinge-CH2-CH3-VH(C)-VL(C), VL(A4)-VH(A3)-VL(A3)-VH(A4)-hinge-CH2-CH3-VL(C)-VH(C), or VH(A4)-VL(A3)-VH(A3)-VL(A4)-hinge-CH2-CH3-VL(C)-VH(C)).

In an antibody construct of the disclosure, a first dimer of two first binding domains (A1A2) may be fused to the N terminus of a first hinge-CH2-CH3 element of a fourth domain (D), while a second dimer of two first binding domains (A3A4) may be fused to the C terminus of a second hinge-CH2-CH3 element of the fourth domain (D). In such a case, a second binding domain (B) may be fused to the C terminus of the first hinge-CH2-CH3 element of the fourth domain (D). The first and second dimers (A1A2) and (A3A4) are preferably in form of a scDb. The second binding domain (B) is preferably in form of an scFv. Such a fusion format is illustratively shown in FIG. 1E. Such an antibody construct may comprise two polypeptide chains, one polypeptide chain in the arrangement VL(A2)-VH(A1)-VL(A1)-VH(A2)-hinge-CH2-CH3-VH(B)-VL(B) (or less preferred VH(A2)-VL(A1)-VH(A1)-VL(A2)-hinge-CH2-CH3-VH(B)-VL(B), VL(A2)-VH(A1)-VL(A1)-VH(A2)-hinge-CH2-CH3-VL(B)-VH(B), or VH(A2)-VL(A1)-VH(A1)-VL(A2)-hinge-CH2-CH3-VL(B)-VH(B)), and another polypeptide chain in the arrangement hinge-CH2-CH3-VL(A4)-VH(A3)-VL(A3)-VH(A4) (or less preferred hinge-CH2-CH3-VH(A4)-VL(A3)-VH(A3)-VL(A4)). Illustrative examples for such antibody constructs are shown in SEQ ID NOs: 154-155 and 160-161.

In an antibody construct of the disclosure, a first dimer of two first binding domains (A1A2) may be fused to the N terminus of a first hinge-CH2-CH3 element of a fourth domain (D), while a second dimer of two first binding domains (A3A4) may be fused to the C terminus of a second hinge-CH2-CH3 element of the fourth domain (D). In such a case, a second binding domain (B) may be fused to the N terminus of the second hinge-CH2-CH3 element of the fourth domain (D). The first and second dimers (A1A2) and (A3A4) are preferably in form of a scDb. The second binding domain(s) (B) is preferably in form of an scFv. Such an antibody construct may comprise two polypeptide chains, one polypeptide chain in the arrangement VL(A2)-VH(A1)-VL(A1)-VH(A2)-hinge-CH2-CH3 (or less preferred VH(A2)-VL(A1)-VH(A1)-VL(A2)-hinge-CH2-CH3), and another polypeptide chain in the arrangement VH(B)-VL(B)-hinge-CH2-CH3-VL(A4)-VH(A3)-VL(A3)-VH(A4) (or less preferred VH(B)-VL(B)-hinge-CH2-CH3-VH(A4)-VL(A3)-VH(A3)-VL(A4), VL(B)-VH(B)-hinge-CH2-CH3-VL(A4)-VH(A3)-VL(A3)-VH(A4), or VL(B)-VH(B)-hinge-CH2-CH3-VH(A4)-VL(A3)-VH(A3)-VL(A4)).

In an antibody construct of the disclosure, a first dimer of two first binding domains (A1A2) may be fused to the N terminus of a first hinge-CH2-CH3 element of a fourth domain (D), while a second dimer of two first binding domains (A3A4) may be fused to the C terminus of a second hinge-CH2-CH3 element of the fourth domain (D). In such a case, one second binding domain (B) may be fused to the C terminus of the first hinge-CH2-CH3 element of the fourth domain (D) and another second binding domain (B) may be fused to the N terminus of the second hinge-CH2-CH3 element of the fourth domain (D). The first and second dimers (A1A2) and (A3A4) are preferably in form of a scDb. The second binding domains (B) are preferably in form of an scFv. Such an antibody construct may comprise two polypeptide chains, one polypeptide chain in the arrangement VL(A2)-VH(A1)-VL(A1)-VH(A2)-hinge-CH2-CH3-VH(B)-VL(B) (or less preferred VH(A2)-VL(A1)-VH(A1)-VL(A2)-hinge-CH2-CH3-VH(B)-VL(B), VL(A2)-VH(A1)-VL(A1)-VH(A2)-hinge-CH2-CH3-VL(B)-VH(B), or VH(A2)-VL(A1)-VH(A1)-VL(A2)-hinge-CH2-CH3-VL(B)-VH(B)), and another polypeptide chain in the arrangement VH(B)-VL(B)-hinge-CH2-CH3-VL(A4)-VH(A3)-VL(A3)-VH(A4) (or less preferred VH(B)-VL(B)-hinge-CH2-CH3-VH(A4)-VL(A3)-VH(A3)-VL(A4), VL(B)-VH(B)-hinge-CH2-CH3-VL(A4)-VH(A3)-VL(A3)-VH(A4), or VL(B)-VH(B)-hinge-CH2-CH3-VH(A4)-VL(A3)-VH(A3)-VL(A4)).

In an antibody construct of the disclosure, a first dimer of two first binding domains (A1A2) may be fused to the N terminus of a first hinge-CH2-CH3 element of a fourth domain (D), while a second dimer of two first binding domains (A3A4) may be fused to the C terminus of a second hinge-CH2-CH3 element of the fourth domain (D). In such a case, a second binding domain (B) may be fused to the C terminus of the first hinge-CH2-CH3 element of the fourth domain (D) and a third binding domain (C) may be fused to the N terminus of the second hinge-CH2-CH3 element of the fourth domain (D). The first and second dimers (A1A2) and (A3A4) are preferably in form of a scDb. The second binding domain (B) and the third binding domain (C) are preferably in form of an scFv. Such an antibody construct may comprise two polypeptide chains, one polypeptide chain in the arrangement VL(A2)-VH(A1)-VL(A1)-VH(A2)-hinge-CH2-CH3-VH(B)-VL(B) (or less preferred VH(A2)-VL(A1)-VH(A1)-VL(A2)-hinge-CH2-CH3-VH(B)-VL(B), VL(A2)-VH(A1)-VL(A1)-VH(A2)-hinge-CH2-CH3-VL(B)-VH(B), or VH(A2)-VL(A1)-VH(A1)-VL(A2)-hinge-CH2-CH3-VL(B)-VH(B)), and another polypeptide chain in the arrangement VH(C)-VL(C)-hinge-CH2-CH3-VL(A4)-VH(A3)-VL(A3)-VH(A4) (or less preferred VH(C)-VL(C)-hinge-CH2-CH3-VH(A4)-VL(A3)-VH(A3)-VL(A4), VL(C)-VH(C)-hinge-CH2-CH3-VL(A4)-VH(A3)-VL(A3)-VH(A4), or VL(C)-VH(C)-hinge-CH2-CH3-VH(A4)-VL(A3)-VH(A3)-VL(A4)).

In an antibody construct of the disclosure, a first dimer of two first binding domains (A1A2) may be fused to the N terminus of a first hinge-CH2-CH3 element of a fourth domain (D), while a second dimer of two first binding domains (A3A4) may be fused to the C terminus of a second hinge-CH2-CH3 element of the fourth domain (D). In such a case, a third binding domain (C) may be fused to the C terminus of the first hinge-CH2-CH3 element of the fourth domain (D) and a second binding domain (B) may be fused to the N terminus of the second hinge-CH2-CH3 element of the fourth domain (D). The first and second dimers (A1A2) and (A3A4) are preferably in form of a scDb. The second binding domain (B) and the third binding domain (C) are preferably in form of an scFv. Such an antibody construct may comprise two polypeptide chains, one polypeptide chain in the arrangement VL(A2)-VH(A1)-VL(A1)-VH(A2)-hinge-CH2-CH3-VH(C)-VL(C) (or less preferred VH(A2)-VL(A1)-VH(A1)-VL(A2)-hinge-CH2-CH3-VH(C)-VL(C), VL(A2)-VH(A1)-VL(A1)-VH(A2)-hinge-CH2-CH3-VL(C)-VH(C), or VH(A2)-VL(A1)-VH(A1)-VL(A2)-hinge-CH2-CH3-VL(C)-VH(C)), and another polypeptide chain in the arrangement VH(B)-VL(B)-hinge-CH2-CH3-VL(A4)-VH(A3)-VL(A3)-VH(A4) (or less preferred VH(B)-VL(B)-hinge-CH2-CH3-VH(A4)-VL(A3)-VH(A3)-VL(A4), VL(B)-VH(B)-hinge-CH2-CH3-VL(A4)-VH(A3)-VL(A3)-VH(A4), or VL(B)-VH(B)-hinge-CH2-CH3-VH(A4)-VL(A3)-VH(A3)-VL(A4)).

Generally, antibody constructs described herein having two scDb fragments comprising the four first binding domains (A1-A4) that are fused to the C-termini of the two heavy chains are preferred. Out of these antibody constructs, those that comprise one or two second binding domains (B) in form of an scFv that are fused to one or two N termini of the Fc region are most preferred. Less preferred are antibody constructs described herein which comprise one scDb fragment comprising two first binding sites (A1 and A2 or A3 and A4) that is fused to the C terminus of a heavy chain and that comprise another scDb fragment comprising two first binding sites (A3 and A4 or A1 and A2) that is fused to an N terminus of the Fc region. Not preferred are antibody constructs described herein that have two scDb fragments comprising the four first binding domains (A1-A4) that are fused to the two N termini of the Fc region.

Ideally, the distance between the binding site of the first binding domains (A1, A2, A3, A4) are short. It is thus preferred that the two binding domains are within the distance of about 30 or less, preferably about 25 nm or less, more preferably about 22 nm or less, more preferably about 20 nm or less, more preferably about 19 nm or less, more preferably about 18 nm or less, more preferably about 17 nm or less, more preferably about 16 nm or less, more preferably about 15 nm or less, more preferably about 14 nm or less, more preferably about 13 nm or less, more preferably about 12 nm or less, more preferably about 11 nm or less, more preferably about 10 nm or less, more preferably about 9 nm or less, more preferably about 8 nm or less, more preferably about 7 nm or less, more preferably about 6 nm or less, more preferably about 5 nm or less. The distance is preferably determined from the center of the binding site. The distance between the domains are preferably measured between the two first binding domain (A1, A2, A3, A4, . . . ) that have the largest distance to each other. For determining the distance between two binding domains, crystal structures are preferred. Where crystal structures are not available, structural considerations according Rossmalen et al Biochemistry 2017, 56, 6565-6574, are preferably applied, in particular with regard to linkers.

Where an antibody construct of the invention comprises CH3 regions, modifications to the CH3 region can be introduced to improve heterodimeric pairing of the polypeptides comprising the CH3 regions. The CH3 regions can be altered by the “knob-into-holes” technology which is described in detail with several examples in e.g. WO 96/027011, Ridgway, J., B, et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M., et al., Nature Biotech 16 (1998) 677-681; Atwell, S., et al., J. Mol. Biol. 270 (1997) 26-35) and increases the yield.

Thus the antibody constructs of the disclosure may be further characterized in that the CH3 domain of one polypeptide chain and the CH3 domain of another polypeptide chain each meet at an interface which comprises an original interface between the antibody CH3 domains; wherein the interface is altered to promote the formation of the antibody construct. An alteration may be characterized in that: a) the CH3 domain of one polypeptide chain is altered, so that within the original interface the CH3 domain of one polypeptide chain that meets the original interface of the CH3 domain of the other polypeptide chain within the antibody construct, an amino acid residue is replaced with an amino acid residue having a larger side chain volume, thereby generating a protuberance within the interface of the CH3 domain of one polypeptide chain which is positionable in a cavity within the interface of the CH3 domain of the other polypeptide chain and b) the CH3 domain of the other polypeptide chain is altered, so that within the original interface of the second CH3 domain that meets the original interface of the first CH3 domain within the antibody construct an amino acid residue is replaced with an amino acid residue having a smaller side chain volume, thereby generating a cavity within the interface of the second CH3 domain within which a protuberance within the interface of the first CH3 domain is positionable.

Preferably the amino acid residue having a larger side chain volume is selected from the group consisting of arginine (R), phenylalanine (F), tyrosine (Y), tryptophan (W). Preferably the amino acid residue having a smaller side chain volume is selected from the group consisting of alanine (A), serine(S), threonine (T), valine (V).

Both CH3 domains further be altered by the introduction of cysteine (C) as amino acid in the corresponding positions of each CH3 domain such that a disulfide bridge between both CH3 domains can be formed.

In a preferred embodiment, the antibody construct comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M, et al., Nature Biotech 16 (1998) 677-681) e.g. by introducing a Y349C mutation into the CH3 domain of the “knobs chain” and a E356C mutation or a S354C mutation into the CH3 domain of the “hole chain”. Alternatively, the antibody construct may comprise a T366Y in the CH3 domain of the “knobs chain” and a Y407T mutation in the “hole chain”. Other knobs-in-holes technologies that can also be used are described in Labrijn A F, Janmaat M L, Reichert J M, Parren P. Bispecific antibodies: a mechanistic review of the pipeline. Nat Rev Drug Discov 2019; 18:585-608. Preferred versions of knob chain CH2-CH3 heavy chain constant domains are shown in SEQ ID NOs: 44, 46, 48, 50, 52, 54, 56, and 58. Preferred versions of hole chain CH2-CH3 heavy chain constant domains are shown in SEQ ID NOs: 43, 45, 47, 49, 51, 53, 55, and 57.

In a preferred antibody construct, the at least four first binding domains (A) are capable of specifically binding CD16A, which preferably includes the capacity to discriminate between CD16A and CD16B. With other words, the at least four first binding domains (A) preferably binds CD16A with higher affinity than CD16B, which may be at least about 10-fold higher, at least about 100-fold higher, or at least about 1000-fold higher. More preferably, the at least four first binding domains (A) do not essentially bind CD16B. It is thus understood that the first binding domain is preferably not a non-silenced CH2 domain, i.e. a CH2 domain that is capable of binding both CD16A and CD16B.

Accordingly the at least four first binding domains (A) preferably binds to an epitope of CD16A which comprises amino acid residues of the C-terminal sequence SFFPPGYQ (positions 201-209 of SEQ ID NO: 13), and/or residue G147 and/or residue Y158 of CD16A, which are not present in CD16B. It is preferred in the context of the invention that the first binding domain, which binds CD16A on the surface of an effector cell binds to an epitope on CD16A, which is membrane proximal relative to the physiological Fcγ receptor binding domain of CD16A. A binding domain that specifically binds to an epitope comprising Y158 is preferred, because this epitope is proximal to the cell membrane and thus further contributes to reducing the likelihood of simultaneously binding a second immune effector cell. Examples for respective binding domains are characterized e.g. by the following groups of CDRs:
CDR-H1 as depicted in SEQ ID NO: 77, a CDR-H2 as depicted in SEQ ID NO: 78, a CDR-H3 as depicted in SEQ ID NO: 79, a CDR-L1 as depicted in SEQ ID NO: 80, a CDR-L2 as depicted in SEQ ID NO: 81, a CDR-13 as depicted in SEQ ID NO: 82 and binding domains which bind to the same epitope;
CDR-H1 as depicted in SEQ ID NO: 83, a CDR-H2 as depicted in SEQ ID NO: 84, a CDR-H3 as depicted in SEQ ID NO: 85, a CDR-L1 as depicted in SEQ ID NO: 86, a CDR-1.2 as depicted in SEQ ID NO: 87, a CDR-L3 as depicted in SEQ ID NO: 88 and binding domains which bind to the same epitope; and
CDR-H1 as depicted in SEQ ID NO: 77, a CDR-H2 as depicted in SEQ ID NO: 89, a CDR-H3 as depicted in SEQ ID NO: 79, a CDR-L1 as depicted in SEQ ID NO: 80, a CDR-L2 as depicted in SEQ ID NO: 81, a CDR-L3 as depicted in SEQ ID NO: 82 and binding domains which bind to the same epitope.

Preferred CD16A binding domains are characterized by the following groups of CDRs: CDR-H1 as depicted in SEQ ID NO: 83, a CDR-H2 as depicted in SEQ ID NO: 84, a CDR-H3 as depicted in SEQ ID NO: 85, a CDR-L1 as depicted in SEQ ID NO: 86, a CDR-L2 as depicted in SEQ ID NO: 87, a CDR-L3 as depicted in SEQ ID NO: 88 and binding domains which bind to the same epitope.

Examples for such CD16A binder are also described in WO2020043670.

In some embodiments, the at least four first binding domains (A) comprise the same CDR sequences. In some embodiments, one or more, preferably all, of the at least four first binding domains (A) comprise a VH region comprising CDR-H1, CDR-H2 and CDR-H3 and a VL region comprising CDR-L1, CDR-L2 and CDR-L3 selected from: CDR-H1 as depicted in SEQ ID NO: 77, a CDR-H2 as depicted in SEQ ID NO: 78, a CDR-H3 as depicted in SEQ ID NO: 79, a CDR-L1 as depicted in SEQ ID NO: 80, a CDR-L2 as depicted in SEQ ID NO: 81, a CDR-L3 as depicted in SEQ ID NO: 82 and binding domains which bind to the same epitope;
CDR-H1 as depicted in SEQ ID NO: 83, a CDR-H2 as depicted in SEQ ID NO: 84, a CDR-H3 as depicted in SEQ ID NO: 85, a CDR-L1 as depicted in SEQ ID NO: 86, a CDR-L2 as depicted in SEQ ID NO: 87, a CDR-L3 as depicted in SEQ ID NO: 88 and binding domains which bind to the same epitope, which is preferred; and
CDR-H1 as depicted in SEQ ID NO: 77, a CDR-H2 as depicted in SEQ ID NO: 89, a CDR-H3 as depicted in SEQ ID NO: 79, a CDR-L1 as depicted in SEQ ID NO: 80, a CDR-L2 as depicted in SEQ ID NO: 81, a CDR-L3 as depicted in SEQ ID NO: 82 and binding domains which bind to the same epitope.

In some embodiments, the at least four first binding domains (A) comprise the same VL and VH sequences. In some preferred embodiments, one or more, preferably all, of the at least four first binding domains (A) comprises a pair of VH- and VL-chains having a sequence as depicted in the pairs of sequences selected form the group consisting of SEQ ID NOs: 59 and 68; SEQ ID NOs: 60 and 69, and SEQ ID NOs: 61 and 70, with SEQ ID NO 60 and 69 being preferred. In some embodiments, the at least four first binding domains (A) comprise the same amino acid sequences

In some embodiments, one or more, preferably all, of the at least four first binding domains (A) comprises a VH domain comprising the following three heavy chain CDRs and a VL domain comprising the following three light chain CDRs: a CDR-H1 as depicted in SEQ ID NO: 83, a CDR-H2 as depicted in SEQ ID NO: 84, a CDR-H3 as depicted in SEQ ID NO: 85, a CDR-L1 as depicted in SEQ ID NO: 86, a CDR-L2 as depicted in SEQ ID NO: 87, a CDR-L3 as depicted in SEQ ID NO: 88.

In some embodiments, one or more, preferably all, of the at least four first binding domain (A) comprises a pair of VH- and VL-chains having a sequence as depicted in the pairs of sequences selected form the group consisting of SEQ ID NOs: 60 and 69.

Antibodies against first targets (A′) of the disclosure are well known in the art. Antibodies against CD16A are e.g. described in WO2020043670. Antibodies against CD56 are e.g. described in WO2012138537 and WO2017023780. Antibodies against NKG2A are e.g. described in WO2008009545, WO2009092805, WO2016032334, WO2020094071, WO2020102501. Antibodies against NKG2D are e.g. described in WO2009077483, WO2018148447, WO2019157366. Antibodies against NKp30 are e.g. described in WO2020172605. Antibodies against NKp46 are e.g. described in WO2011086179 and WO2016209021. Antibodies against DNAM-1 are e.g. described in WO2013140787. Antibodies against SLAMF7 are e.g. described in US2018208653. Antibodies against OX40 are e.g. described in WO2007062245, US2010136030, US2019100596, WO2013008171, WO2013028231. Antibodies against CD47, SIRPα are e.g. described in WO9727873, WO2005044857, US2014161799. Antibodies against CD89 are e.g. described in WO02064634, WO2020084056. Antibodies against CD96 are e.g. described in WO2019091449. Antibodies against CD137 are e.g. described in WO2005035584. WO2006088464, US2006188439. Antibodies against CD160 are e.g. described in US2012003224, US2013122006. Antibodies against TIGIT are e.g. described in US2020040082 and WO2019062832. Antibodies against nectin-4 are e.g. described in WO2018158398. Antibodies against PD-1 are e.g. described in WO2009014708, US2012237522, US2013095098, and US2011229461. Antibodies against PD-L1 are e.g. described in US2012237522, WO2014022758, WO2014055897, and WO2014195852. Antibodies against LAG-3 are e.g. described in WO2008132601, US2016176965, and WO2010019570. Antibodies against CTLA-4 are e.g. described in WO2005092380, US2009252741, and WO2006066568. Antibodies against TIM-3 are e.g. described in US2014134639, WO2011155607, and WO2015117002. Antibodies against KIR2DS1-5 are e.g. described in WO2016031936. Antibodies against CD3 are e.g. described in U.S. Pat. No. 6,750,325, WO9304187, and WO9516037.

In some preferred embodiments, the at least four first binding domains (A) are specific for NKG2D. One or more, preferably all, of the at least four first binding domains (A) preferably comprise the following three heavy chain CDRs and three light chain CDRs: a CDR-H1 as depicted in SEQ ID NO: 96, a CDR-H2 as depicted in SEQ ID NO: 97, a CDR-H3 as depicted in SEQ ID NO: 98, a CDR-L1 as depicted in SEQ ID NO: 99, a CDR-L2 as depicted in SEQ ID NO: 100, a CDR-L3 as depicted in SEQ ID NO: 101.

In some preferred embodiments, one or more, preferably all, of the at least four first binding domains (A) comprises a pair of VH- and VL-chains having a sequence as depicted in the pairs of sequences of SEQ ID NOs: 63 and 72.

In some preferred embodiments, the at least four first binding domains (A) are specific for NKp46. One or more, preferably all, of the at least four first binding domains (A) preferably comprise the following three heavy chain CDRs and three light chain CDRs: a CDR-H1 as depicted in SEQ ID NO: 90, a CDR-H2 as depicted in SEQ ID NO: 91, a CDR-H3 as depicted in SEQ ID NO: 92, a CDR-L1 as depicted in SEQ ID NO: 93, a CDR-L2 as depicted in SEQ ID NO: 94, a CDR-L3 as depicted in SEQ ID NO: 95.

In some preferred embodiments, one or more, preferably all, of the at least four first binding domains (A) comprises a pair of VH- and VL-chains having a sequence as depicted in the pairs of sequences of SEQ ID NOs: 62 and 71.

In some embodiments, the second binding domain (B) is specific for a second target (B′) that is a tumor associated antigen. The second target (B′) is preferably selected from the group consisting of CD19, CD20, CD22, CD30, CD33, CD52, CD70, CD74, CD79b, CD123, CLL1, BCMA, FCRH5, EGFR, EGFRvlll, HER2, GD2.

In some embodiments, the third binding domain (B) is specific for a third target (B′) that is a tumor associated antigen. The third target (B′) is preferably selected from the group consisting of CD19, CD20, CD22, CD30, CD33, CD52, CD70, CD74, CD79b, CD123, CLL1, BCMA, FCRH5, EGFR, EGFRvlll, HER2, GD2.

These cell surface antigens on the surface of target cells are connected with specific disease entities. CD30 is a cell surface antigen characteristic for malignant cells in Hodgkin lymphoma. CD19, CD20, CD22, CD70, CD74 and CD79b are cell surface antigens characteristic for malignant cells in Non-Hodgkin lymphomas (Diffuse large B-cell lymphoma (DLBCL), Mantle cell lymphoma (MCL), Follicular lymphoma (FL), T-cell lymphomas (both peripheral and cutaneous, including transformed mycosis fungoides/Sezary syndrome TMF/SS and Anaplastic large-cell lymphoma (ALCL)). CD52, CD33, CD123, CLL1 are cell surface antigens characteristic for malignant cells in Leukemias (Chronic lymphocytic leukemia (CLL), Acute lymphoblastic leukemia (ALL), Acute myeloid leukemia (AML)). BCMA, FCRH5 are cell surface antigens characteristic for malignant cells in Multiple Myeloma. EGFR, HER2, GD2 are cell surface antigens characteristic for solid cancers (Triple-negative breast cancer (TNBC), breast cancer BC, Colorectal cancer (CRC), Non-small-cell lung carcinoma (NSCLC), Small-cell carcinoma (SCLC also known as “small-cell lung cancer”, or “oat-cell carcinoma”), Prostate cancer (PC), Glioblastoma (also known as glioblastoma multiforme (GBM)).

Antibodies against such targets are well known in the art. Antibodies against CD19 are e.g. described in WO2018002031, WO2015157286, and WO2016112855. Antibodies against CD20 are e.g. described in WO2017185949, US2009197330, and WO2019164821. Antibodies against CD22 are e.g. described in WO2020014482, WO2013163519, U.S. Pat. No. 10,590,197. Antibodies against CD30 are e.g. described in WO2007044616, WO2014164067, and WO2020135426. Antibodies against CD33 are e.g. described in WO2019006280, WO2018200562, and WO2016201389. Antibodies against CD52 are e.g. described in WO2005042581, WO2011109662, and US2003124127. Antibodies against CD70 are e.g. described in US2012294863, WO2014158821, and WO2006113909. Antibodies against CD74 are e.g. described in WO03074567, US2014030273, and WO2017132617. Antibodies against CD79b are e.g. described in US2009028856, US2010215669, and WO2020088587. Antibodies against CD123 are e.g. described in US2017183413, WO2016116626, and U.S. Pat. No. 10,100,118. Antibodies against CLL1 are e.g. described in WO2020083406. Antibodies against BCMA are e.g. described in WO02066516, U.S. Pat. No. 10,745,486, and US2019112382. Antibodies against FCRH5 are e.g. described in US2013089497. Antibodies against EGFR are e.g. described in WO9520045, WO9525167, and WO02066058. Antibodies against EGFRvlll are e.g. described in WO2017125831. Antibodies against HER2 are e.g. described in US2011189168, WO0105425, and US2002076695. Antibodies against GD2 are e.g. described in WO8600909, WO8802006, and U.S. Pat. No. 5,977,316.

In some preferred embodiments, the second binding domain (B) and/or third binding domain (C) is specific for EGFR and preferably comprises a VH domain comprising the following three heavy chain CDRs and a VL domain comprising the following three light chain CDRs: a CDR-H1 as depicted in SEQ ID NO: 114, a CDR-H2 as depicted in SEQ ID NO: 115, a CDR-H3 as depicted in SEQ ID NO 116, a CDR-L1 as depicted in SEQ ID NO: 117, a CDR-L2 as depicted in SEQ ID NO: 118, a CDR-L3 as depicted in SEQ ID NO: 119.

In some preferred embodiments, the second binding domain (B) and/or third binding domain (C) comprises a pair of VH- and VL-chains having a sequence as depicted in the pairs of sequences of SEQ ID NOs: 66 and 75.

In some preferred embodiments, the second binding domain (B) and/or third binding domain (C) is specific for BCMA and preferably comprises a VH domain comprising the following three heavy chain CDRs and a VH domain comprising the following three light chain CDRs: a CDR-H1 as depicted in SEQ ID NO: 102, a CDR-H2 as depicted in SEQ ID NO: 103, a CDR-H3 as depicted in SEQ ID NO: 104, a CDR-L1 as depicted in SEQ ID NO: 105, a CDR-L2 as depicted in SEQ ID NO: 106, a CDR-L3 as depicted in SEQ ID NO: 107.

In some preferred embodiments, the second binding domain (B) and/or third binding domain (C) comprises a pair of VH- and VL-chains having a sequence as depicted in SEQ ID NOs: 64 and 73.

In some preferred embodiments, the second binding domain (B) and/or third binding domain (C) is specific for CD19 and preferably comprises a VH domain comprising the following three heavy chain CDRs and a VH domain comprising the following three light chain CDRs: a CDR-H1 as depicted in SEQ ID NO: 108, a CDR-H2 as depicted in SEQ ID NO: 109, a CDR-H3 as depicted in SEQ ID NO: 110, a CDR-L1 as depicted in SEQ ID NO: 111, a CDR-L2 as depicted in SEQ ID NO: 112, a CDR-L3 as depicted in SEQ ID NO: 113.

In some preferred embodiments, the second binding domain (B) and/or third binding domain (C) comprises a pair of VH- and VL-chains having a sequence as depicted in SEQ ID NOs: 65 and 74.

In some preferred embodiments, the second binding domain (B) and/or third binding domain (C) is specific for HER2 and preferably comprises a VH domain comprising the following three heavy chain CDRs and a VH domain comprising the following three light chain CDRs: a CDR-H1 as depicted in SEQ ID NO: 120, a CDR-H2 as depicted in SEQ ID NO: 121, a CDR-H3 as depicted in SEQ ID NO: 122, a CDR-L1 as depicted in SEQ ID NO: 123, a CDR-1.2 as depicted in SEQ ID NO: 124, a CDR-L3 as depicted in SEQ ID NO: 125.

In some preferred embodiments, the second binding domain (B) and/or third binding domain (C) comprises a pair of VH- and VL-chains having a sequence as depicted in SEQ ID NOs: 67 and 76.

An antibody construct of the invention is preferably an antibody construct selected from the group consisting of SEQ ID NOs: 148, 149, 150 and 151, 152 and 153, 154 and 155, 156 and 157, 158 and 159, 160 and 161, 162 and 163, 180-183, 190, and 191 and 192.

An antibody construct of the invention is preferably a variant of an antibody construct selected from the group consisting of SEQ ID NOs: 148, 149, 150 and 151, 152 and 153, 154 and 155, 156 and 157, 158 and 159, 160 and 161, 162 and 163, 180-183, 190, and 191 and 192, wherein the variant has at least 90%, preferably at least 95%, more preferably at least 98%, even more preferably at least 99% sequence identity to any one of these aforementioned antibody constructs, preferably provided that the CDR sequences comprised in these antibody constructs are not altered.

The antibody construct of the invention is characterized by inducing a low degree of fratricide, which is also referred to as a “reduced” degree of fratricide. The degree of fratricide can be measured in a cytotoxicity assay, such as an assay as essentially described in Example 7. Such an assay is preferably conducted as follows. For the calcein-release NK cell fratricide assays, enriched primary human NK cells are labeled with 10 μM of the fluorescent dye calcein AM for 30 min, and aliquots of 5×10⁴ labeled cells are seeded in individual wells of a round-bottom 96-well micro plate together with unlabeled, enriched autologous NK cells at an effector:target (E:T) ratio of 1:1 in the presence of 10 serial 1:5 dilutions of the indicated test or control antibody construct starting at 100 μg/mL, preferably in duplicates. Anti-CD38 IgG1 with daratumumab-derived Fab domains (IgAb_51, SEQ ID NOs: 166 and 167) are preferably used as a positive control. Control samples to measure spontaneous release, maximal release and antibody-independent lysis by effector cells are preferably tested in 4 replicates. After incubation for 4 h, 100 μL cell-free culture supernatant is harvested from each well to quantify the fluorescent calcein released from lysed target cells with a multiplate fluorescence reader. After subtracting the fluorescence of spontaneously lysed cells from all samples, the fluorescence of each sample should be normalized to the fluorescence of fully lysed cells to determine the specific lysis for a respective sample. Mean values of specific target cell lysis (%) and standard deviations (SD) can be plotted and in vitro potency (EC₅₀) and efficacy (Eₘₐₓ) can be determined by fitting the non-linear regression model to sigmoidal dose-response curves (variable slope) using GraphPad Prism (v6 and v7: GraphPad Software, La Jolla California USA).

In some embodiments, a “low degree of fratricide” means that the degree of fratricide of a test molecule, such as an antibody construct of the invention, is about 40% or lower. The degree of fratricide of an antibody construct of the invention is preferably about 35% or lower, more preferably about 30% or lower, more preferably about 25% or lower, more preferably about 22% or lower, more preferably about 20% or lower, more preferably about 19% or lower, more preferably about 18% or lower, more preferably about 17% or lower, more preferably about 16% or lower, more preferably about 15% or lower, more preferably about 14% or lower more preferably about 13% or lower, more preferably about 12% or lower, more preferably about 11% or lower, more preferably about 10% or lower, preferably determined at a concentration of 100 μg/mL.

In some embodiments, an antibody construct of the invention induces a degree of fratricide that is lower as compared to the anti-CD38 antibody shown in SEQ ID NOs: 167 and 168, preferably determined at a concentration of 100 μg/mL of the test antibody and the control.

In some embodiments, an antibody construct of the invention an antibody construct of the invention has a higher potency (lower EC50) in a cytotoxicity assay as compared to a reference antibody having only two or one first binding domains (A). A preferred reference antibody is preferably bivalent for the first target (A′) and bivalent for the second target (B′). A preferred reference antibody consists of a full-length immunoglobulin that is specific for the first target (A′) in which a scFv that is specific for the second target (B′) is fused to the C terminus of each heavy chain. As an illustrative example, such a reference antibody may have the heavy and light chain sequences set forth in SEQ ID NOs: 170 and 171. Alternatively, a preferred reference antibody consists of a full-length immunoglobulin that is specific for the second target (B′) in which a scFv that is specific for the first target (A′) is fused to the C terminus of each heavy chain. As an illustrative example, such a reference antibody may have the heavy and light chain sequences set forth in SEQ ID NOs: 176 and 177. The at least four first binding domains of the antibody of the invention preferably comprise the same CDR sequences as the binding domains specific for the first target (A′) of the reference antibody. Even more preferably, the at least four first binding domains of the antibody of the invention preferably comprise the same VL and VH sequences as the binding domains specific for the first target (A′) of the reference antibody. The potency (EC50) is preferably determined in a cytotoxicity assay as essentially described in Example 6. In some embodiments, the EC50 of the antibody construct of the invention has a numerical value that is about 0.5 times or lower as compared to the EC50 the reference antibody, preferably about 0.4 times or lower, preferably about 0.3 times or lower, preferably about 0.2 times or lower, preferably about 0.1 times. In principle, the potency can be determined with any target cell that expresses the second target (B′). However, the cell is preferably a tumor or cancer cell line. The target cell may have high expression of the second target (B′). In such a case, the EC50 of the antibody construct of the invention may have a numerical value that is about 0.5 times or lower as compared to the reference antibody. The increase in efficacy, however, become more pronounced when a target cell having low expression or even very low expression of the second target (B′) is used. In such a case, the EC50 of the antibody construct of the invention may have a numerical value that is about 0.5 times or lower as compared to the EC50 the reference antibody, preferably about 0.4 times or lower, preferably about 0.3 times or lower, preferably about 0.2 times or lower, more preferably about 0.1 times or lower as compared to the reference antibody.

There are several methods in the art to measure the expression level of a second target (B′) on a cell line. A preferred method according to the disclosure is the measurement of a specific antibody binding capacity (SABC). SACB assays are known in the art (Serke et al., 1998, Cytometry, 33 (2): 179-87). Such an assay may be conducted as essentially described in Example 5. In particular, the density of an antigen on the surface of one or more cell line can be determined using QIFIKIT (Dako) and suitable antibodies, such as anti-HER2 mAb MAB 1129 (RnD Systems) or anti-EGFR mAb H11 (Dianova), according to the manufacturer's instructions. In brief, aliquots of 1×10⁶ cells can be stained with a suitable antibody (such as mAb MAB 1129 or mAb H11) followed by F(ab′) 2 fragment of FITC-conjugate goat anti-mouse IgG. As negative control aliquots of 1×10⁶ cells can be stained with a negative control antibody (such as mAb 9E10 (Acris)) followed by F(ab′)2 fragment of FITC-conjugate goat anti-mouse IgG. To calculate the specific antibody binding capacity calibration beads containing 5 populations of beads bearing different distinct numbers of mAb molecules can be stained with F(ab′) 2 fragment of FITC-conjugate goat anti-mouse IgG. From the resulting median fluorescence intensities, a calibration curve can be generated. This calibration curve can be used to calculate the specific antibody binding capacity (SABC) for the respective antibody (such as mAb MAB 1129 or H11) of the respective cell line. High expressing cell lines for different second targets (B′) are described in the art. For EGFR, a high expressing cell line is A-431. For CD19, a high expressing cell line is JOK-1 (Reusch et al, 2015 MAbs, 7 (3): 584-604). For CD20 a high expressing cell line is DHL-10 (Watanabe et al., J Immunol Feb. 1, 2015, 194 (3) 911-920). For CD22, a high expressing cell line is JOK-1. For CD30, a high expressing cell line is HDLM-2 (Zhao et al, 2015, ASCO abstract 3050). For CD33, a high expressing cell line is MOLM-13 (Friedrich et al., 2014, Mol Cancer Ther 13 (6): 1549-1557). For CD52, a high expressing cell line is U-698 (human protein atlas). For CD70, a high expressing cell line is U-266 (human protein atlas). For CD74, a high expressing cell line is HDLM-2 (human protein atlas). For CD79b, a high expressing cell line is Daudi (Engelberts et al, 2020, EBioMedicine, vol. 52, 102625). For CD123, a high expressing cell line is MOLM-13. For CLL1, a high expressing cell line is FOL-1. For BCMA, a high expressing cell line is NCI-H929. For FCRH5, a high expressing cell line is U-698 (human protein atlas). For EGFRvIII, a high expressing cell line is DK-MG. For HER2, a high expressing cell line is SK-BR-3. For GD2, a high expressing cell line is T98G (Golinelli et al, 2020 Cancer Gene Therapy 27:558-570). Any of one of the aforementioned cell lines is a preferred reference cell line for a high expressing cell line for the respective second target. A cell line is preferably classified as high expressing cell line, if it has at least 50% of the SABC score for the respective second target (B′) as compared with the reference high expressing cell line. A cell line is preferably classified as low expressing cell line, if it has 15% or less of the SABC score for the respective second target (B′) as compared with the reference high expressing cell line. A cell line is preferably classified as very low expressing cell line, if it has 5% or less of the SABC score for the respective second target (B′) as compared with the reference high expressing cell line. Very low expressing cell lines are to be understood as a subgroup of low expressing cell lines. It is understood that low and very low expressing cell lines preferably still have detectable expression of the respective second target (B′) in the SABC assay.

In some embodiments, an antibody construct of the invention an antibody construct of the invention has a higher efficacy (higher Eₘₐₓ) in a cytotoxicity assay as compared to a reference antibody having only two or one first binding domains (A). A preferred reference antibody is preferably bivalent for the first target (A′) and bivalent for the second target (B′). A preferred reference antibody consists of a full-length immunoglobulin that is specific for the first target (A′) in which a scFv that is specific for the second target (B′) is fused to the C terminus of each heavy chain. As an illustrative example, such a reference antibody may have the heavy and light chain sequences set forth in SEQ ID NOs: 170 and 171. Alternatively, a preferred reference antibody consists of a full-length immunoglobulin that is specific for the second target (B′) in which a scFv that is specific for the first target (A′) is fused to the C terminus of each heavy chain. As an illustrative example, such a reference antibody may have the heavy and light chain sequences set forth in SEQ ID NOs: 176 and 177. The at least four first binding domains of the antibody of the invention preferably comprise the same CDR sequences as the binding domains specific for the first target (A′) of the reference antibody. Even more preferably, the at least four first binding domains of the antibody of the invention preferably comprise the same VL and VH sequences as the binding domains specific for the first target (A′) of the reference antibody. The efficacy (Eₘₐₓ) is preferably determined in a cytotoxicity assay as essentially described in Example 6.

The present invention also relates to a nucleic acid molecule (DNA and RNA) that includes nucleotide sequences encoding an antibody construct disclosed herein. The present disclosure also encompasses a vector comprising a nucleic acid molecule of the invention. The present invention also encompasses a host cell containing said nucleic acid molecule or said vector. Since the degeneracy of the genetic code permits substitutions of certain codons by other codons specifying the same amino acid, the disclosure is not limited to a specific nucleic acid molecule encoding an antibody construct as described herein but encompasses all nucleic acid molecules that include nucleotide sequences encoding a functional polypeptide. In this regard, the present disclosure also relates to nucleotide sequences encoding the antibody constructs of the disclosure.

A nucleic acid molecule disclosed in this application may be “operably linked” to a regulatory sequence (or regulatory sequences) to allow expression of this nucleic acid molecule.

A nucleic acid molecule, such as DNA, is referred to as “capable of expressing a nucleic acid molecule” or capable “to allow expression of a nucleotide sequence” if it includes sequence elements which contain information regarding to transcriptional and/or translational regulation, and such sequences are “operably linked” to the nucleotide sequence encoding the polypeptide. An operable linkage is a linkage in which the regulatory sequence elements and the sequence to be expressed are connected in a way that enables gene expression. The precise nature of the regulatory regions necessary for gene expression may vary among species, but in general these regions include a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the disclosure can include a regulatory sequence, such as a promoter sequence. In some embodiments a nucleic acid molecule of the disclosure includes a promoter sequence and a transcriptional termination sequence. Examples of promoters useful for expression in eukaryotic cells are the SV40 promoter or the CMV promoter.

The nucleic acid molecules of the disclosure can also be part of a vector or any other kind of cloning vehicle, such as a plasmid, a phagemid, a phage, a baculovirus, a cosmid or an artificial chromosome.

Such cloning vehicles can include, aside from the regulatory sequences described above and a nucleic acid sequence encoding a antibody construct as described herein, replication and control sequences derived from a species compatible with the host cell that is used for expression as well as selection markers conferring a selectable phenotype on transformed or transfected cells. Large numbers of suitable cloning vectors are known in the art, and are commercially available.

The disclosure also relates to a method for the production of an antibody construct of the disclosure, wherein the antibody construct is produced starting from the nucleic acid coding for the antibody construct or any subunit therein. The method can be carried out in vivo, the polypeptide can, for example, be produced in a bacterial or eukaryotic host organism and then isolated from this host organism or its culture. It is also possible to produce an antibody construct of the disclosure in vitro, for example by use of an in vitro translation system.

When producing the antibody construct in vivo, a nucleic acid encoding such polypeptide is introduced into a suitable bacterial or eukaryotic host organism by means of recombinant DNA technology. For this purpose, the host cell may be transformed with a cloning vector that includes a nucleic acid molecule encoding an antibody construct as described herein using established standard methods. The host cell may then be cultured under conditions, which allow expression of the heterologous DNA and thus the synthesis of the corresponding polypeptide or antibody construct. Subsequently, the polypeptide or antibody construct is recovered either from the cell or from the cultivation medium.

Suitable host cells can eukaryotic, such as immortalized mammalian cell lines (e.g., HeLa cells or CHO cells) or primary mammalian cells.

An antibody construct of the disclosure as described herein may be not necessarily generated or produced only by use of genetic engineering. Rather, such polypeptide can also be obtained by chemical synthesis such as Merrifield solid phase polypeptide synthesis or by in vitro transcription and translation. Methods for the solid phase and/or solution phase synthesis of proteins are well known in the art (see e.g. Bruckdorfer, T et al. (2004) Curr. Pharm. Biotechnol. 5, 29-43).

An antibody construct of the disclosure may be produced by in vitro transcription/translation employing well-established methods known to those skilled in the art.

The invention also provides a composition, preferably a pharmaceutical composition comprising an antibody construct of the invention.

Certain embodiments provide pharmaceutical compositions comprising the antibody construct defined in the context of the invention and further one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of one aspect of the invention to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18″ Edition, (A. R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine
antimicrobials such as antibacterial and antifungal agents
antioxidants such as ascorbic acid, methionine, sodium sulfite or sodium hydrogen-sulfite;
buffers, buffer systems and buffering agents which are used to maintain the composition at physiological pH or at a slightly lower pH; examples of buffers are borate, bicarbonate,
Tris-HCl, citrates, phosphates or other organic acids, succinate, phosphate, and histidine; for example Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetraacetic acid (EDTA);
isotonic and absorption delaying agents;
complexing agents such as caffeine, polyvinylpyrrolidone, beta-cyclodextrin or hydroxypropyl-beta-cyclodextrin)
fillers;
monosaccharides; disaccharides; and other carbohydrates (such as glucose, mannose or dextrins); carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as glycerin, propylene glycol or polyethylene glycol);
sugars and sugar alcohols, such as trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

It is evident to those skilled in the art that the different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles.

In one embodiment of the pharmaceutical composition according to one aspect of the invention the composition is administered to a patient intravenously.

Methods and protocols for the intravenous (iv) administration of pharmaceutical compositions described herein are well known in the art.

The antibody construct and/or pharmaceutical composition of the invention is preferably used in the prevention, treatment or amelioration of a disease, which his preferably selected from a proliferative disease, a tumorous disease, a viral disease and/or an immunological disorder. Preferably, said tumorous disease is a malignant disease, preferably cancer.

In one embodiment of the antibody construct and/or pharmaceutical composition of the invention, the identified malignant disease is selected from the group consisting of Hodgkin lymphoma, Non-Hodgkin lymphoma, leukemia, multiple myeloma and solid tumors.

According to the disclosure, the antibody construct and/or pharmaceutical composition of the invention is preferably for use in treating a tumor comprising cells that express the second target (B′). The expression of the second target (B′) within a tumor may be heterogenous. For example, the second target (B′) may be higher expressed in a certain tumor subtype or within the tumor tissue. However, despite heterogenous intratumoral expression, tumors can be classified into high or low expressors of a certain antigen, based on the overall expression in the tumor or tumor tissue. A preferred method such a classification is by immunohistochemistry.

The antibody construct of the invention can be used for the treatment any cancer tumor expressing the second target (B′), including a cancer or tumor with high expression of the second target or a cancer or tumor with low expression or very low expression of the second target, with the latter two being preferred. It is believed that the antibody construct of the invention is particularly advantageous for the treatment of cancers or tumors with low expression or very low expression of the second target, because of the at least four first binding domains, which are capable of activating an innate immune effector cell even at low or very low expression of the second target (B′). An antibody construct of the invention is further preferably for use in the treatment of a disease comprising malignant cells having low or very low expression of the second target (B′). Accordingly, the antibody construct of the invention may be for the use of treating cells with reduced expression of the second target (B′) (e.g. by downregulation or shedding) on tumor cells and/or cancer stem cells can otherwise lead to therapy resistance.

Since the antibody construct of the invention is not only effective for the treatment of cells that have a high expression of the second target (B′), but also for the treatment of cells that have a low or very low expression of the second target, the antibody construct may also be useful for the prevention of a relapse of the disease. Because the antibody construct of the invention may also be effective against low or very low expressing cells for the second target (B′), those cells may be effectively removed due to the treatment with the antibody construct of the invention. Otherwise, e.g. when using other therapies such as other antibody constructs, such low-expressing cells might escape the treatment with the other therapy, causing relapse of the disease. Thus, the antibody constructs of the invention may be for use in the treatment of a disease and/or the prevention of the relapse of the disease. In particular, the use in the treatment of the disease may comprise prevention of a relapse of the disease.

The present invention also provides a method for the treatment or amelioration of a disease, the method comprising the step of administering to a subject in need thereof an antibody construct according to the invention.

In one embodiment of said method for the treatment or amelioration of a disease the subject suffers from a proliferative disease, a tumorous disease, an infectious disease such as a viral disease, or an immunological disorder. It is preferred that said tumorous disease is a malignant disease, preferably cancer.

In one embodiment of said method for the treatment or amelioration of a disease said malignant disease is selected from the group consisting of Hodgkin lymphoma, Non-Hodgkin lymphoma, leukemia, multiple myeloma and solid tumors.

The present invention also relates to a method of simultaneously binding a target cell and an immune effector cell, comprising administering to a subject the antibody construct of the invention, wherein the target cell preferably has a low or very low expression of the second target (B′). Such a method preferably for the treatment or amelioration of a disease defined herein. Simultaneously binding of a target cell and an immune effector cell preferably comprises target cell specific activation of the immune effector cell.

The present invention also relates to a kit comprising an antibody construct of the invention, a nucleic acid molecule of the invention, a vector of the invention or a host cell of the invention. The kit of the invention will typically comprise a container comprising the antibody construct of the invention, the nucleic acid molecule of the invention, the vector of the invention, or the host cell of the invention, and optionally one or more other containers comprising materials desirable from a commercial and user standpoint, including buffers, diluents, filters, needles, syringes, and package inserts with instructions for use.

In some embodiments, the antibody constructs of the invention mediate (preferably concentration-dependent) lysis of target cells expressing low levels of the target antigen. For example, all EGFR/CD16A bispecific antibodies tested in the Examples mediated concentration-dependent lysis of target cells expressing low levels of EGFR (mean SABC determined for MCF-7:4546) and cells expressing very low levels of EGFR (mean SABC determined for Daudi: 868). Further, all EGFR/NKp46 bispecific antibodies tested in the examples mediated concentration-dependent lysis of A431 target cells expressing low levels of HER2 by NK cells.

In some embodiments, the antibody constructs of the invention with four first binding domains (A) exhibit higher potency and/or efficacy than constructs with two or one first binding domains (A). For example, all 3 constructs with 4 anti-Fv domains (Bi-scDb-Fc_02, aBi-scDb-Fc_05, and Bi-scDb-IgAb_06) tested in the Examples exhibited higher potency and efficacy than constructs with two or one anti-CD16A Fv domain. Among these constructs, aBi-scDb-Fc_05 with only one anti-EGFR domain, showed lower potency relative to the constructs with two anti-EGFR Fv domains. The IgG-based construct Bi-scDb-IgAb_06 exhibited slightly, but reproducible lower efficacy than Fc-based constructs suggesting that the longer distance between effector and target binding domains has a negative impact on the efficacy. Further, higher potency and efficacy were shown for constructs with 4 anti-NKp46 domains (AIG-2scDb_06) than construct with 2 anti-NKp46 domains (AIG-2scFv_27).

In some embodiment, on target cells expressing the second target (B′) the antibody constructs of the invention with four first binding domains (A) mediate concentration-dependent phagocytosis by macrophages with higher efficacy than antibody constructs having two first binding domains (A). The target cell may have high expression of the second target (B′). Alternatively, the target cell may have low expression of the second target (B′). On target cell expressing high levels of EGFR all tested EGFR/CD16A bispecific antibodies mediated concentration-dependent phagocytosis by macrophages with the constructs containing 4 anti-Fv domains (Bi-scDb-Fc_02, aBi-scDb-Fc_05, and Bi-scDb-IgAb_06) showing the highest efficacy. However, on target cells expressing low levels of EGFR only constructs with 4 anti-Fv domains (Bi-scDb-Fc_02, aBi-scDb-Fc_05, and Bi-scDb-IgAb_06) induced phagocytosis by macrophages to a similar level.

The invention is further characterized by the following items.

Item 1. An antibody construct comprising
(i.) at least four first binding domains (A), wherein said first binding domain (A) is capable of specifically binding to a first target (A′) that is an immune-regulatory antigen on the surface of an innate immune effector cell, wherein the immune effector cell is a natural killer cell or a macrophage; and
(ii.) a second binding domain (B), which is capable of specifically binding to a second target (B′) that is an antigen on the surface of a target cell.

Item 2. The antibody construct of item 1, wherein the antibody construct binds to a target cell and an immune effector cell simultaneously.

Item 3. The antibody of construct of item 1 or 2, wherein the first target (A′) is an immune activating antigen or an immune inhibitory antigen.

Item 4. The antibody construct of any one of the preceding items, wherein the first target (A′) is selected from the group consisting of CD16A, CD56, NKG2A, NKG2D, NKp30, NKp44, NKp46, NKp80, DNAM-1 (CD226), SLAMF7 (CD319), CD244 (2B4), OX40, CD47, SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5, KIR3DS1, and CD3.

Item 5. The antibody construct of any one of the preceding items, wherein the antibody construct is bispecific.

Item 6. The antibody construct of any one of items 1-4, wherein the antibody construct comprises a third binding domain (C), which is capable of specifically binding to a third target (C′) that is an antigen on the surface of a target cell that is other than the second target (B′).

Item 7. The antibody construct of any one of the preceding items, further comprising a fourth domain (D) comprising a half-life extension domain.

Item 8. The antibody construct of item 7, wherein said half-life extension domain comprises a CH2 domain, wherein the Fcγ receptor binding domain is silenced.

Item 9. The antibody construct of item 7 or 8, wherein said half-life extension domain comprises a CH3 domain.

Item 10. The antibody construct of any one of items 7 to 9, wherein the antibody construct comprises at least one hinge domain and CH3 domain fused to a CH2 domain in an amino to carboxyl order hinge-CH2 domain-CH3 domain.

Item 11. The antibody construct of any one of items 7 to 10, wherein the antibody construct comprises at least two of the hinge-CH2 domain-CH3 domain elements.

Item 12. The antibody construct of any one of the preceding items, wherein the second binding domain (B) comprises a VH and a VL domain of an antibody.

Item 13. The antibody construct of any one of the preceding items, wherein the second binding domain (B) is a Fab or an scFv.

Item 14. The antibody construct of any one of the preceding items, wherein the second target (B′) is selected from the group consisting of CD19, CD20, CD22, CD30, CD33, CD52, CD70, CD74, CD79b, CD123, CLL1, BCMA, FCRH5, EGFR, EGFRvlll, HER2, and GD2.

Item 15. The antibody construct of any one of items 6-14, wherein the third binding domain (C) comprises a VH and a VL domain of an antibody.

Item 16. The antibody construct of any one items 6-15, wherein the third binding domain (C) is a Fab or an scFv.

Item 17. The antibody construct of any one of items 6-16, wherein the third target (C′) is selected from the group consisting of CD19, CD20, CD22, CD30, CD33, CD52, CD70, CD74, CD79b, CD123, CLL1, BCMA, FCRH5, EGFR, EGFRvlll, HER2, and GD2.

Item 18. The antibody construct of any one of the preceding items, wherein the first binding domain (A) comprises a VH and a VL domain of an antibody.

Item 19. The antibody construct of any one of the preceding items, wherein the first target (A′) is CD16A.

Item 20. The antibody construct of any one of the preceding items, wherein the first binding domain (A) binds to an epitope on CD16A which is C-terminal to the physiological Fcγ receptor binding domain, said epitope preferably comprises Y158 of SEQ ID NO: 13.

Item 21. The antibody construct of any one of the preceding items, wherein the four binding domains (A) are positioned to each other in a way that simultaneous binding of two immune effector cells is reduced or preferably prevented.

Item 22. The antibody construct of any one of the preceding items, wherein the at least four first binding domains (A)
(a) comprise the same CDR sequences,
(b) comprise the same VL and VH sequences, and/or
(c) comprise the same amino acid sequence.

Item 23. The antibody construct of any one of the preceding items, wherein a first first binding domain (A1) and a second first binding domain (A2) of the four first binding domains (A) are fused to each other (A1A2) in form of a bi-scFv, double Fab, Db or scDb, preferably in form of a bi-scFv or scDb, preferably in form of a scDb, wherein the variable domains of the scDb are preferably arranged in V_{L}-V_{H}-V_{L}-V_{H} order.

Item 24. The antibody construct of any one of the preceding items, wherein a third first binding domain (A3) and a fourth first binding domain (A4) of the four first binding domains (A) are fused to each other (A3A4) in form of a bi-scFv, double Fab, Db or scDb, preferably in form of a bi-scFv or scDb, preferably in form of a scDb, wherein the variable domains of the scDb are preferably arranged in V_{L}-V_{H}-V_{L}-V_{H} order.

Item 25. The antibody construct of any one items 7 to 24, wherein a first first binding domain and a second first binding domain that are fused to each other (A1A2) is fused to the C terminus of a CH3 domain of a fourth domain (D).

Item 26. The antibody construct of any one of items 7 to 24, wherein a first first binding domain and a second first binding domain that are fused to each other (A1A2) is fused to the N terminus of a hinge of a fourth domain (D).

Item 27. The antibody construct of any one of items 7 to 24, wherein a third first binding domain and a fourth first binding domain that are fused to each other (A3A4) is fused to the C terminus of a CH3 domain of a fourth domain (D).

Item 28. The antibody construct of any one of items 7 to 24, wherein a third first binding domain and a fourth first binding domain that are fused to each other (A3A4) is fused to the N terminus of a hinge of a fourth domain (D).

Item 29. The antibody construct of any one of items 7 to 24, wherein a first first binding domain and a second first binding domain that are fused to each other (A1A2) is fused to the C terminus of a first CH3 domain of a fourth domain (D), and wherein a third first binding domain and a fourth first binding domain that are fused to each other (A3A4) is fused to the C terminus of a second CH3 domain of a fourth domain (D).

Item 30. The antibody construct of any one of items 7 to 24, wherein a first first binding domain and a second first binding domain that are fused to each other (A1A2) is fused to the N terminus of a first hinge of a fourth domain (D), and wherein a third first binding domain and a fourth first binding domain that are fused to each other (A3A4) is fused to the N terminus of a second hinge of a fourth domain (D).

Item 31. The antibody construct of any one of items 7 to 24, wherein a first first binding domain and a second first binding domain that are fused to each other (A1A2) is fused to the C terminus of a CH3 domain of a fourth domain (D), and wherein a third first binding domain and a fourth first binding domain that are fused to each other (A3A4) is fused to the N terminus of a hinge of a fourth domain (D).

Item 32. The antibody construct of any one of items 7 to 29 and 31, wherein a second binding domain (B) is fused to the N terminus of a hinge of a fourth domain (D).

Item 33. The antibody construct of any one of items 7 to 28 and 30 to 31, wherein a second binding domain (B) is fused to the C terminus of a CH3 domain of a fourth domain (D).

Item 34. The antibody construct of any one of items 7 to 25, 27, and 29, wherein a second binding domain (B) is fused to the N terminus of a hinge of a fourth domain (D), and wherein another second binding domain (B) is fused to the N terminus of another hinge of a fourth domain (D).

Item 35. The antibody construct of any one of items 7 to 24, 26, 28, and 30, wherein a second binding domain (B) is fused to the C terminus of a CH3 domain of a fourth domain (D), and wherein another second binding domain (B) is fused to the C terminus of another CH3 domain of a fourth domain (D).

Item 36. The antibody construct of any one of items 7 to 28 and 31, wherein a second binding domain (B) is fused to the N terminus of a hinge of a fourth domain (D), and wherein another second binding domain (B) is fused to the C terminus of a CH3 domain of a fourth domain (D).

Item 37. The antibody construct of any one of items 7 to 28 and 30 to 33, wherein a third binding domain (C) is fused to the C terminus of a CH3 domain of a fourth domain (D).

Item 38. The antibody construct of any one of items 7 to 29 and 31 to 33, wherein a third binding domain (C) is fused to the N terminus of a hinge of a fourth domain (D).

Item 39. The antibody construct of any one of items 7 to 25, 27, and 29, wherein a second binding domain (B) is fused to the N terminus of a hinge of a fourth domain (D), and wherein a third binding domain (C) is fused to the N terminus of another hinge of a fourth domain (D).

Item 40. The antibody construct of any one of items 7 to 24, 26, 28, and 30, wherein a second binding domain (B) is fused to the C terminus of a CH3 domain of a fourth domain (D), and wherein a third binding domain (C) is fused to the C terminus of another CH3 domain of a fourth domain (D).

Item 41. The antibody construct of any one of items 7 to 28 and 31, wherein a second binding domain (B) is fused to the N terminus of a hinge of a fourth domain (D), and wherein a third binding domain (C) is fused to the C terminus of a CH3 domain of a fourth domain (D).

Item 42. The antibody construct of any one of items 7 to 28 and 31, wherein a second binding domain (B) is fused to the C terminus of a CH3 domain of a fourth domain (D), and wherein a third binding domain (C) is fused to the N terminus of a hinge of a fourth domain (D).

Item 43. The antibody construct of any one of items 7 to 24, wherein a first first binding domain and a second first binding domain that are fused to each other (A1A2) is fused to the C terminus of a first CH3 domain of a fourth domain (D), and wherein a third first binding domain and a fourth first binding domain that are fused to each other (A3A4) is fused to the C terminus of a second CH3 domain of a fourth domain (D), and wherein a second binding domain (B) is fused to the N terminus of a hinge of a fourth domain (D).

Item 44. The antibody construct of item 43, wherein another second binding domain (B) is fused to the N terminus of another hinge of a fourth domain (D).

Item 45. The antibody construct of item 43, wherein a third binding domain (C) is fused to the N terminus of another hinge of a fourth domain (D).

Item 46. The antibody construct of any one of items 7 to 24, wherein a first first binding domain and a second first binding domain that are fused to each other (A1A2) is fused to the N terminus of a first hinge of a fourth domain (D), and wherein a third first binding domain and a fourth first binding domain that are fused to each other (A3A4) is fused to the N terminus of a second hinge of a fourth domain (D), and wherein a second binding domain (B) is fused to the C terminus of a CH3 domain of a fourth domain (D).

Item 47. The antibody construct of item 46, wherein another second binding domain (B) is fused to the C terminus of another CH3 domain of a fourth domain (D).

Item 48. The antibody construct of item 47, wherein a third binding domain (C) is fused to the C terminus of another CH3 domain of a fourth domain (D).

Item 49. The antibody construct of any one of items 7 to 24, wherein a first first binding domain and a second first binding domain that are fused to each other (A1A2) is fused to the N terminus of a first hinge-CH2-CH3 element of a fourth domain (D), and wherein a third first binding domain and a fourth first binding domain that are fused to each other (A3A4) is fused to the C terminus of a second hinge-CH2-CH3 element a fourth domain (D).

Item 50. The antibody construct of item 49, wherein a second binding domain (B) is fused to the C terminus of the first hinge-CH2-CH3 element of a fourth domain (D).

Item 51. The antibody construct of item 50, wherein another second binding domain (B) is fused to the N terminus of the second hinge-CH2-CH3 element of a fourth domain (D).

Item 52. The antibody construct of item 50, wherein a third binding domain (C) is fused to the N terminus of the second hinge-CH2-CH3 element of a fourth domain (D).

Item 53. The antibody construct of item 49, wherein a second binding domain (B) is fused to the N terminus of the second hinge-CH2-CH3 element of a fourth domain (D).

54. The antibody construct of item 53, and wherein a third binding domain (C) is fused to the C terminus of the first hinge-CH2-CH3 element of a fourth domain (D).

Item 55. The antibody construct of any one of the preceding items, wherein the first binding domain (A) comprises a VH region comprising CDR-H1, CDR-H2 and CDR-H3 and a VL region comprising CDR-L1, CDR-1.2 and CDR-L3 selected from:
(a) a CDR-H1 as depicted in SEQ ID NO: 77, a CDR-H2 as depicted in SEQ ID NO: 78, a CDR-H3 as depicted in SEQ ID NO: 79, a CDR-L1 as depicted in SEQ ID NO: 80, a CDR-L2 as depicted in SEQ ID NO: 81, and a CDR-L3 as depicted in SEQ ID NO: 82;
(b) a CDR-H1 as depicted in SEQ ID NO: 83, a CDR-H2 as depicted in SEQ ID NO: 84, a CDR-H3 as depicted in SEQ ID NO: 85, a CDR-L1 as depicted in SEQ ID NO: 86, a CDR-L2 as depicted in SEQ ID NO: 87, and a CDR-L3 as depicted in SEQ ID NO: 88; and
(c) a CDR-H1 as depicted in SEQ ID NO: 77, a CDR-H2 as depicted in SEQ ID NO: 89, a CDR-H3 as depicted in SEQ ID NO: 79, a CDR-L1 as depicted in SEQ ID NO: 80, a CDR-L2 as depicted in SEQ ID NO: 81, and a CDR-L3 as depicted in SEQ ID NO: 82.

Item 56. The antibody construct of any one of the preceding items, having an amino acid sequence selected from the group consisting of SEQ ID NOs: 148, 149, 150 and 151, 152 and 153, 154 and 155, 156 and 157, 158 and 159, 160 and 161, 162 and 163, 180-183, 190, and 191 and 192.

Item 57. The antibody construct of any one of the preceding items, wherein the antibody construct induces a lower degree of NK cell fratricide than a reference antibody having the heavy and light chain sequences of SEQ ID NO: 166 and 167 in a cytotoxicity assay.

Item 58. The antibody construct of any one of the preceding items, wherein the antibody construct induces 40% or less NK cell fratricide.

Item 59. The antibody construct of any one of the preceding items, wherein the antibody construct has a higher potency (lower EC50) in a cytotoxicity assay as compared to a reference antibody having only two or one first binding domains (A).

Item 60. The antibody construct of item 59, wherein the EC50 of the antibody construct has a numerical value that is about 0.5 times or lower as compared to the EC50 the reference antibody, preferably determined with a target cell having high expression of the second target (B′).

Item 61. The antibody construct of item 59, wherein the EC50 of the antibody construct has a numerical value that is about 0.1 times or lower as compared to the EC50 of the reference antibody, preferably determined with a target cell having low expression of the second target (B′).

Item 62. The antibody construct of any one of the preceding items, wherein the antibody construct has a higher efficacy (higher Eₘₐₓ) in a cytotoxicity assay as compared to a reference antibody having only two or one first binding domains (A).

Item 63. A nucleic acid molecule comprising a sequence encoding an antibody construct of any one of items 1 to 62.

Item 64. A vector comprising a nucleic acid molecule of item 63.

Item 65. A host cell comprising a nucleic acid molecule of item 63 or a vector of item 64.

Item 66. A method of producing an antibody construct of any one of items 1 to 62, said method comprising culturing a host cell of item 65 under conditions allowing the expression of the antibody construct of any one of items 1 to 62 and optionally recovering the produced antibody construct from the culture.

Item 67. A pharmaceutical composition comprising an antibody construct of any one of items 1 to 62, or produced by the method of item 66.

Item 68. An antibody construct of any one of items 1 to 62 for use in therapy.

Item 69. The antibody construct of any one of items 1 to 62, or produced by the method of item 65, for use in the prevention, treatment or amelioration of a disease selected from a proliferative disease, a tumorous disease, a viral disease or an immunological disorder.

Item 70. The antibody construct for the use of item 69, wherein the disease is cancer or tumor, preferably a cancer or tumor with low expression of the second target (B′).

Item 71. The antibody construct for the use of item 69 or 70, wherein the disease comprises malignant cells having a low expression of the second target (B′).

Item 72. The antibody construct for the use of any one of items 69-71, wherein the use prevents relapse of the disease.

Item 73. A method of treatment or amelioration of a proliferative disease, a tumorous disease, a viral disease or an immunological disorder, comprising the step of administering to a subject in need thereof the antibody construct of any one of items 1 to 62, or produced by the method of item 66.

Item 74. A method of simultaneously binding a target cell and an immune effector cell, comprising administering to a subject the antibody construct of any one of items 1 to 62, wherein the target cell has a low expression of the second target (B′).

Item 75. A kit comprising an antibody construct of any one of items 1 to 62, or produced by the method of item 66, a nucleic acid molecule of item 63, a vector of item 64, and/or a host cell of item 65.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 10%, preferably within 5%, more preferably within 2%, even more preferably within 1% of a given value or range (plus (+) or minus (−)). It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms. For example, the disclosure of the term “comprising” includes the disclosure of the terms “consisting essentially of” as well as the disclosure of the term “consisting of”.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended to limit the scope of the present invention in any way.

### EXAMPLES

### Example 1: Expression and Purification of Antibody Constructs

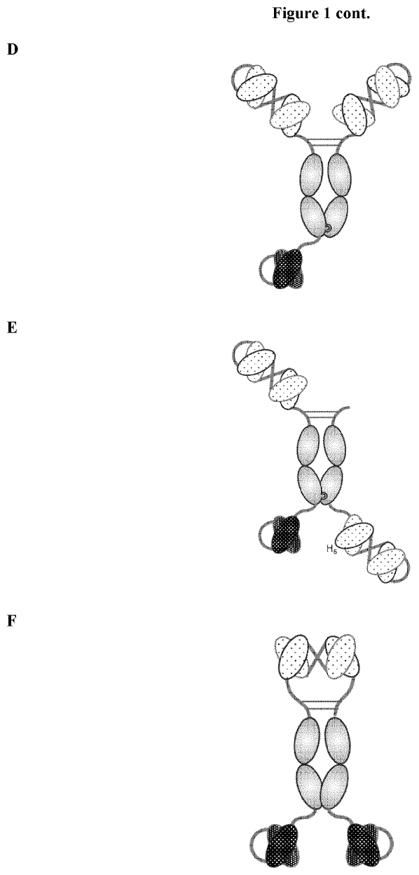

Stable expression of antibody constructs was performed as described by Ellwanger et al (MAbs. 2019 July; 11 (5): 899-918). Duplex body constructs were purified from clarified CHO cell culture supernatants in a two-step or three-step procedure comprising either Protein A, Protein L in combination with IMAC or C-Tag in combination with IMAC and then followed by preparative SEC, respectively. For Protein A, the clarified supernatant was loaded on a HiTrap MabSelectSuRe column. After washing with phosphate-buffered saline pH 7.4 and 10 mM sodium phosphate pH 7.0 protein was eluted in a two-step gradient with 10 mM sodium acetate pH 3.5 and 10 mM glycine/HCL pH 2.0. For Protein L, the clarified supernatant was loaded on a 5 mL HiTrap Protein L chromatography column. After washing with phosphate-buffered saline pH 7.4 and 10 mM sodium phosphate pH 7.0 protein was eluted in a two-step gradient with 10 mM glycine/HCl pH 3.0 and 10 mM glycine/HCl pH 2.0. For C-Tag, the clarified supernatant was loaded on a CaptureSelect C-tag XL column. After washing with phosphate-buffered saline pH 7.4 protein was eluted with 20 mM sodium citrate pH 3.0. For IMAC, target protein containing fractions were loaded onto a HisTrap FF chromatography column. After washing with IMAC A Buffer, the his-tagged target protein were eluted by sequential washing with 25% IMAC B Buffer and 100% IMAC B Buffer. The purity of fractions was analyzed using SE-HPLC and SDS-PAGE. Fractions exhibiting acceptable purity were pooled and subjected to preparative gel filtration using a Superdex 200 prep grade column. Eluate fractions containing purified duplex body constructs were pooled and subjected to buffer exchange using Sephadex G-25 column against 10 mM sodium acetate, 4.5% sorbitol pH 5.0, and concentrated by ultrafiltration. Final samples were assessed by SDS-PAGE under reducing and non-reducing conditions (see FIG. 2). The samples were mixed with nonreducing 2×SDS-PAGE sample buffer or reducing 2×SDS-PAGE sample buffer containing dithiothreitol (DTT) as reducing agent. All samples were heated at 95° C. for 5 min prior to loading on 4-20% Criterion TGX Precast SDS Page Gel. 2 μg of purified protein sample per lane were used. To separate the proteins in the gel, SDS-PAGE were run in 1×Tris/Glycine/SDS buffer at 300 V for approx. 22 min. Total protein were visualized in the gel using the Criterion Stain-free Molecular Imaging System (BioradBio-Rad). Page Ruler Unstained Protein ladder was used as molecular weight marker. The purity (Bi-scDb-IgAb_06 (SEQ ID NOs: 162 and 163) approx. 93%, Bi-scDb-Fc_01 (SEQ ID NOs: 148) approx. 92%, Bi-scDb-Fc_02 (SEQ ID NOs: 149) approx. 94%, aBi-scDb-Fc_01 (SEQ ID NOs: 150 and 151) approx. 98%, aBi-scDb-Fc_02 (SEQ ID NOs: 152 and 153) approx. 98%, aBi-scDb-Fc_03 (SEQ ID NOs: 154 and 155) approx. 98%, aBi-scDb-Fc_04 (SEQ ID NOs: 156 and 157) approx. 97%, aBi-scDb-Fc_05 (SEQ ID NOs: 158 and 159) approx. 96%, aBi-scDb-Fc_06 (SEQ ID NOs: 160 and 161) approx. 96%) were evaluated by analytical SE-HPLC using Superdex 200 Increase 10/300GL column. Purified proteins were stored as aliquots at −80° C. until further use.

### Example 2: Isolation of Peripheral Blood Mononuclear Cells (PBMC) from Buffy Coats

PBMCs were isolated from buffy coats by density gradient centrifugation. The buffy coat sample was diluted with a two-to-threefold volume of PBS, layered on a cushion of Lymphoprep (Stem Cell Technologies, cat.: 7861) and centrifuged at 800×g for 25 min at room temperature w/o brake. PBMC located in the interface were collected and washed 3 times with PBS before they were used for the enrichment of PBMC subsets or flow cytometric analysis. In most cases PBMC were cultured O/N in RPMI 1640 medium supplemented with 10% heat-inactivated FCS, 2 mM L-glutamine, 100 U/mL penicillin G sodium, and 100 μg/mL streptomycin sulfate, herein referred to as complete RPMI 1640 medium, at 37° C. and 5% CO₂ in a humidified atmosphere before they were used for the enrichment of NK cells.

### Example 3: Enrichment of NK Cells from Human PBMC and Differentiation of Macrophages

NK cells were enriched from PBMC using Easy Sep™ NK enrichment kit (Stem Cell Technologies, cat.: 17955) for the immunomagnetic isolation of untouched human NK cells according to the manufacturer's instructions. The purity of NK cell isolation was determined by flow cytometry. For macrophage differentiation, PBMC were discarded after overnight culture while adherent mononuclear cells were used for subsequent differentiation protocol. Complete RPMI 1640 medium supplemented with human M-CSF (50 ng/mL final) was added to monocytes and replenished every 5-6 days. Depending on cell morphology, density and growth, adherent macrophages were harvested after 1-4 weeks using accutase treatment for subsequent analyses.

### Example 4: Culture of Tumor Cell Lines

The MCF-7 cell line was purchased from DSMZ (cat.: ACC 115), and cultured under standard conditions in RPMI 1640 medium supplemented with 10% heat-inactivated FCS, 2 mM L-glutamine, 100 U/mL penicillin G sodium, 100 μg/mL streptomycin sulfate, and 1 mM sodium pyruvate as recommended by the supplier at 37° C. and 5% CO₂ in a humidified atmosphere.

The Daudi cell line was purchased from DSMZ (cat.: ACC 78), and cultured under standard conditions in RPMI 1640 medium supplemented with 10% heat-inactivated FCS, 2 mM L-glutamine, 100 U/mL penicillin G sodium, and 100 μg/mL streptomycin sulfate as recommended by the supplier at 37° C. and 5% CO₂ in a humidified atmosphere.

The MM.1S cell line was purchased from ATCC (cat.: CRL-2974), and cultured under standard conditions in RPMI 1640 medium supplemented with 10% heat-inactivated FCS, 2 mM L-glutamine, 100 U/mL penicillin G sodium, and 100 μg/mL streptomycin sulfate as recommended by the supplier at 37° C. and 5% CO₂ in a humidified atmosphere.

The DK-MG cell line was purchased from DSMZ (cat.: ACC 277), and cultured under standard conditions in RPMI 1640 medium supplemented with 10% heat-inactivated FCS, 2 mM L-glutamine, 100 U/mL penicillin G sodium, and 100 μg/mL streptomycin sulfate as recommended by the supplier at 37° C. and 5% CO₂ in a humidified atmosphere.

The HCT-116 cell line was purchased from DSMZ (cat.: ACC 581), and cultured under standard conditions in RPMI 1640 medium supplemented with 10% heat-inactivated FCS, 2 mM L-glutamine, 100 U/mL penicillin G sodium, 100 μg/mL streptomycin sulfate, as recommended by the supplier at 37° C. and 5% CO₂ in a humidified atmosphere.

### Example 5: Quantification of Specific Antibody Binding Capacity as a Measure of Antigen Expression Level on Tumor Cell Lines

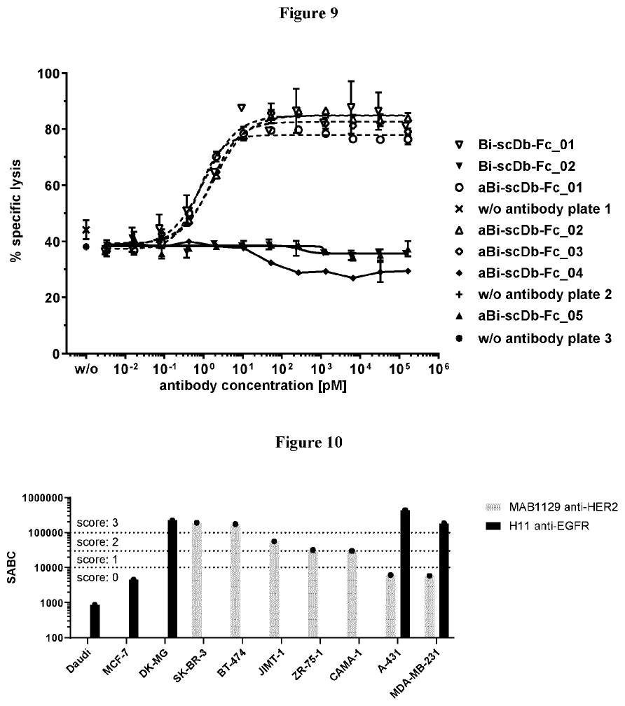

The density of HER2 and EGFR on the surface of different cell lines was determined using QIFIKIT (Dako) and anti-HER2 mAb MAB 1129 (RnD Systems) or anti-EGFR mAb H11 (Dianova) according to the manufacturer's instructions. In brief, aliquots of 1×10⁶ cells were stained with mAb MAB 1129 or mAb H11 followed by F(ab′)₂ fragment of FITC-conjugate goat anti-mouse IgG. As negative control aliquots of 1×10⁶ cells were stained with mAb 9E10 (Acris) followed by F(ab′)₂ fragment of FITC-conjugate goat anti-mouse IgG. To calculate the specific antibody binding capacity calibration beads containing 5 populations of beads bearing different distinct numbers of mAb molecules were stained with F(ab′)₂ fragment of FITC-conjugate goat anti-mouse IgG. From the resulting median fluorescence intensities a calibration curve was generated. This calibration curve was used to calculate the specific antibody binding capacity (SABC) for mAb MAB 1129 and H11 of the different cell lines. The HER2 and EGFR densities (SABC) depicted in Table 1 represent mean values of at least 2 independent experiments. SABC values determined with anti-HER2 mAb MAB 1129 and HER2 IHC score were used to generate an artificial EGFR score based on the SABC values determined with anti-EGFR mAb H11 (Table 1). Scoring of tumor cell lines regarding HER2 and EGFR expression based on specific antibody binding capacity (SABC) is illustrated in FIG. 10.

TABLE 1
Specific antibody binding capacity (SABC) determined with
anti-HER2 mAb MAB 1129 and anti-EGFR mAb H11 on various
tumor cell lines and scoring of expression levels.

| | mean HER2 | | | |
|---|---|---|---|---|
| | SABC (MAB | | mean EGFR | artificial EGFR |
| cell line | 1129) | HER2 IHC score | SABC (H11) | score |
| Daudi | n.d. | n.d. | 868 | 0 |
| MCF-7 | n.d. | 0 | 4,546 | 0 |
| DK-MG | n.d. | n.d. | 222,648 | 3 |
| SK-BR-3 | 191,321 | 3 | n.d. | n.d. |
| BT-474 | 174,805 | 3 | n.d. | n.d. |
| JIMT-1 | 56,485 | 2 | n.d. | n.d. |
| ZR-75-1 | 31,448 | 2 | n.d. | n.d. |
| CAMA-1 | 29,768 | 1 | n.d. | n.d. |
| A-431 | 6,150 | n.d. | 431,125 | 3 |
| MDA-MB-231 | 5,836 | 0 | 181,487 | 3 |

n.d., not determined.

### Example 6: Calcein-Release Cytotoxicity Assays on Tumor Target Cells with NK Cells as Effector Cells (E:T=5:1) in the Presence of Increasing Concentrations of Different Antibody Constructs

For the calcein-release cytotoxicity assays, target cells were labeled with 10 μM of the fluorescent dye calcein AM (Invitrogen, cat.: C3100MP) for 30 min, and aliquots of 1×10⁴ labeled target cells were seeded in individual wells of a round-bottom 96-well micro plate together with freshly isolated and enriched primary human NK cells at an effector:target (E:T) ratio of 5:1 in the presence of 12 serial 1:5 dilutions of the indicated antibodies usually starting at 30 μg/mL, if not otherwise indicated, in duplicates. Control samples to measure spontaneous release, maximal release and antibody-independent lysis by effector cells were tested in 4 replicates. After incubation for 4 h 100 μL cell-free culture supernatant was harvested from each well to quantify the fluorescent calcein released from lysed target cells with a multiplate fluorescence reader. After subtracting the fluorescence of spontaneously lysed cells from all samples, the fluorescence of each sample was normalized to the fluorescence of fully lysed cells to determine the specific lysis for a respective sample. Mean values of specific target cell lysis (%) and standard deviations (SD) were plotted and in vitro potency (EC₅₀) and efficacy (Eₘₐₓ) were determined by fitting the non-linear regression model to sigmoidal dose-response curves (variable slope) using GraphPad Prism (v6 and v7: GraphPad Software, La Jolla California USA).

### Example 7:4 h Calcein-Release Assays to Assess NK Cell Fratricide Induced by Increasing Concentrations of Various Antibody Constructs

For the calcein-release NK cell fratricide assays, enriched primary human NK cells were labeled with 10 μM of the fluorescent dye calcein AM for 30 min, and aliquots of 5×10⁴ labeled cells were seeded in individual wells of a round-bottom 96-well micro plate together with unlabeled, enriched autologous NK cells at an effector:target (E:T) ratio of 1:1 in the presence of 10 serial 1:5 dilutions of the indicated antibodies starting at 100 μg/mL in duplicates. Anti-CD38 IgG1 with daratumumab-derived Fab domains (IgAb 51, SEQ ID NOs: 166 and 167) was used as a positive control. Control samples to measure spontaneous release, maximal release and antibody-independent lysis by effector cells were tested in 4 replicates. After incubation for 4 h, 100 μL cell-free culture supernatant was harvested from each well to quantify the fluorescent calcein released from lysed target cells with a multiplate fluorescence reader. After subtracting the fluorescence of spontaneously lysed cells from all samples, the fluorescence of each sample was normalized to the fluorescence of fully lysed cells to determine the specific lysis for a respective sample. Mean values of specific target cell lysis (%) and standard deviations (SD) were plotted and in vitro potency (EC₅₀) and efficacy (Eₘₐₓ) were determined by fitting the non-linear regression model to sigmoidal dose-response curves (variable slope) using GraphPad Prism (v6 and v7; GraphPad Software, La Jolla California USA).

### Example 8: Phagocytosis Assays on Tumor Target Cells with Macrophages as Effector Cells (E:T=5:1) in the Presence of Increasing Concentrations of Different Antibody Constructs

For the phagocytosis assays, macrophages were seeded in 96-well UpCell plates and cultured overnight. Target cells were labeled with 0.5 μM CellTracker™ Green CMFDA Dye at 37° C. for 30 min, washed, and cultured overnight. Target cells were seeded on top of the macrophages (E:T ratio of 5:1), and the indicated antibodies were added at serial concentrations (0.3 pg/mL-30 μg/mL) in duplicates. After 4 hours incubation, cells were detached from the culture plate by incubation on ice and stained with A700-labeled anti-CD11b and fixable viability dye eF780 for 30 min at 4° C. Phagocytosis of labeled target cells was quantified by analyzing CMFDA⁺/CD11b⁺ cells in % of viable cells by flow cytometry. ADCP in absence of antibodies was used for normalization. Additionally, loss of labeled target cells was quantified by analyzing CMFDA⁺/CD11b⁻ cells in % of viable cells by flow cytometry. Loss of target cells in absence of antibodies was used for normalization.

### Example 9: Potency and Efficacy of EGFR-Targeting Innate Cell Engagers in 4 Cytotoxicity Assays on MCF-7 Target Cells Expressing Low EGFR Levels

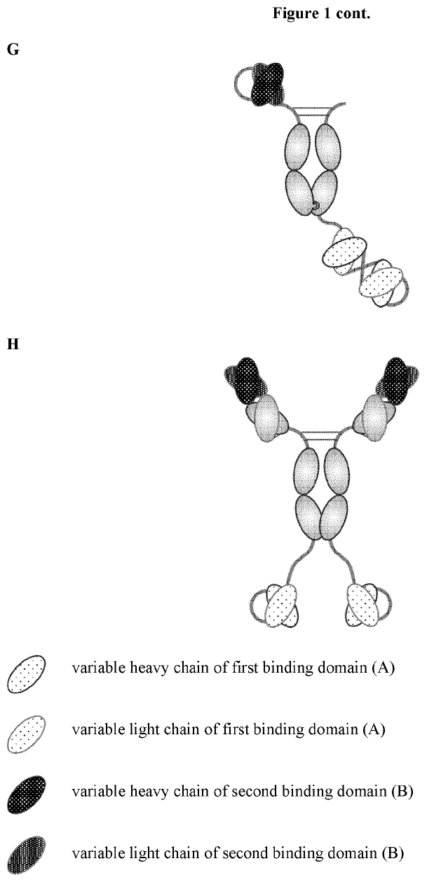

To compare the in vitro ADCC activity of multivalent anti-CD16A innate cell engagers targeting EGFR (Bi-scDb-Fc_02 (SEQ ID NOs: 149), aBi-scDb-Fc_05 (SEQ ID NOs: 158 and 159), Bi-scDb-IgAb_06 (SEQ ID NOs: 162 and 163)) with bivalent anti-EGFR engagers (scFv-IgAb_43 (SEQ ID NOs: 174 and 175), scFv-IgAb_167 (SEQ ID NOs: 178 and 179)), and with Fc-enhanced (S239D/1332E) anti-EGFR IgG1 (IgAb_53 (SEQ ID NOs: 168 and 169)), 4 h calcein-release cytotoxicity assays were performed as described in Example 6] on MCF-7 target cells expressing low levels of EGFR (mean SABC: 4,546) using primary enriched human NK cells as effector cells at an ET ratio of 5:1. The results summarized in Table 2 and the exemplary graph in FIG. 3 clearly demonstrate superior potency and efficacy of multivalent innate cell engagers with four anti-CD16A Fv domains relative to bivalent anti-CD16A constructs (scFv-IgAb) or Fc-enhanced anti-EGFR IgG1. Among the multivalent engagers, constructs with two anti-EGFR Fv domains exhibited approximately 2.5-fold higher potency than the construct aBi-scDb-Fc_05 with only one anti-EGFR Fv domain. In summary, these results show that multivalent CD16A engagement is not only advantageous regarding potency of ADCC-inducing innate cell engagers, but also regarding efficacy in target cell lysis mediated by NK cells.

TABLE 2
Potency and efficacy of anti-EGFR antibody constructs determined
in cytotoxicity assays on MCF-7 target cells.

| Construct | EC₅₀ [pM] | Eₘₐₓ [%] |
|---|---|---|
| Bi-scDb-Fc_02 | 1.9 | 32.9 |
| (SEQ ID NOs: 149) | | |
| aBi-scDb-Fc_05 | 5.0 | 34.2 |
| (SEQ ID NOs: 158 and 159) | | |
| Bi-scDb-IgAb_06 | 2.1 | 25.0 |
| (SEQ ID NOs: 162 and 163) | | |
| scFv-IgAb_43 | 9.2 | 16.2 |
| (SEQ ID NOs: 174 and 175) | | |
| scFv-IgAb_167 | 8.8 | 17.9 |
| (SEQ ID NOs: 178 and 179) | | |
| IgAb_53 | 100.5 | 16.1 |
| (SEQ ID NOs: 168 and 169) | | |

Potency (EC₅₀) and efficacy (Eₘₐₓ) were determined in 4 calcein-release cytotoxicity assays on MCF-7 target cells using NK cells as effector cells at an E:T ratio of 5:1. Mean values of three independent experiments are shown.

### Example 10: Potency and Efficacy of EGFR-Targeting Innate Cell Engagers in 4 h Cytotoxicity Assays on Daudi Target Cells Expressing Very Low EGFR Levels

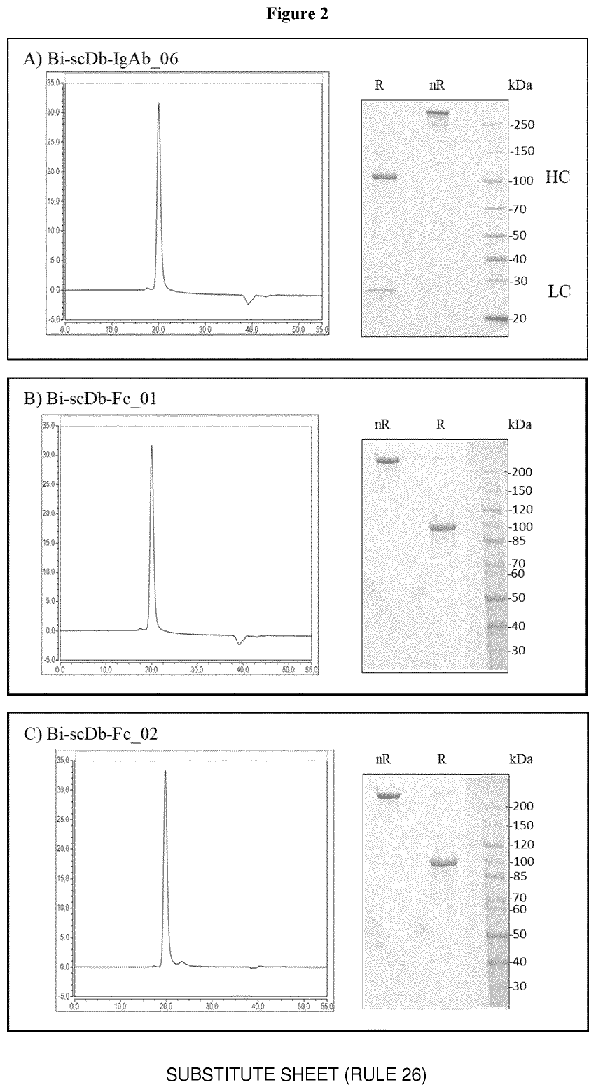

To assess the impact of multivalent CD16A engagement on the ADCC activity of EGFR-targeting innate cell engagers, 4 h calcein-release cytotoxicity assays were performed on Daudi target cells expressing very low levels of EGFR (mean SABC: 868) and enriched primary human NK cells as effector cells at an E:T ratio of 5:1. The assays performed as described in Example 6 were used to compare multivalent anti-CD16A constructs (Bi-scDb-Fc_02 (SEQ ID NOs: 149), aBi-scDb-Fc_05 (SEQ ID NOs: 158 and 159), Bi-scDb-IgAb_06 (SEQ ID NOs: 162 and 163)) with constructs comprising two anti-CD16A domains (scFv-IgAb_43 (SEQ ID NOs: 174 and 175) and scFv-IgAb_167 (SEQ ID NOs: 178 and 179)), and with Fc-enhanced (S239D/1332E) anti-EGFR IgG1 (IgAb_53 (SEQ ID NOs: 168 and 169)) in three independent assays using NK cells from different blood donors. The results summarized in Table 3 and in the exemplary graph in FIG. 4 demonstrate superior potency and efficacy of multivalent anti-CD16A constructs relative to bivalent anti-CD16A scFv-IgAb constructs or Fc-enhanced anti-EGFR IgG1. Notably, aBi-scDb-Fc_05 with only one Fv domain for EGFR exhibits approximately twofold lower potency relative to the other constructs comprising 4 anti-CD16A domains and two anti-EGFR domains. These results clearly show the advantage of multivalent anti-CD16A engagement for ADCC-mediating innate cell engagers, and bivalent tumor-targeting.

TABLE 3
Potency and efficacy of anti-EGFR antibody constructs determined
in cytotoxicity assays on Daudi target cells.

| Construct | EC₅₀ [pM] | Eₘₐₓ [%] |
|---|---|---|
| Bi-scDb-Fc_02 | 20.5 | 32.6 |
| (SEQ ID NOs: 149) | | |
| aBi-scDb-Fc_05 | 39.9 | 27.2 |
| (SEQ ID NOs: 158 and 159) | | |
| Bi-scDb-IgAb_06 | 18.6 | 18.2 |
| (SEQ ID NOs: 162 and 163) | | |
| scFv-IgAb_43 | 150.7 | 10.0 |
| (SEQ ID NOs: 174 and 175) | | |
| scFv-IgAb_167 | 46.8 | 8.0 |
| (SEQ ID NOs: 178 and 179) | | |
| IgAb_53 | 1075.8 | 9.1 |
| (SEQ ID NOs: 168 and 169) | | |

Potency (EC₅₀) and efficacy (Eₘₐₓ) were determined in 4 calcein-release cytotoxicity assays on Daudi target cells using NK cells as effector cells at an E:T ratio of 5:1. Mean values of three independent experiments are shown.

### Example 11: Assessment of NK Cell Fratricide Mediated by Multivalent Anti-CD16A Engagers in 4 h Calcein-Release Cytotoxicity Assays

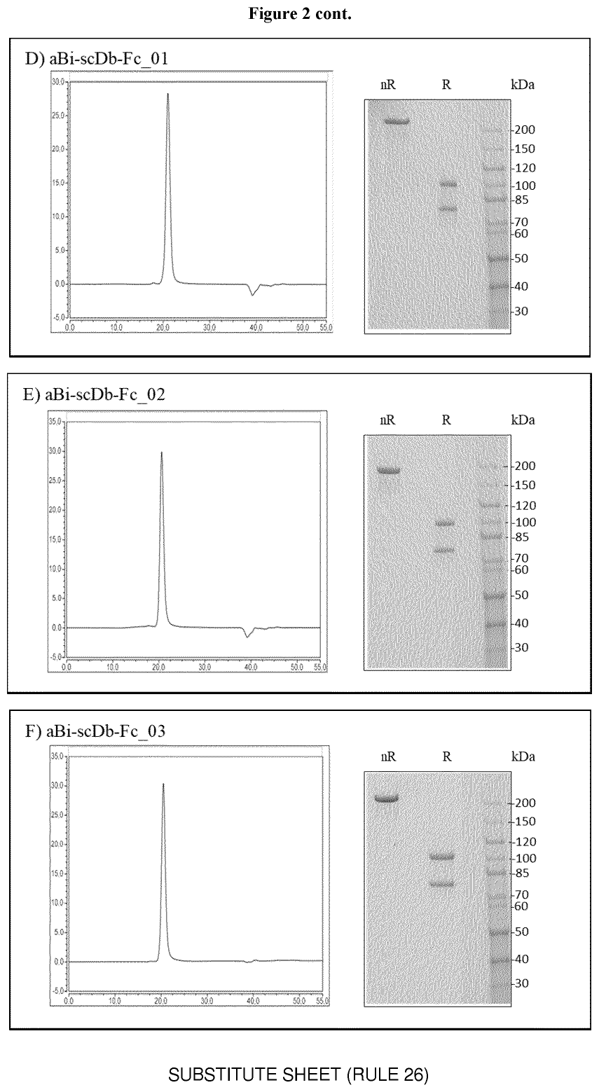

To evaluate whether multivalent anti-CD16A innate cell engagers targeting EGFR have the capability to cross-link NK cells with other NK cells and thereby induce NK cell fratricide, 4 h calcein-release assays using enriched primary human NK cells as target cells and autologous NK cells as effector cells as described in Example 7. The summary of independent assays in Table 4 and the exemplary graph in FIG. 5 show potent and efficacious NK cell fratricide induced by anti-CD38 IgG1 (IgAb_51 (SEQ ID NOs: 166 and 167)) with daratumumab-derived Fab domains used as a positive control. Fc-enhanced anti-EGFR IgG1 (IgAb_53 (SEQ ID NOs: 168 and 169)) and scFv-IgAb (scFv-IgAb_43 (SEQ ID NOs: 174 and 175) and scFv-IgAb_167 (SEQ ID NOs: 178 and 179)) induced no or only minimal NK-NK cell lysis (mean Eₘₐₓ: <7%). Multivalent innate cell engagers containing four anti-CD16A Fv domains induced NK cell fratricide with slightly higher efficacy. However, relative to the anti-CD38 IgG1, the multivalent anti-CD16A engagers induced only low NK cells fratricide (mean Eₘₐₓ: <30%) and only at higher antibody concentrations (>100 pM).

TABLE 4
Potency and efficacy of anti-EGFR antibody constructs
determined in NK cell fratricide assays.

| Construct | EC₅₀ [pM] | Eₘₐₓ [%] |
|---|---|---|
| Bi-scDb-Fc_02 | n.a. | 23.0 |
| (SEQ ID NOs: 149) | | |
| aBi-scDb-Fc_05 | ^{§}n.a. | ^{§}27.1 |
| (SEQ ID NOs: 158 and 159) | | |
| Bi-scDb-IgAb_06 | n.a. | 15.0 |
| (SEQ ID NOs: 162 and 163) | | |
| scFv-IgAb_43 | 3512.5 | 6.9 |
| (SEQ ID NOs: 174 and 175) | | |
| scFv-IgAb_167 | n.a. | 2.8 |
| (SEQ ID NOs: 178 and 179) | | |
| IgAb_53 | n.a. | 4.8 |
| (SEQ ID NOs: 168 and 169) | | |
| IgAb_51 | 66.5 | 68.6 |
| (SEQ ID NOs: 166 and 167) | | |

Potency (EC₅₀) and efficacy (Eₘₐₓ) were determined in 4 calcein-release NK cell fratricide assays using enriched primary human NK cells as target cells and effector cells at an E:T ratio of 1:1. Mean values of three independent experiments are shown.
n.a., not applicable;
^{§}determined in two independent assays.

### Example 12: 4h Phagocytosis Assay on DK-MG Cells

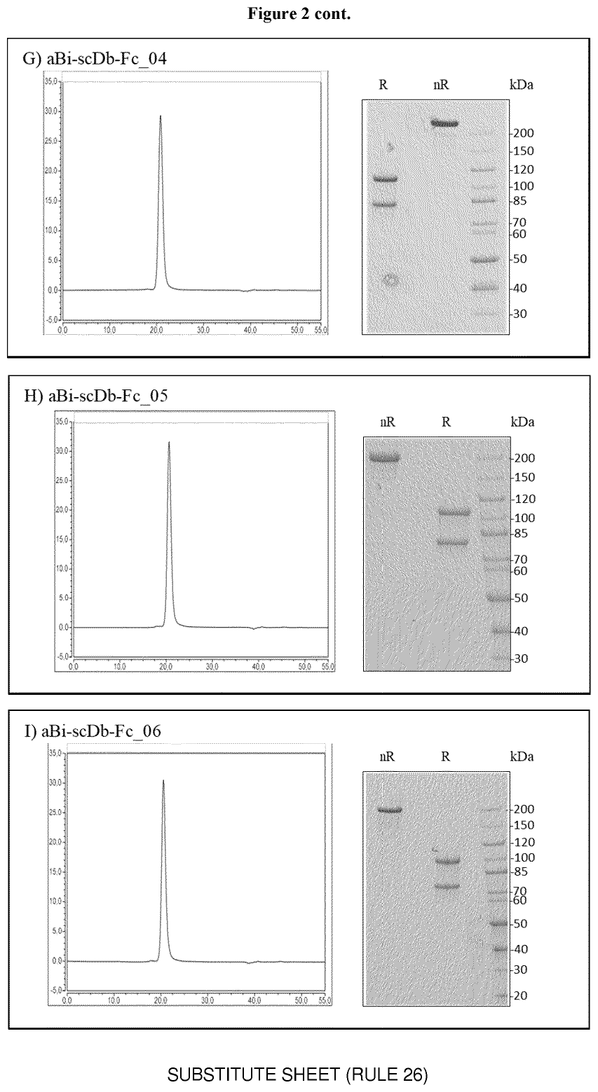

To assess the impact of multivalent CD16A engagement on the ADCP activity of EGFR-targeting innate cell engagers, 4 h phagocytosis assays were performed on DK-MG target cells expressing high levels of EGFR (mean SABC: 222648) and monocyte-derived human macrophages as effector cells at an ET ratio of 5:1. The assays performed as described in Example 8 were used to compare multivalent anti-CD16A constructs (Bi-scDb-Fc_02 (SEQ ID NOs: 149), aBi-scDb-Fc_05 (SEQ ID NOs: 158 and 159), Bi-scDb-IgAb_06 (SEQ ID NOs: 162 and 163)) with constructs comprising two anti-CD16A domains (scFv-IgAb_43 (SEQ ID NOs: 174 and 175)), with Fc-wildtype anti-EGFR IgG1 (IgAb_49 (SEQ ID NOs: 164 and 165)) and with Fc-enhanced (S239D/1332E) anti-EGFR IgG1 (IgAb_53 (SEQ ID NOs: 168 and 169)) in three independent assays using macrophages generated from monocytes of different blood donors. A representative exemplary graph in FIG. 6 demonstrates higher phagocytosis induction of multivalent anti-CD16A constructs relative to bivalent anti-CD16A scFv-IgAb construct, Fc-wildtype or Fc-enhanced anti-EGFR IgG1 antibodies. The lowest phagocytosis was induced by IgAb_49 and Fc-enhanced anti-EGFR IgG (IgAb_53). These results clearly show the advantage of multivalent anti-CD16A engagement for ADCP-mediating innate cell engagers.

### Example 13: 4h Phagocytosis Assay on MCF-7 Cells

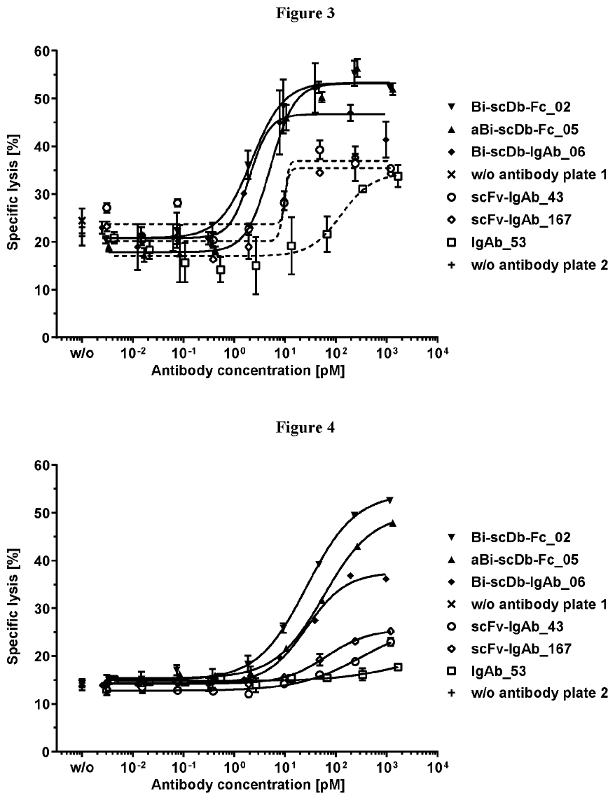

To assess the impact of multivalent CD16A engagement on the ADCP activity of EGFR-targeting innate cell engagers, 4 h phagocytosis assays were performed on MCF-7 target cells expressing low levels of EGFR (mean SABC: 4,546) and monocyte-derived human macrophages as effector cells at an ET ratio of 5:1. The assays performed as described in Example 8 were used to compare multivalent anti-CD16A constructs (Bi-scDb-Fc_02 (SEQ ID NOs: 149), aBi-scDb-Fc_05 (SEQ ID NOs: 158 and 159), Bi-scDb-IgAb_06 (SEQ ID NOs: 162 and 163)) with constructs comprising two anti-CD16A domains (scFv-IgAb_43 (SEQ ID NOs: 174 and 175)), with Fc-wildtype anti-EGFR IgG1 (IgAb_49 (SEQ ID NOs: 164 and 165)) and with Fc-enhanced (S239D/1332E) anti-EGFR IgG1 (IgAb_53 (SEQ ID NOs: 168 and 169)) in three independent assays using macrophages generated from monocytes of different blood donors. A representative exemplary graph in FIG. 7 demonstrates phagocytosis induction of multivalent anti-CD16A constructs. In contrast, scFv-IgAb_43, IgAb_49 and IgAb_53 did not induce phagocytosis of MCF-7 target cells by macrophages. These results clearly show the advantage of multivalent anti-CD16A engagement for ADCP-mediating innate cell engagers.

### Example 14:4 h Calcein-Release Assays on A-431

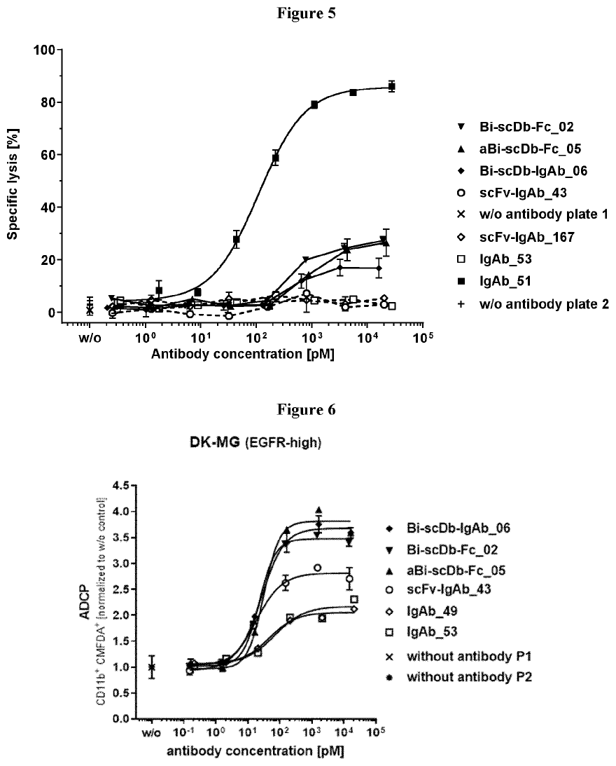

To compare the in vitro ADCC activity of multivalent anti-NKp46 innate cell engagers targeting EGFR (AIG-2scDb_06 (SEQ ID NOs: 180-183)) with bivalent anti-HER2 engagers (AIG-2scFv_27 (SEQ ID NOs: 184-187)), 4 h calcein-release cytotoxicity assays were performed as described in Example 6 on A-431 target cells expressing low levels of HER2 (mean SABC: 6,150) using primary enriched human NK cells as effector cells at an E:T ratio of 5:1. A representative graph in FIG. 8 clearly demonstrate superior potency and efficacy of multivalent innate cell engagers with four anti-NKp46 Fv domains relative to bivalent anti-NKp46 constructs. In summary, these results show that multivalent NKp46 engagement is not only advantageous regarding potency of ADCC-inducing innate cell engagers, but also regarding efficacy in target cell lysis mediated by NK cells.

### Example 15: ELISA Investigation of Binding of EGFR/CD16A Engagers or BCMA/CD16A Engagers to CD16A

To assess binding of EGFR/CD16 or BCMA/CD16 antibodies to coated antigen in ELISA, 96-well ELISA plates (Immuno Maxisorp, Nunc) were coated overnight at 4° C. with recombinant CD16 antigen variants fused to monomeric human Fc at a concentration of 1.5 μg/mL in 100 mM carbonate-bicarbonate buffer. After blocking with 3% (w/v) skimmed milk powder dissolved in phosphate buffered saline (PBS) serial dilutions of bispecific antibody constructs were incubated on the antigen coated plates for 1.5 h at room temperature. After washing three times with 300 μL per well of PBS containing 0.1% (v/v) Tween 20, plates were incubated with anti-AFM24 mAb 62-1-1 at 5 μg/mL for 1 h followed by washing and detection with peroxidase-conjugated goat anti-mouse IgG (H+L)-HRPO, MinX Hu,Bo,Ho 1:10,000 diluted in PBS containing 0.3% (w/v) skimmed milk powder. After washing, plates were incubated with tetramethylbenzidine substrate (Seramun) for 1-2 min. Reaction was stopped by addition of 0.5 M H₂SO₄ (100 μL/well). Absorbance was measured at 450 nm using a multiwell plate reader, plotted and EC₅₀ values were determined by fitting a nonlinear regression model to sigmoidal dose-response curves (four parameters logistic fit) using GraphPad Prism software.

To assess binding of soluble CD16A antigen to coated or antigen captured EGFR/CD16 or BCMA/CD16 antibodies, 96-well ELISA plates (Immuno Maxisorp, Nunc) were coated overnight at 4° C. with different multivalent bispecific engagers at concentrations of 3.5-5 μg/mL (equalizing 24-27 nM) in 100 mM Carbonate-bicarbonate buffer. For the capturing approach, 96-well ELISA plates (Immuno Maxisorp, Nunc) were coated overnight at 4° C. with His-tagged human EGFR or BCMA extracellular domain at concentrations of 3.0 or 0.4 μg/mL, respectively. After blocking with 3% (w/v) skimmed milk powder dissolved in phosphate buffered saline (PBS), antigen coated plates were used for capturing of different multivalent bispecific engagers at concentrations of 3-5 μg/mL in PBS containing 0.3% (w/v) skimmed milk powder. After washing three times with 300 μL per well of PBS containing 0.1% (v/v) Tween 20, plates were incubated with serial dilutions of biotinylated dimeric or monomeric CD16A antigen in PBS containing 0.3% (w/v) skimmed milk powder for 1.5 h at room temperature. After washing, plates were incubated with the detection conjugate Streptavidin HRP, 1:10,000 diluted in PBS containing 0.3% (w/v) skimmed milk powder for 1 h followed by washing and incubation with tetramethylbenzidine substrate (Seramun) for 1-2 min. Reaction was stopped by addition of 0.5 M H₂SO₄ (100 μL/well). Absorbance was measured at 450 nm using a multiwell plate reader, plotted and EC₅₀ values were determined by fitting a nonlinear regression model to sigmoidal dose-response curves (four parameters logistic fit) using GraphPad Prism software.

ELISA results summarized in Table 5 show overall comparable binding strengths to CD16A antigens in ELISA of bivalent and tetravalent CD16 binding engager constructs using different assay setups.

TABLE 5
Half maximal binding values for CD16 binding of tetravalent or bivalent CD16-binding
constructs targeting EGFR or BCMA analyzed in ELISA. Concentration dependent binding
of antibody constructs to coated recombinant CD16A antigen, or of soluble monomeric or
dimeric CD16 antigen variants to coated or target antigen captured antibody constructs
was analyzed in ELISA. Half maximal binding concentrations (EC₅₀) were determined
by fitting a nonlinear regression model to sigmoidal dose-response curves (four
parameters logistic fit) using GraphPad Prism software.

| | | binding of antibody | | | | binding of CD16A to | |
|---|---|---|---|---|---|---|---|
| | | constructs to | | binding of CD16A to | | target antigen- | |
| | | coated recombinant | | coated antibody | | captured antibody | |
| | | antigen | | constructs | | constructs | |
| | | CD16A | CD16A | monomeric | dimeric | monomeric | dimeric |
| | | 158F | 158V | CD16A | CD16A | CD16A | CD16A |
| | Tumor | EC₅₀ | EC₅₀ | EC₅₀ | EC₅₀ | EC₅₀ | EC₅₀ |
| Construct | target | [nM] | [nM] | [nM] | [nM] | [nM] | [nM] |
| scFv-IgAb_47 | EGFR | 0.18 | 0.16 | 2.00 | 0.89 | 1.62 | 0.76 |
| (SEQ ID NOs: | | | | | | | |
| 188 and 189) | | | | | | | |
| Bi-scDb-Fc_02 | EGFR | 0.13 | 0.11 | 3.10 | 0.97 | 1.74 | 0.79 |
| (SEQ ID NOs: | | | | | | | |
| 149) | | | | | | | |
| aBi-scDb-Fc_05 | EGFR | 0.20 | 0.16 | 4.51 | 0.99 | 5.54 | 3.00 |
| (SEQ ID NOs: | | | | | | | |
| 158 and 159) | | | | | | | |
| scFv-IgAb_18 | BCMA | 0.54 | 0.31 | 13.1 | 2.31 | 1.98 | 0.74 |
| (SEQ ID NOs: | | | | | | | |
| 172 and 173) | | | | | | | |
| Bi-scDb-Fc_01 | BCMA | 0.24 | 0.17 | 3.40 | 1.14 | 1.72 | 0.64 |
| (SEQ ID NOs: | | | | | | | |
| 148) | | | | | | | |
| aBi-scDb-Fc_02 | BCMA | 0.45 | 0.34 | 3.84 | 1.00 | 2.36 | 1.25 |
| (SEQ ID NOs: | | | | | | | |
| 152 and 153) | | | | | | | |

### Example 16: Cytotoxicity Assays on BCMA⁺ Target Cells with Bispecific NK Cell Engagers with Multivalent CD16A Binding Capacity

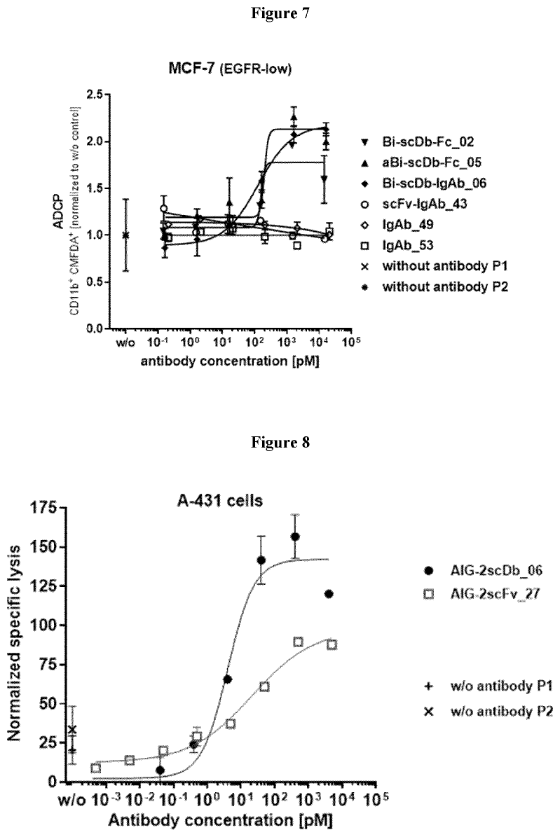

To assess potency and efficacy of BCMA-directed, multivalent NK cell engagers, and, at the same time, specificity of EGFR-targeting NK cell engagers, 4 h calcein-release cytotoxicity assays were performed on calcein-labeled BCMA⁺/EGFR⁻ MM.1S target cells with enriched NK cells as effector cells at an ET ratio of 5:1 in the presence of increasing concentrations of the indicated constructs essentially as described in Example 6. The results of the assays summarized in Table 6, and depicted in an exemplary graph in FIG. 9 clearly demonstrate potent (EC₅₀ values in the range between 1.0 pM and 2.4 pM) and efficacious (Eₘₐₓ values in the range between 46.4% and 62.8%) lysis of MM.1S target cells mediated by BCMA-specific engagers in a concentration-dependent manner. Despite multivalent binding to CD16A NK cells, EGFR-targeting engagers did not induce lysis of EGFR⁻ MM.1S target cells, demonstrating specificity of multivalent anti-CD16A innate cell engagers, that only mediate lysis of target cells by NK cells when the target antigen is expressed on the target cells.

TABLE 6
Potency and efficacy of multivalent antibody constructs determined
in 4 h cytotoxicity assays on BCMA⁺ MM.1S target cells.

| Construct | Tumor target | EC₅₀ [pM] | Eₘₐₓ [%] |
|---|---|---|---|
| Bi-scDb-Fc_01 | BCMA | 1.1 | 51.2 |
| (SEQ ID NOs: 148) | | | |
| Bi-scDb-Fc_02 | EGFR | n.a. | 0 |
| (SEQ ID NOs: 149) | | | |
| aBi-scDb-Fc_01 | BCMA | 1.0 | 46.4 |
| (SEQ ID NOs: 150 | | | |
| and 151) | | | |
| aBi-scDb-Fc_02 | BCMA | 2.3 | 62.8 |
| (SEQ ID NOs: 152 | | | |
| and 153) | | | |
| aBi-scDb-Fc_03 | BCMA | 2.4 | 51.0 |
| (SEQ ID NOs: 154 | | | |
| and 155) | | | |
| aBi-scDb-Fc_05 | EGFR | n.a. | 0 |
| (SEQ ID NOs: 156 | | | |
| and 157) | | | |

Potency (EC₅₀) and efficacy (Eₘₐₓ) were determined in 4 calcein-release cytotoxicity assays on calcein-labeled MM.1S target cells using enriched primary human NK cells as target cells and effector cells at an E:T ratio of 5:1. Mean values of three independent experiments are shown.
n.a., not applicable.

### Example 17: Efficacy of EGFR-Targeting Innate Cell Engagers in 4 h Cytotoxicity Assays on Daudi Target Cells Expressing Very Low EGFR Levels

Assessment of ADCC activity by multivalent CD16A engagement was extended to the comparison of two different CD16A-targeting sequences, using the 4 h calcein-release cytotoxicity assays against Daudi target cells as described in Example 10, using multivalent anti-CD16A constructs (Bi-scDb-Fc_02 (SEQ ID NOs: 149), scFv-Fc-scDb_04 (SEQ ID NO: 190) with constructs comprising two anti-CD16A domains (scFv-IgAb_43 (SEQ ID NOs: 174 and 175) in six independent assays using NK cells from different blood donors. The results summarized in Table 7 corroborate the superior efficacy of multivalent anti-CD16A constructs relative to bivalent anti-CD16A scFv-IgAb constructs.

TABLE 7
Efficacy of anti-EGFR antibody constructs determined
in cytotoxicity assays on Daudi target cells.

| Construct | Eₘₐₓ [%] |
|---|---|
| Bi-scDb-Fc_02 | 18.4 |
| (SEQ ID NOs: 149) | |
| scFv-Fc-scDb_04 | 22.6 |
| (SEQ ID NO: 190) | |
| scFv-IgAb_43 | 9.3 |
| (SEQ ID NOs: 174 and 175) | |

Efficacy (Eₘₐₓ) were determined in 4 calcein-release cytotoxicity assays on Daudi target cells using NK cells as effector cells at an E:T ratio of 5:1. Mean values of three independent experiments are shown.

### Example 18: 4h Phagocytosis Assay on HCT-116 Cells

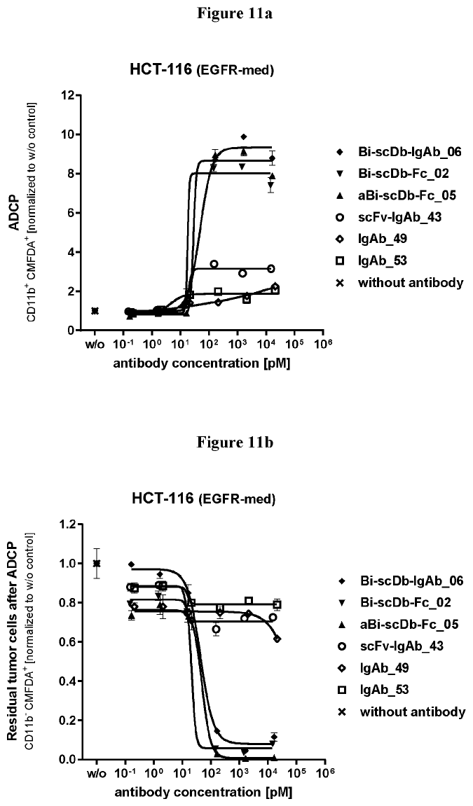

To further demonstrate the impact of multivalent CD16A engagement on the ADCP activity of EGFR-targeting innate cell engagers, 4 h phagocytosis assays were performed on HCT-116 target cells expressing medium levels of EGFR (mean SABC: 33,822). As described in Example 8, the multivalent anti-CD16A constructs (Bi-scDb-Fc_02 (SEQ ID NOs: 149), aBi-scDb-Fc_05 (SEQ ID NOs: 158 and 159), Bi-scDb-IgAb_06 (SEQ ID NOs: 162 and 163)) were compared with constructs comprising two anti-CD16A domains (scFv-IgAb_43 (SEQ ID NOs: 174 and 175)), with Fc-wildtype anti-EGFR IgG1 (IgAb_49 (SEQ ID NOs: 164 and 165)) and with Fc-enhanced (S239D/1332E) anti-EGFR IgG1 (IgAb_53 (SEQ ID NOs: 168 and 169)) in three independent assays using macrophages generated from monocytes of different blood donors. A representative exemplary graph in FIG. 11A demonstrates phagocytosis induction of multivalent anti-CD16A constructs. In contrast, scFv-IgAb_43, IgAb_49 and IgAb_53 showed only low level of phagocytosis of HCT-116 target cells by macrophages. In addition, a representative exemplary graph in FIG. 11B shows the concentration-dependent loss of HCT-116 target cells mediated by multivalent anti-CD16A constructs as measured at the end of the 4 h co-culture with macrophages. In contrast, did not show a concentration-dependent loss of HCT-116 target cells in the presence of scFv-IgAb_43, IgAb_49 and IgAb_53. These results clearly show the advantage of multivalent anti-CD16A engagement for ADCP-mediating innate cell engagers.

### Example 19: Efficacy of CD19-Targeting Innate Cell Engagers in 4 h Cytotoxicity Assays on Daudi Target Cells

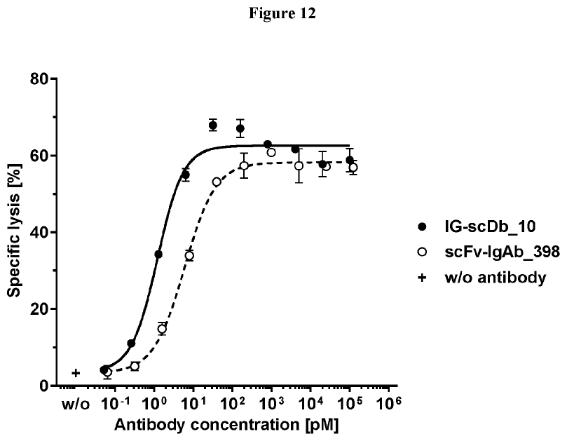

To assess the impact of multivalent CD16A engagement on the ADCC activity of CD19 targeting innate cell engagers, 4 h calcein-release cytotoxicity assays were performed on Daudi target cells expressing high levels of CD19 and enriched primary human NK cells as effector cells at an ET ratio of 5:1. The assays performed as described in Example 6 were used to compare a multivalent anti-CD16A construct (IG-scDb_10 (SEQ ID NOs: 191 and 192)) with a construct comprising two anti-CD16A domains (scFv-IgAb_398 (SEQ ID NOs: 193 and 194)) in two independent assays using NK cells from different blood donors. A representative exemplary graph in FIG. 12 demonstrates superior potency and efficacy of the multivalent anti-CD16A construct relative to the bivalent anti-CD16A scFv-IgAb construct. This result clearly shows the advantage of multivalent anti-CD16A engagement for ADCC-mediating innate cell engagers, and bivalent tumor-targeting.

Sequence Listing

| SEQ | | Sequence |
|---|---|---|
| ID | | |
| NO | Description | |
| 1 | Linker: | GGGS |
| 2 | Linker: | GGSGGS |
| 3 | Linker: | GGSGGSGGS |
| 4 | Linker: | GGSGGSGGSGGSGGSGGS |
| 5 | Linker: | GGSGGSGGSGGSGGSGGSGGS |
| 6 | Linker: | GGGGS |
| 7 | Linker: | GGGGSGGGGS |
| 8 | Linker: | GGGGSGGGGSGGGGSGGGGS |
| 9 | Linker: | GGGGGGGGSGGGGSGGGGSGGGGSGGG |
| | | GS |
| 10 | hinge: | EPKSCDKTHTCPPCP |
| 11 | upper.hinge: | EPKSCDKTHT |
| 12 | middle.hinge: | DKTHTCPPCP |
| 13 | human CD16A: | MWQLLLPTALLLLVSAGMRTEDLPKAV |
| | | VFLEPQWYRVLEKDSVTLKCQGAYSPE |
| | | DNSTQWFHNESLISSQASSYFIDAATV |
| | | DDSGEYRCQTNLSTLSDPVQLEVHIGW |
| | | LLLQAPRWVFKEEDPIHLRCHSWKNTA |
| | | LHKVTYLQNGKGRKYFHHNSDFYIPKA |
| | | TLKDSGSYFCRGLFGSKNVSSETVNIT |
| | | ITQGLAVSTISSFFPPGYQVSFCLVMV |
| | | LLFAVDTGLYFSVKTNIRSSTRDWKDH |
| | | KFKWRKDPQDK |
| 14 | cynomolgus | MWQLLLPTALLLLVSAGMRAEDLPKAV |
| | CD16: | VFLEPQWYRVLEKDRVTLKCQGAYSPE |
| | | DNSTRWFHNESLISSQTSSYFIAAARV |
| | | NNSGEYRCQTSLSTLSDPVQLEVHIGW |
| | | LLLQAPRWVFKEEESIHLRCHSWKNTL |
| | | LHKVTYLQNGKGRKYFHQNSDFYIPKA |
| | | TLKDSGSYFCRGLIGSKNVSSETVNIT |
| | | ITQDLAVSSISSFFPPGYQVSFCLVMV |
| | | LLFAVDTGLYFSMKKSIPSSTRDWEDH |
| | | KFKWSKDPQDK |
| 15 | human CD16B: | MWQLLLPTALLLLVSAGMRTEDLPKAV |
| | | VFLEPQWYSVLEKDSVTLKCQGAYSPE |
| | | DNSTQWFHNESLISSQASSYFIDAATV |
| | | NDSGEYRCQTNLSTLSDPVQLEVHIGW |
| | | LLLQAPRWVFKEEDPIHLRCHSWKNTA |
| | | LHKVTYLQNGKDRKYFHHNSDFHIPKA |
| | | TLKDSGSYFCRGLVGSKNVSSETVNIT |
| | | ITQGLAVSTISSESPPGYQVSFCLVMV |
| | | LLFAVDTGLYFSVKTNI |
| 16 | human NKG2D: | MGWIRGRRSRHSWEMSEFHNYNLDLKK |
| | | SDFSTRWQKQRCPVVKSKCRENASPFF |
| | | FCCFIAVAMGIRFIIMVAIWSAVELNS |
| | | LFNQEVQIPLTESYCGPCPKNWICYKN |
| | | NCYQFFDESKNWYESQASCMSQNASLL |
| | | KVYSKEDQDLLKLVKSYHWMGLVHIPT |
| | | NGSWQWEDGSILSPNLLTIIEMQKGDC |
| | | ALYASSFKGYIENCSTPNTYICMQRTV |
| 17 | human NKp30: | MAWMLLLILIMVHPGSCALWVSQPPEI |
| | | RTLEGSSAFLPCSFNASQGRLAIGSVT |
| | | WERDEVVPGKEVRNGTPEFRGRLAPLA |
| | | SSRFLHDHQAELHIRDVRGHDASIYVC |
| | | RVEVLGLGVGTGNGTRLVVEKEHPQLG |
| | | AGTVLLLRAGFYAVSFLSVAVGSTVYY |
| | | QGKCLTWKGPRRQLPAVVPAPLPPPCG |
| | | SSAHLLPPVPGG |
| 18 | human NKp44: | MAWRALHPLLLLLLLFPGSQAQSKAQV |
| | | LQSVAGQTLTVRCQYPPTGSLYEKKGW |
| | | CKEASALVCIRLVTSSKPRTMAWTSRF |
| | | TIWDDPDAGFFTVTMTDLREEDSGHYW |
| | | CRIYRPSDNSVSKSVRFYLVVSPASAS |
| | | TQTSWTPRDLVSSQTQTQSCVPPTAGA |
| | | RQAPESPSTIPVPSQPQNSTLRPGPAA |
| | | PIALVPVFCGLLVAKSLVLSALLVWWG |
| | | DIWWKTMMELRSLDTQKATCHLQQVTD |
| | | LPWTSVSSPVEREILYHTVARTKISDD |
| | | DDEHTL |
| 19 | human NKp46: | MSSTLPALLCVGLCLSQRISAQQQTLP |
| | | KPFIWAEPHEMVPKEKQVTICCQGNYG |
| | | AVEYQLHFEGSLFAVDRPKPPERINKV |
| | | KFYIPDMNSRMAGQYSCIYRVGELWSE |
| | | PSNLLDLVVTEMYDTPTLSVHPGPEVI |
| | | SGEKVTFYCRLDTATSMFLLLKEGRSS |
| | | HVQRGYGKVQAEFPLGPVTTAHRGTYR |
| | | CFGSYNNHAWSFPSEPVKLLVTGDIEN |
| | | TSLAPEDPTFPADTWGTYLLTTETGLQ |
| | | KDHALWDHTAQNLLRMGLAFLVLVALV |
| | | WFLVEDWLSRKRTRERASRASTWEGRR |
| | | RLNTQTL |
| 20 | human SLAMF7: | MAGSPTCLTLIYILWQLTGSAASGPVK |
| | | ELVGSVGGAVTFPLKSKVKQVDSIVWT |
| | | ENTTPLVTIQPEGGTIIVTQNRNRERV |
| | | DFPDGGYSLKLSKLKKNDSGIYYVGIY |
| | | SSSLQQPSTQEYVLHVYEHLSKPKVTM |
| | | GLQSNKNGTCVTNLTCCMEHGEEDVIY |
| | | TWKALGQAANESHNGSILPISWRWGES |
| | | DMTFICVARNPVSRNESSPILARKLCE |
| | | GAADDPDSSMVLLCLLLVPLLLSLFVL |
| | | GLFLWELKRERQEEYIEEKKRVDICRE |
| | | TPNICPHSGENTEYDTIPHTNRTILKE |
| | | DPANTVYSTVEIPKKMENPHSLLTMPD |
| | | TPRLFAYENVI |
| 21 | human 2B4: | MLGQVVTLILLLLLKVYQGKGCQGSAD |
| | | HVVSISGVPLQLQPNSIQTKVDSIAWK |
| | | KLLPSQNGFHHILKWENGSLPSNTSND |
| | | RFSFIVKNLSLLIKAAQQQDSGLYCLE |
| | | VTSISGKVQTATFQVFVFESLLPDKVE |
| | | KPRLQGQGKILDRGRCQVALSCLVSRD |
| | | GNVSYAWYRGSKLIQTAGNLTYLDEEV |
| | | DINGTHTYTCNVSNPVSWESHTLNLTQ |
| | | DCQNAHQEFREWPFLVIIVILSALFLG |
| | | TLACFCVWRRKRKEKQSETSPKEFLTI |
| | | YEDVKDLKTRRNHEQEQTFPGGGSTIY |
| | | SMIQSQSSAPTSQEPAYTLYSLIQPSR |
| | | KSGSRKRNHSPSENSTIYEVIGKSQPK |
| | | AQNPARLSRKELENFDVYS |
| 22 | human OX40: | MCVGARRLGRGPCAALLLLGLGLSTVT |
| | | GLHCVGDTYPSNDRCCHECRPGNGMVS |
| | | RCSRSQNTVCRPCGPGFYNDVVSSKPC |
| | | KPCTWCNLRSGSERKQLCTATQDTVCR |
| | | CRAGTQPLDSYKPGVDCAPCPPGHFSP |
| | | GDNQACKPWTNCTLAGKHTLQPASNSS |
| | | DAICEDRDPPATQPQETQGPPARPITV |
| | | QPTEAWPRTSQGPSTRPVEVPGGRAVA |
| | | AILGLGLVLGLLGPLAILLALYLLRRD |
| | | QRLPPDAHKPPGGGSFRTPIQEEQADA |
| | | HSTLAKI |
| 23 | human CD137: | MGNSCYNIVATLLLVLNFERTRSLQDP |
| | | CSNCPAGTFCDNNRNQICSPCPPNSFS |
| | | SAGGQRTCDICRQCKGVFRTRKECSST |
| | | SNAECDCTPGFHCLGAGCSMCEQDCKQ |
| | | GQELTKKGCKDCCFGTFNDQKRGICRP |
| | | WTNCSLDGKSVLVNGTKERDVVCGPSP |
| | | ADLSPGASSVTPPAPAREPGHSPQIIS |
| | | FFLALTSTALLFLLFFLTLRESVVKRG |
| | | RKKLLYIFKQPFMRPVQTTQEEDGCSC |
| | | RFPEEEEGGCEL |
| 24 | human CD89: | MDPKQTTLLCLVLCLGQRIQAQEGDFP |
| | | MPFISAKSSPVIPLDGSVKIQCQAIRE |
| | | AYLTQLMIIKNSTYREIGRRLKFWNET |
| | | DPEFVIDHMDANKAGRYQCQYRIGHYR |
| | | FRYSDTLELVVTGLYGKPFLSADRGLV |
| | | LMPGENISLTCSSAHIPFDRFSLAKEG |
| | | ELSLPQHQSGEHPANFSLGPVDLNVSG |
| | | IYRCYGWYNRSPYLWSFPSNALELVVT |
| | | DSIHQDYTTQNLIRMAVAGLVLVALLA |
| | | ILVENWHSHTALNKEASADVAEPSWSQ |
| | | QMCQPGLTFARTPSVCK |
| 25 | human CD160: | MLLEPGRGCCALAILLAIVDIQSGGCI |
| | | NITSSASQEGTRLNLICTVWHKKEEAE |
| | | GFVVFLCKDRSGDCSPETSLKQLRLKR |
| | | DPGIDGVGEISSQLMFTISQVTPLHSG |
| | | TYQCCARSQKSGIRLQGHFFSILFTET |
| | | GNYTVTGLKQRQHLEFSHNEGTLSSGF |
| | | LQEKVWVMLVTSLVALQAL |
| 26 | hinge: | EPKSCDKTHTCPPCP |
| 27 | upper.hinge: | EPKSCDKTHT |
| 28 | middle.hinge: | DKTHTCPPCP |
| 29 | IgG2 subtype | ERKCCVECPPCP |
| | hinge: | |
| 30 | IgG3 subtype | ELKTPLDTTHTCPRCP |
| | hinge | |
| 31 | IgG3 subtype | ELKTPLGDTTHTCPRCP |
| | hinge | |
| 32 | IgG4 subtype | ESKYGPPCPSCP |
| | hinge | |
| 33 | Human IgG1 CH1, | ASTKGPSVFPLAPSSKSTSGGTAALGC |
| | CH2 and CH3 | LVKDYFPEPVTVSWNSGALTSGVHTFP |
| | heavy chain | AVLQSSGLYSLSSVVTVPSSSLGTQTY |
| | constant | ICNVNHKPSNTKVDKKVEPKSCDKTHT |
| | domain: | CPPCPAPELLGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 34 | Human IgG1 CH1, | ASTKGPSVFPLAPSSKSTSGGTAALGC |
| | CH2 and CH3 | LVKDYFPEPVTVSWNSGALTSGVHTFP |
| | heavy chain | AVLQSSGLYSLSSVVTVPSSSLGTQTY |
| | constant domain | ICNVNHKPSNTKVDKKVEPKSCDKTHT |
| | with silencing | CPPCPAPEFEGGPSVFLFPPKPKDTLM |
| | mutation-1: | ISRTPEVTCVVVAVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 35 | Human IgG1 CH1, | ASTKGPSVFPLAPSSKSTSGGTAALGC |
| | CH2 and CH3 | LVKDYFPEPVTVSWNSGALTSGVHTFP |
| | heavy chain | AVLQSSGLYSLSSVVTVPSSSLGTQTY |
| | constant domain | ICNVNHKPSNTKVDKKVEPKSCDKTHT |
| | with enhancing | CPPCPAPELLGGPDVFLFPPKPKDTLM |
| | mutation-1: | ISRTPEVTCVVVDVSHEDPEVKFNWYV |
| | | DGVEVHNAKTKPREEQYNSTYRVVSVL |
| | | TVLHQDWLNGKEYKCKVSNKALPAPEE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPG |
| 36 | Human lambda | GQPKAAPSVTLEPPSSEELQANKATLV |
| | light chain | CLISDFYPGAVTVAWKADSSPVKAGVE |
| | constant | TTTPSKQSNNKYAASSYLSLTPEQWKS |
| | domain: | HRSYSCQVTHEGSTVEKTVAPTECS |
| 37 | Human Kappa | RTVAAPSVFIFPPSDEQLKSGTASVVC |
| | light chain | LLNNFYPREAKVQWKVDNALQSGNSQE |
| | constant | SVTEQDSKDSTYSLSSTLTLSKADYEK |
| | domain: | HKVYACEVTHQGLSSPVTKSENRGEC |
| 38 | CH1 heavy chain | ASTKGPSVFPLAPSSKSTSGGTAALGC |
| | constant | LVKDYFPEPVTVSWNSGALTSGVHTFP |
| | domain: | AVLQSSGLYSLSSVVTVPSSSLGTQTY |
| | | ICNVNHKPSNTKVDKKV |
| 39 | CH2-CH3 heavy | APELLGGPSVFLFPPKPKDTLMISRTP |
| | chain constant | EVTCVVVDVSHEDPEVKENWYVDGVEV |
| | domain: | HNAKTKPREEQYNSTYRVVSVLTVLHQ |
| | | DWLNGKEYKCKVSNKALPAPIEKTISK |
| | | AKGQPREPQVYTLPPSREEMTKNQVSL |
| | | TCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 40 | CH2-CH3 heavy | APEFEGGPSVFLFPPKPKDTLMISRTP |
| | chain constant | EVTCVVVAVSHEDPEVKENWYVDGVEV |
| | domain with | HNAKTKPREEQYNSTYRVVSVLTVLHQ |
| | silencing | DWLNGKEYKCKVSNKALPAPIEKTISK |
| | mutation-1: | AKGQPREPQVYTLPPSREEMTKNQVSL |
| | | TCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 41 | CH2-CH3 heavy | APEFEGGPSVFLFPPKPKDTLMISRTP |
| | chain constant | EVTCVVVAVSHEDPEVKENWYVDGVEV |
| | domain with | HNAKTKPREEQYGSTYRVVSVLTVLHQ |
| | silencing | DWLNGKEYKCKVSNKALPAPIEKTISK |
| | mutation-2: | AKGQPREPQVYTLPPSREEMTKNQVSL |
| | | TCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 42 | CH2-CH3 heavy | APELLGGPDVFLFPPKPKDTLMISRTP |
| | chain constant | EVTCVVVDVSHEDPEVKFNWYVDGVEV |
| | domain with | HNAKTKPREEQYNSTYRVVSVLTVLHQ |
| | enhancing | DWLNGKEYKCKVSNKALPAPEEKTISK |
| | mutation-1: | AKGQPREPQVYTLPPSREEMTKNQVSL |
| | | TCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 43 | Hole chain CH2- | APELLGGPSVFLFPPKPKDTLMISRTP |
| | CH3 heavy chain | EVTCVVVDVSHEDPEVKENWYVDGVEV |
| | constant | HNAKTKPREEQYNSTYRVVSVLTVLHQ |
| | domain-1: | DWLNGKEYKCKVSNKALPAPIEKTISK |
| | | AKGQPREPQVYTLPPSREEMTKNQVSL |
| | | TCLVKGFYPSDIAVEWESNGQPENNYK |
| | | TTPPVLDSDGSFFLTSKLTVDKSRWQQ |
| | | GNVFSCSVMHEALHNHYTQKSLSLSPG |
| 44 | Knob chain CH2- | APELLGGPSVFLFPPKPKDTLMISRTP |
| | CH3 heavy chain | EVTCVVVDVSHEDPEVKENWYVDGVEV |
| | constant | HNAKTKPREEQYNSTYRVVSVLTVLHQ |
| | domain-1: | DWLNGKEYKCKVSNKALPAPIEKTISK |
| | | AKGQPREPQVYTLPPSREEMTKNQVSL |
| | | YCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 45 | Hole chain CH2- | APELLGGPSVFLFPPKPKDTLMISRTP |
| | CH3 heavy chain | EVTCVVVDVSHEDPEVKFNWYVDGVEV |
| | constant | HNAKTKPREEQYNSTYRVVSVLTVLHQ |
| | domain-2: | DWLNGKEYKCKVSNKALPAPIEKTISK |
| | | AKGQPREPQVYTLPPSREEMTKNQVSL |
| | | SCAVKGFYPSDIAVEWESNGQPENNYK |
| | | TTPPVLDSDGSFFLVSKLTVDKSRWQQ |
| | | GNVFSCSVMHEALHNHYTQKSLSLSPG |
| 46 | Knob chain CH2- | APELLGGPSVFLFPPKPKDTLMISRTP |
| | CH3 heavy chain | EVTCVVVDVSHEDPEVKFNWYVDGVEV |
| | constant | HNAKTKPREEQYNSTYRVVSVLTVLHQ |
| | domain-2: | DWLNGKEYKCKVSNKALPAPIEKTISK |
| | | AKGQPREPQVYTLPPSREEMTKNQVSL |
| | | WCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 47 | Hole chain CH2- | APEFEGGPSVFLFPPKPKDTLMISRTP |
| | CH3 heavy chain | EVTCVVVAVSHEDPEVKFNWYVDGVEV |
| | constant | HNAKTKPREEQYNSTYRVVSVLTVLHQ |
| | domain-1 with | DWLNGKEYKCKVSNKALPAPIEKTISK |
| | silencing | AKGQPREPQVYTLPPSREEMTKNQVSL |
| | mutation-1: | TCLVKGFYPSDIAVEWESNGQPENNYK |
| | | TTPPVLDSDGSFFLTSKLTVDKSRWQQ |
| | | GNVFSCSVMHEALHNHYTQKSLSLSPG |
| 48 | Knob chain CH2- | APEFEGGPSVFLFPPKPKDTLMISRTP |
| | CH3 heavy chain | EVTCVVVAVSHEDPEVKFNWYVDGVEV |
| | constant | HNAKTKPREEQYNSTYRVVSVLTVLHQ |
| | domain-1 with | DWLNGKEYKCKVSNKALPAPIEKTISK |
| | silencing | AKGQPREPQVYTLPPSREEMTKNQVSL |
| | mutation-1: | YCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 49 | Hole chain CH2- | APEFEGGPSVFLFPPKPKDTLMISRTP |
| | CH3 heavy chain | EVTCVVVAVSHEDPEVKFNWYVDGVEV |
| | constant | HNAKTKPREEQYNSTYRVVSVLTVLHQ |
| | domain-2 with | DWLNGKEYKCKVSNKALPAPIEKTISK |
| | silencing | AKGQPREPQVYTLPPSREEMTKNQVSL |
| | mutation-1: | SCAVKGFYPSDIAVEWESNGQPENNYK |
| | | TTPPVLDSDGSFFLVSKLTVDKSRWQQ |
| | | GNVFSCSVMHEALHNHYTQKSLSLSPG |
| 50 | Knob chain CH2- | APEFEGGPSVFLFPPKPKDTLMISRTP |
| | CH3 heavy chain | EVTCVVVAVSHEDPEVKENWYVDGVEV |
| | constant | HNAKTKPREEQYNSTYRVVSVLTVLHQ |
| | domain-2 with | DWLNGKEYKCKVSNKALPAPIEKTISK |
| | silencing | AKGQPREPQVYTLPPSREEMTKNQVSL |
| | mutation-1: | WCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 51 | Hole chain CH2- | APEFEGGPSVFLFPPKPKDTLMISRTP |
| | CH3 heavy chain | EVTCVVVAVSHEDPEVKENWYVDGVEV |
| | constant | HNAKTKPREEQYGSTYRVVSVLTVLHQ |
| | domain-1 with | DWLNGKEYKCKVSNKALPAPIEKTISK |
| | silencing | AKGQPREPQVYTLPPSREEMTKNQVSL |
| | mutation-2: | TCLVKGFYPSDIAVEWESNGQPENNYK |
| | | TTPPVLDSDGSFFLTSKLTVDKSRWQQ |
| | | GNVFSCSVMHEALHNHYTQKSLSLSPG |
| 52 | Knob chain CH2- | APEFEGGPSVFLFPPKPKDTLMISRTP |
| | CH3 heavy chain | EVTCVVVAVSHEDPEVKENWYVDGVEV |
| | constant-1 | HNAKTKPREEQYGSTYRVVSVLTVLHQ |
| | domain with | DWLNGKEYKCKVSNKALPAPIEKTISK |
| | silencing | AKGQPREPQVYTLPPSREEMTKNQVSL |
| | mutation-2: | YCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 53 | Hole chain CH2- | APEFEGGPSVFLFPPKPKDTLMISRTP |
| | CH3 heavy chain | EVTCVVVAVSHEDPEVKFNWYVDGVEV |
| | constant | HNAKTKPREEQYGSTYRVVSVLTVLHQ |
| | domain-2 with | DWLNGKEYKCKVSNKALPAPIEKTISK |
| | silencing | AKGQPREPQVYTLPPSREEMTKNQVSL |
| | mutation-2: | SCAVKGFYPSDIAVEWESNGQPENNYK |
| | | TTPPVLDSDGSFFLVSKLTVDKSRWQQ |
| | | GNVFSCSVMHEALHNHYTQKSLSLSPG |
| 54 | Knob chain CH2- | APEFEGGPSVFLFPPKPKDTLMISRTP |
| | CH3 heavy chain | EVTCVVVAVSHEDPEVKENWYVDGVEV |
| | constant | HNAKTKPREEQYGSTYRVVSVLTVLHQ |
| | domain-2 with | DWLNGKEYKCKVSNKALPAPIEKTISK |
| | silencing | AKGQPREPQVYTLPPSREEMTKNQVSL |
| | mutation-2: | WCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 55 | Hole chain CH2- | APELLGGPDVFLFPPKPKDTLMISRTP |
| | CH3 heavy chain | EVTCVVVDVSHEDPEVKENWYVDGVEV |
| | constant | HNAKTKPREEQYNSTYRVVSVLTVLHQ |
| | domain-1 with | DWLNGKEYKCKVSNKALPAPEEKTISK |
| | enhancing | AKGQPREPQVYTLPPSREEMTKNQVSL |
| | mutation-1: | TCLVKGFYPSDIAVEWESNGQPENNYK |
| | | TTPPVLDSDGSFFLTSKLTVDKSRWQQ |
| | | GNVFSCSVMHEALHNHYTQKSLSLSPG |
| 56 | Knob chain CH2- | APELLGGPDVFLFPPKPKDTLMISRTP |
| | CH3 heavy chain | EVTCVVVDVSHEDPEVKENWYVDGVEV |
| | constant | HNAKTKPREEQYNSTYRVVSVLTVLHQ |
| | domain-1 with | DWLNGKEYKCKVSNKALPAPEEKTISK |
| | enhancing | AKGQPREPQVYTLPPSREEMTKNQVSL |
| | mutation-1: | YCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 57 | Hole chain CH2- | APELLGGPDVFLFPPKPKDTLMISRTP |
| | CH3 heavy chain | EVTCVVVDVSHEDPEVKFNWYVDGVEV |
| | constant | HNAKTKPREEQYNSTYRVVSVLTVLHQ |
| | domain-2 with | DWLNGKEYKCKVSNKALPAPEEKTISK |
| | enhancing | AKGQPREPQVYTLPPSREEMTKNQVSL |
| | mutation-1: | SCAVKGFYPSDIAVEWESNGQPENNYK |
| | | TTPPVLDSDGSFFLVSKLTVDKSRWQQ |
| | | GNVFSCSVMHEALHNHYTQKSLSLSPG |
| 58 | Knob chain CH2- | APELLGGPDVFLFPPKPKDTLMISRTP |
| | CH3 heavy chain | EVTCVVVDVSHEDPEVKFNWYVDGVEV |
| | constant | HNAKTKPREEQYNSTYRVVSVLTVLHQ |
| | domain-2 with | DWLNGKEYKCKVSNKALPAPEEKTISK |
| | enhancing | AKGQPREPQVYTLPPSREEMTKNQVSL |
| | mutation-1: | WCLVKGFYPSDIAVEWESNGQPENNYK |
| | | TTPPVLDSDGSFFLYSKLTVDKSRWQQ |
| | | GNVFSCSVMHEALHNHYTQKSLSLSPG |
| 59 | VH CD16A-1: | QVQLVQSGAEVKKPGESLKVSCKASGY |
| | | TFTSYYMHWVRQAPGQGLEWMGAIEPM |
| | | YGSTSYAQKFQGRVTMTRDTSTSTVYM |
| | | ELSSLRSEDTAVYYCARGSAYYYDFAD |
| | | YWGQGTLVTVSS |
| 60 | VH CD16A-2: | QVQLVQSGAEVKKPGESLKVSCKASGY |
| | | TFTNYYMQWVRQAPGQGLEWMGIINPS |
| | | GGVTSYAQKFQGRVTMTRDTSTSTVYM |
| | | ELSSLRSEDTAVYYCARGSAYYYDFAD |
| | | YWGQGTLVTVSS |
| 61 | VH CD16A-3: | QVQLVQSGAEVKKPGESLKVSCKASGY |
| | | TFTSYYMHWVRQAPGQGLEWMGIINPS |
| | | GGSTSYAQKFQGRVTMTRDTSTSTVYM |
| | | ELSSLRSEDTAVYYCARGSAYYYDFAD |
| | | YWGQGTLVTVSS |
| 62 | VH NKp46: | QVQLQQSGPELVKPGASVKMSCKASGY |
| | | TFTDYVINWGKQRSGQGLEWIGEIYPG |
| | | SGTNYYNEKFKAKATLTADKSSNIAYM |
| | | QLSSLTSEDSAVYFCARRGRYGLYAMD |
| | | YWGQGTSVTVSS |
| 63 | VH NKG2D: | QVQLVESGGGLVKPGGSLRLSCAASGF |
| | | TFSSYGMHWVRQAPGKGLEWVAFIRYD |
| | | GSNKYYADSVKGRFTISRDNSKNTLYL |
| | | QMNSLRAEDTAVYYCAKDRGLGDGTYF |
| | | DYWGQGTTVTVSS |
| 64 | VH BCMA: | EVQLLESGGGLVQPGGSLRLSCAASGF |
| | | TFSNYDMAWVRQAPGKGLEWVSSISTR |
| | | GDITSYRDSVKGRFTISRDNSKNTLYL |
| | | QMNSLRAEDTAVYYCARQDYYTDYMGF |
| | | AYWGQGTLVTVSS |
| 65 | VH CD19 | EVQLVESGGGLVKPGGSLKLSCAASGY |
| | (MOR208): | TFTSYVMHWVRQAPGKGLEWIGYINPY |
| | | NDGTKYNEKFQGRVTISSDKSISTAYM |
| | | ELSSLRSEDTAMYYCARGTYYYGTRVF |
| | | DYWGQGTLVTVSS |
| 66 | VH EGFR: | QVQLQESGPGLVKPSETLSLTCTVSGG |
| | | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSS |
| 67 | VH HER2 (4D5): | EVQLVESGGGLVQPGGSLRLSCAASGF |
| | | NIKDTYIHWVRQAPGKGLEWVARIYPT |
| | | NGYTRYADSVKGRFTISADTSKNTAYL |
| | | QMNSLRAEDTAVYYCSRWGGDGFYAMD |
| | | YWGQGTLVTVSS |
| 68 | VL CD16A-1: | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVL |
| 69 | VL CD16A-2: | SYVLTQPSSVSVAPGQTARITCGGNNI |
| | | GSKSVHWYQQKPGQAPVLVIYQDKKRP |
| | | SGIPERFSGSNSGNTATLTISGTQAMD |
| | | EADYYCQVWDDYIVLFGGGTKLTVL |
| 70 | VL CD16A-3: | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVL |
| 71 | VL NKp46: | DIQMTQTTSSLSASLGDRVTISCRASQ |
| | | DISNYLNWYQQKPDGTVKLLIYYTSRL |
| | | HSGVPSRFSGSGSGTDYSLTINNLEQE |
| | | DIATYFCQQGNTRPWTFGGGTKLEIK |
| 72 | VL NKG2D: | QSALTQPASVSGSPGQSITISCSGSSS |
| | | NIGNNAVNWYQQLPGKAPKLLIYYDDL |
| | | LPSGVSDRFSGSKSGTSAFLAISGLQS |
| | | EDEADYYCAAWDDSLNGPVFGGGTKLT |
| | | VL |
| 73 | VL BCMA: | AIQMTQSPSSLSASVGDRVTITCRASE |
| | | DIYNGLAWYQQKPGKAPKLLIYGASSL |
| | | QDGVPSRFSGSGSGTEFTLTISSLQPE |
| | | DEATYYCAGPHKYPLTFGGGTKVEIK |
| 74 | VL CD19 | DIVMTQSPATLSLSPGERATLSCRSSK |
| | (MOR208): | SLQNVNGNTYLYWFQQKPGQSPQLLIY |
| | | RMSNLNSGVPDRFSGSGSGTEFTLTIS |
| | | SLEPEDFAVYYCMQHLEYPITFGAGTK |
| | | LEIK |
| 75 | VL EGFR: | QPVLTQPPSVSVAPGKTARITCGGNNI |
| | | GSKSVHWYQQKPGQAPVLVIYYDSDRP |
| | | SGIPERFSGSNSGNTATLTISRVEAGD |
| | | EADYYCQVWDTSSDHVLFGGGTKLTVL |
| 76 | VL HER2 (4D5): | DIQMTQSPSSLSASVGDRVTITCRASQ |
| | | DVNTAVAWYQQKPGKAPKLLIYSASFL |
| | | YSGVPSRESGSRSGTDFTLTISSLQPE |
| | | DFATYYCQQHYTTPPTFGQGTKVEIK |
| 77 | HCDR1 CD16A-1/- | SYYMH |
| | 3: | |
| 78 | HCDR2 CD16A-1: | AIEPMYGSTSYAQKFQG |
| 79 | HCDR3 CD16A-1/- | GSAYYYDFADY |
| | 3: | |
| 80 | LCDR1 CD16A-1/- | GGHNIGSKNVH |
| | 3: | |
| 81 | LCDR2 CD16A-1/- | QDNKRPS |
| | 3: | |
| 82 | LCDR3 CD16A-1/- | QVWDNYSVL |
| | 3: | |
| 83 | HCDR1 CD16A-2: | NYYMQ |
| 84 | HCDR2 CD16A-2: | IINPSGGVTSYAQKFQG |
| 85 | HCDR3 CD16A-2: | GSAYYYDFADY |
| 86 | LCDR1 CD16A-2: | GGNNIGSKSVH |
| 87 | LCDR2 CD16A-2: | QDKKRPS |
| 88 | LCDR3 CD16A-2: | QVWDDYIVL |
| 89 | HCDR2 CD16A-3: | IINPSGGSTSYAQKFQG |
| 90 | HCDR1 Nkp46: | DYVIN |
| 91 | HCDR2 Nkp46: | EIYPGSGTNYYNEKFKA |
| 92 | HCDR3 Nkp46: | RGRYGLYAMDY |
| 93 | LCDR1 Nkp46: | RASQDISNYLN |
| 94 | LCDR2 Nkp46: | YTSRLHS |
| 95 | LCDR3 Nkp46: | QQGNTRPWT |
| 96 | HCDR1 NKG2D: | SYGMH |
| 97 | HCDR2 NKG2D: | FIRYDGSNKYYADSVKG |
| 98 | HCDR3 NKG2D: | DRGLGDGTYFDY |
| 99 | LCDR1 NKG2D: | SGSSSNIGNNAVN |
| 100 | LCDR2 NKG2D: | YDDLLPS |
| 101 | LCDR3 NKG2D: | AAWDDSLNGPV |
| 102 | HCDR1 BCMA: | NYDMA |
| 103 | HCDR2 BCMA: | SISTRGDITSYRDSVKG |
| 104 | HCDR3 BCMA: | QDYYTDYMGFAY |
| 105 | LCDR1 BCMA: | RASEDIYNGLA |
| 106 | LCDR2 BCMA: | GASSLQD |
| 107 | LCDR3 BCMA: | AGPHKYPLT |
| 108 | HCDR1 CD19 | SYVMH |
| | (MOR208): | |
| 109 | HCDR2 CD19 | YINPYNDGTKYNEKFQG |
| | (MOR208): | |
| 110 | HCDR3 CD19 | GTYYYGTRVFDY |
| | (MOR208): | |
| 111 | LCDR1 CD19 | RSSKSLQNVNGNTYLY |
| | (MOR208): | |
| 112 | LCDR2 CD19 | RMSNLNS |
| | (MOR208): | |
| 113 | LCDR3 CD19 | MQHLEYPIT |
| | (MOR208): | |
| 114 | HCDR1 EGFR: | SGSYYWS |
| 115 | HCDR2 EGFR: | YIYYSGSTNYNPSLKS |
| 116 | HCDR3 EGFR: | NPISIPAFDI |
| 117 | LCDR1 EGFR: | GGNNIGSKSVH |
| 118 | LCDR2 EGFR: | YDSDRPS |
| 119 | LCDR3 EGFR: | QVWDTSSDHVL |
| 120 | HCDR1 HER2 | DTYIH |
| | (4D5): | |
| 121 | HCDR2 HER2 | RIYPTNGYTRYADSVKG |
| | (4D5): | |
| 122 | HCDR3 HER2 | WGGDGFYAMDY |
| | (4D5): | |
| 123 | LCDR1 HER2 | RASQDVNTAVA |
| | (4D5): | |
| 124 | LCDR2 HER2 | SASFLYS |
| | (4D5): | |
| 125 | LCDR3 HER2 | QQHYTTPPT |
| | (4D5): | |
| 126 | scDb_CD16A-1: | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLFGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSS |
| 127 | scDb_CD16A-2: | SYVLTQPSSVSVAPGQTARITCGGNNI |
| | | GSKSVHWYQQKPGQAPVLVIYQDKKRP |
| | | SGIPERFSGSNSGNTATLTISGTQAMD |
| | | EADYYCQVWDDYIVLFGGGTKLTVLGG |
| | | SGGSQVQLVQSGAEVKKPGESLKVSCK |
| | | ASGYTFTNYYMQWVRQAPGQGLEWMGI |
| | | INPSGGVTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTARI |
| | | TCGGNNIGSKSVHWYQQKPGQAPVLVI |
| | | YQDKKRPSGIPERFSGSNSGNTATLTI |
| | | SGTQAMDEADYYCQVWDDYIVLFGGGT |
| | | KLTVLGGSGGSQVQLVQSGAEVKKPGE |
| | | SLKVSCKASGYTFTNYYMQWVRQAPGQ |
| | | GLEWMGIINPSGGVTSYAQKFQGRVTM |
| | | TRDTSTSTVYMELSSLRSEDTAVYYCA |
| | | RGSAYYYDFADYWGQGTLVTVSS |
| 128 | scDb_NKp46: | DIQMTQTTSSLSASLGDRVTISCRASQ |
| | | DISNYLNWYQQKPDGTVKLLIYYTSRL |
| | | HSGVPSRESGSGSGTDYSLTINNLEQE |
| | | DIATYFCQQGNTRPWTFGGGTKLEIKG |
| | | GSGGSQVQLQQSGPELVKPGASVKMSC |
| | | KASGYTFTDYVINWGKQRSGQGLEWIG |
| | | EIYPGSGTNYYNEKFKAKATLTADKSS |
| | | NIAYMQLSSLTSEDSAVYFCARRGRYG |
| | | LYAMDYWGQGTSVTVSSGGSGGSGGSG |
| | | GSGGSGGSGGSDIQMTQTTSSLSASLG |
| | | DRVTISCRASQDISNYLNWYQQKPDGT |
| | | VKLLIYYTSRLHSGVPSRFSGSGSGTD |
| | | YSLTINNLEQEDIATYFCQQGNTRPWT |
| | | FGGGTKLEIKGGSGGSQVQLQQSGPEL |
| | | VKPGASVKMSCKASGYTFTDYVINWGK |
| | | QRSGQGLEWIGEIYPGSGTNYYNEKFK |
| | | AKATLTADKSSNIAYMQLSSLTSEDSA |
| | | VYFCARRGRYGLYAMDYWGQGTSVTVS |
| | | S |
| 129 | scDb_NKG2D: | QSALTQPASVSGSPGQSITISCSGSSS |
| | | NIGNNAVNWYQQLPGKAPKLLIYYDDL |
| | | LPSGVSDRFSGSKSGTSAFLAISGLQS |
| | | EDEADYYCAAWDDSLNGPVFGGGTKLT |
| | | VLGGSGGSQVQLVESGGGLVKPGGSLR |
| | | LSCAASGFTFSSYGMHWVRQAPGKGLE |
| | | WVAFIRYDGSNKYYADSVKGRFTISRD |
| | | NSKNTLYLQMNSLRAEDTAVYYCAKDR |
| | | GLGDGTYFDYWGQGTTVTVSSGGSGGS |
| | | GGSGGSGGSGGSQSALTQPASVSGSPG |
| | | QSITISCSGSSSNIGNNAVNWYQQLPG |
| | | KAPKLLIYYDDLLPSGVSDRESGSKSG |
| | | TSAFLAISGLQSEDEADYYCAAWDDSL |
| | | NGPVFGGGTKLTVLGGSGGSQVQLVES |
| | | GGGLVKPGGSLRLSCAASGFTFSSYGM |
| | | HWVRQAPGKGLEWVAFIRYDGSNKYYA |
| | | DSVKGRFTISRDNSKNTLYLQMNSLRA |
| | | EDTAVYYCAKDRGLGDGTYFDYWGQGT |
| | | TVTVSS |
| 130 | SCFv CD16A-1: | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSGGSGGSGGSGGSGGSQVQLVQSG |
| | | AEVKKPGESLKVSCKASGYTFTSYYMH |
| | | WVRQAPGQGLEWMGAIEPMYGSTSYAQ |
| | | KFQGRVTMTRDTSTSTVYMELSSLRSE |
| | | DTAVYYCARGSAYYYDFADYWGQGTLV |
| | | TVSS |
| 131 | ScFv CD16A-2: | SYVLTQPSSVSVAPGQTARITCGGNNI |
| | | GSKSVHWYQQKPGQAPVLVIYQDKKRP |
| | | SGIPERFSGSNSGNTATLTISGTQAMD |
| | | EADYYCQVWDDYIVLFGGGTKLTVLGG |
| | | SGGSGGSGGSGGSGGSGGSQVQLVQSG |
| | | AEVKKPGESLKVSCKASGYTFTNYYMQ |
| | | WVRQAPGQGLEWMGIINPSGGVTSYAQ |
| | | KFQGRVTMTRDTSTSTVYMELSSLRSE |
| | | DTAVYYCARGSAYYYDFADYWGQGTLV |
| | | TVSS |
| 132 | scFv_NKp46: | DIQMTQTTSSLSASLGDRVTISCRASQ |
| | | DISNYLNWYQQKPDGTVKLLIYYTSRL |
| | | HSGVPSRESGSGSGTDYSLTINNLEQE |
| | | DIATYFCQQGNTRPWTFGGGTKLEIKG |
| | | GSGGSGGSGGSGGSGGSGGSQVQLQQS |
| | | GPELVKPGASVKMSCKASGYTFTDYVI |
| | | NWGKQRSGQGLEWIGEIYPGSGTNYYN |
| | | EKFKAKATLTADKSSNIAYMQLSSLTS |
| | | EDSAVYFCARRGRYGLYAMDYWGQGTS |
| | | VTVSS |
| 133 | ScFv NKG2D: | QSALTQPASVSGSPGQSITISCSGSSS |
| | | NIGNNAVNWYQQLPGKAPKLLIYYDDL |
| | | LPSGVSDRFSGSKSGTSAFLAISGLQS |
| | | EDEADYYCAAWDDSLNGPVFGGGTKLT |
| | | VLGGSGGSGGSGGSGGSGGSGGSQVQL |
| | | VESGGGLVKPGGSLRLSCAASGFTESS |
| | | YGMHWVRQAPGKGLEWVAFIRYDGSNK |
| | | YYADSVKGRFTISRDNSKNTLYLQMNS |
| | | LRAEDTAVYYCAKDRGLGDGTYFDYWG |
| | | QGTTVTVSS |
| 134 | Fab CD16A-3 | QVQLVQSGAEVKKPGESLKVSCKASGY |
| | chain 1: | TFTSYYMHWVRQAPGQGLEWMGIINPS |
| | | GGSTSYAQKFQGRVTMTRDTSTSTVYM |
| | | ELSSLRSEDTAVYYCARGSAYYYDFAD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHT |
| 135 | Fab CD16A-3 | SYVLTQPSSVSVAPGQTATISCGGHNI |
| | chain 2: | GSKNVHWYQQRPGQSPVLVIYQDNKRP |
| | | SGIPERFSGSNSGNTATLTISGTQAMD |
| | | EADYYCQVWDNYSVLFGGGTKLTVLGQ |
| | | PKAAPSVTLFPPSSEELQANKATLVCL |
| | | ISDFYPGAVTVAWKADSSPVKAGVETT |
| | | TPSKQSNNKYAASSYLSLTPEQWKSHR |
| | | SYSCQVTHEGSTVEKTVAPTECS |
| 136 | Fab BCMA chain | EVQLLESGGGLVQPGGSLRLSCAASGF |
| | 1: | TFSNYDMAWVRQAPGKGLEWVSSISTR |
| | | GDITSYRDSVKGRFTISRDNSKNTLYL |
| | | QMNSLRAEDTAVYYCARQDYYTDYMGF |
| | | AYWGQGTLVTVSSASTKGPSVFPLAPS |
| | | SKSTSGGTAALGCLVKDYFPEPVTVSW |
| | | NSGALTSGVHTFPAVLQSSGLYSLSSV |
| | | VTVPSSSLGTQTYICNVNHKPSNTKVD |
| | | KKVEPKSCDKTHT |
| 137 | Fab BCMA chain | AIQMTQSPSSLSASVGDRVTITCRASE |
| | 2: | DIYNGLAWYQQKPGKAPKLLIYGASSL |
| | | QDGVPSRFSGSGSGTEFTLTISSLQPE |
| | | DEATYYCAGPHKYPLTFGGGTKVEIKR |
| | | TVAAPSVFIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 138 | Fab CD19 | EVQLVESGGGLVKPGGSLKLSCAASGY |
| | (MOR208) chain | TFTSYVMHWVRQAPGKGLEWIGYINPY |
| | 1: | NDGTKYNEKFQGRVTISSDKSISTAYM |
| | | ELSSLRSEDTAMYYCARGTYYYGTRVF |
| | | DYWGQGTLVTVSSASTKGPSVFPLAPS |
| | | SKSTSGGTAALGCLVKDYFPEPVTVSW |
| | | NSGALTSGVHTFPAVLQSSGLYSLSSV |
| | | VTVPSSSLGTQTYICNVNHKPSNTKVD |
| | | KKVEPKSCDKTHT |
| 139 | Fab CD19 | DIVMTQSPATLSLSPGERATLSCRSSK |
| | (MOR208) chain | SLQNVNGNTYLYWFQQKPGQSPQLLIY |
| | 2: | RMSNLNSGVPDRFSGSGSGTEFTLTIS |
| | | SLEPEDFAVYYCMQHLEYPITFGAGTK |
| | | LEIKRTVAAPSVFIFPPSDEQLKSGTA |
| | | SVVCLLNNEYPREAKVQWKVDNALQSG |
| | | NSQESVTEQDSKDSTYSLSSTLTLSKA |
| | | DYEKHKVYACEVTHQGLSSPVTKSENR |
| | | GEC |
| 140 | Fab EGFR chain | QVQLQESGPGLVKPSETLSLTCTVSGG |
| | 1: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHT |
| 141 | Fab EGFR chain | QPVLTQPPSVSVAPGKTARITCGGNNI |
| | 2: | GSKSVHWYQQKPGQAPVLVIYYDSDRP |
| | | SGIPERFSGSNSGNTATLTISRVEAGD |
| | | EADYYCQVWDTSSDHVLFGGGTKLTVL |
| | | GQPKAAPSVTLEPPSSEELQANKATLV |
| | | CLISDFYPGAVTVAWKADSSPVKAGVE |
| | | TTTPSKQSNNKYAASSYLSLTPEQWKS |
| | | HRSYSCQVTHEGSTVEKTVAPTECS |
| 142 | Fab HER2 (4D5) | EVQLVESGGGLVQPGGSLRLSCAASGF |
| | chain 1: | NIKDTYIHWVRQAPGKGLEWVARIYPT |
| | | NGYTRYADSVKGRFTISADTSKNTAYL |
| | | QMNSLRAEDTAVYYCSRWGGDGFYAMD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHT |
| 143 | Fab HER2 (4D5) | DIQMTQSPSSLSASVGDRVTITCRASQ |
| | chain 2: | DVNTAVAWYQQKPGKAPKLLIYSASFL |
| | | YSGVPSRESGSRSGTDFTLTISSLQPE |
| | | DFATYYCQQHYTTPPTFGQGTKVEIKR |
| | | TVAAPSVEIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 144 | scFv_BCMA: | EVQLLESGGGLVQPGGSLRLSCAASGF |
| | | TFSNYDMAWVRQAPGKGLEWVSSISTR |
| | | GDITSYRDSVKGRFTISRDNSKNTLYL |
| | | QMNSLRAEDTAVYYCARQDYYTDYMGF |
| | | AYWGQGTLVTVSSGGSGGSGGSGGSGG |
| | | SGGSAIQMTQSPSSLSASVGDRVTITC |
| | | RASEDIYNGLAWYQQKPGKAPKLLIYG |
| | | ASSLQDGVPSRFSGSGSGTEFTLTISS |
| | | LQPEDEATYYCAGPHKYPLTFGGGTKV |
| | | EIK |
| 145 | scFv_CD19 | EVQLVESGGGLVKPGGSLKLSCAASGY |
| | (MOR208): | TFTSYVMHWVRQAPGKGLEWIGYINPY |
| | | NDGTKYNEKFQGRVTISSDKSISTAYM |
| | | ELSSLRSEDTAMYYCARGTYYYGTRVF |
| | | DYWGQGTLVTVSSGGSGGSGGSGGSGG |
| | | SGGSDIVMTQSPATLSLSPGERATLSC |
| | | RSSKSLQNVNGNTYLYWFQQKPGQSPQ |
| | | LLIYRMSNLNSGVPDRESGSGSGTEFT |
| | | LTISSLEPEDFAVYYCMQHLEYPITFG |
| | | AGTKLEIK |
| 146 | scFv_EGFR: | QVQLQESGPGLVKPSETLSLTCTVSGG |
| | | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSGGSGGSGGSGGSGGS |
| | | GGSQPVLTQPPSVSVAPGKTARITCGG |
| | | NNIGSKSVHWYQQKPGQAPVLVIYYDS |
| | | DRPSGIPERFSGSNSGNTATLTISRVE |
| | | AGDEADYYCQVWDTSSDHVLFGGGTKL |
| | | TVL |
| 147 | SCFV HER2 | EVQLVESGGGLVQPGGSLRLSCAASGF |
| | (4D5): | NIKDTYIHWVRQAPGKGLEWVARIYPT |
| | | NGYTRYADSVKGRFTISADTSKNTAYL |
| | | QMNSLRAEDTAVYYCSRWGGDGFYAMD |
| | | YWGQGTLVTVSSGGSGGSGGSGGSGGS |
| | | GGSDIQMTQSPSSLSASVGDRVTITCR |
| | | ASQDVNTAVAWYQQKPGKAPKLLIYSA |
| | | SFLYSGVPSRFSGSRSGTDFTLTISSL |
| | | QPEDFATYYCQQHYTTPPTFGQGTKVE |
| | | IK |
| 148 | Bi-scDb-Fc_01: | EVQLLESGGGLVQPGGSLRLSCAASGF |
| | | TFSNYDMAWVRQAPGKGLEWVSSISTR |
| | | GDITSYRDSVKGRFTISRDNSKNTLYL |
| | | QMNSLRAEDTAVYYCARQDYYTDYMGF |
| | | AYWGQGTLVTVSSGGGGSGGGGSGGGG |
| | | SAIQMTQSPSSLSASVGDRVTITCRAS |
| | | EDIYNGLAWYQQKPGKAPKLLIYGASS |
| | | LQDGVPSRFSGSGSGTEFTLTISSLQP |
| | | EDEATYYCAGPHKYPLTFGGGTKVEIK |
| | | EPKSCDKTHTCPPCPAPEFEGGPSVFL |
| | | FPPKPKDTLMISRTPEVTCVVVAVSHE |
| | | DPEVKFNWYVDGVEVHNAKTKPREEQY |
| | | NSTYRVVSVLTVLHQDWLNGKEYKCKV |
| | | SNKALPAPIEKTISKAKGQPREPQVYT |
| | | LPPSREEMTKNQVSLTCLVKGFYPSDI |
| | | AVEWESNGQPENNYKTTPPVLDSDGSF |
| | | FLYSKLTVDKSRWQQGNVFSCSVMHEA |
| | | LHNHYTQKSLSLSPGGGGGSGGGGSSY |
| | | VLTQPSSVSVAPGQTATISCGGHNIGS |
| | | KNVHWYQQRPGQSPVLVIYQDNKRPSG |
| | | IPERFSGSNSGNTATLTISGTQAMDEA |
| | | DYYCQVWDNYSVLFGGGTKLTVLGGSG |
| | | GSQVQLVQSGAEVKKPGESLKVSCKAS |
| | | GYTFTSYYMHWVRQAPGQGLEWMGAIE |
| | | PMYGSTSYAQKFQGRVTMTRDTSTSTV |
| | | YMELSSLRSEDTAVYYCARGSAYYYDF |
| | | ADYWGQGTLVTVSSGGSGGSGGSGGSG |
| | | GSGGSSYVLTQPSSVSVAPGQTATISC |
| | | GGHNIGSKNVHWYQQRPGQSPVLVIYQ |
| | | DNKRPSGIPERFSGSNSGNTATLTISG |
| | | TQAMDEADYYCQVWDNYSVLFGGGTKL |
| | | TVLGGSGGSQVQLVQSGAEVKKPGESL |
| | | KVSCKASGYTFTSYYMHWVRQAPGQGL |
| | | EWMGAIEPMYGSTSYAQKFQGRVTMTR |
| | | DTSTSTVYMELSSLRSEDTAVYYCARG |
| | | SAYYYDFADYWGQGTLVTVSS |
| 149 | Bi-scDb-Fc_02: | QVQLQESGPGLVKPSETLSLTCTVSGG |
| | | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSGGSGGSGGSGGSGGS |
| | | GGSQPVLTQPPSVSVAPGKTARITCGG |
| | | NNIGSKSVHWYQQKPGQAPVLVIYYDS |
| | | DRPSGIPERFSGSNSGNTATLTISRVE |
| | | AGDEADYYCQVWDTSSDHVLFGGGTKL |
| | | TVLEPKSCDKTHTCPPCPAPEFEGGPS |
| | | VFLFPPKPKDTLMISRTPEVTCVVVAV |
| | | SHEDPEVKFNWYVDGVEVHNAKTKPRE |
| | | EQYNSTYRVVSVLTVLHQDWLNGKEYK |
| | | CKVSNKALPAPIEKTISKAKGQPREPQ |
| | | VYTLPPSREEMTKNQVSLTCLVKGFYP |
| | | SDIAVEWESNGQPENNYKTTPPVLDSD |
| | | GSFFLYSKLTVDKSRWQQGNVFSCSVM |
| | | HEALHNHYTQKSLSLSPGGGGGSGGGG |
| | | SSYVLTQPSSVSVAPGQTATISCGGHN |
| | | IGSKNVHWYQQRPGQSPVLVIYQDNKR |
| | | PSGIPERFSGSNSGNTATLTISGTQAM |
| | | DEADYYCQVWDNYSVLEGGGTKLTVLG |
| | | GSGGSQVQLVQSGAEVKKPGESLKVSC |
| | | KASGYTFTSYYMHWVRQAPGQGLEWMG |
| | | AIEPMYGSTSYAQKFQGRVTMTRDTST |
| | | STVYMELSSLRSEDTAVYYCARGSAYY |
| | | YDFADYWGQGTLVTVSSGGSGGSGGSG |
| | | GSGGSGGSSYVLTQPSSVSVAPGQTAT |
| | | ISCGGHNIGSKNVHWYQQRPGQSPVLV |
| | | IYQDNKRPSGIPERFSGSNSGNTATLT |
| | | ISGTQAMDEADYYCQVWDNYSVLFGGG |
| | | TKLTVLGGSGGSQVQLVQSGAEVKKPG |
| | | ESLKVSCKASGYTFTSYYMHWVRQAPG |
| | | QGLEWMGAIEPMYGSTSYAQKFQGRVT |
| | | MTRDTSTSTVYMELSSLRSEDTAVYYC |
| | | ARGSAYYYDFADYWGQGTLVTVSS |
| 150 | aBi-scDb-Fc_01 | SYVLTQPSSVSVAPGQTATISCGGHNI |
| | chain 1: | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLFGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSSGGGG |
| | | SDKTHTCPPCPAPEFEGGPSVELFPPK |
| | | PKDTLMISRTPEVTCVVVAVSHEDPEV |
| | | KENWYVDGVEVHNAKTKPREEQYNSTY |
| | | RVVSVLTVLHQDWLNGKEYKCKVSNKA |
| | | LPAPIEKTISKAKGQPREPQVYTLPPS |
| | | REEMTKNQVSLTCLVKGFYPSDIAVEW |
| | | ESNGQPENNYKTTPPVLDSDGSFFLTS |
| | | KLTVDKSRWQQGNVESCSVMHEALHNH |
| | | YTQKSLSLSPGAAAGSHHHHHH |
| 151 | aBi-scDb-Fc_01 | SYVLTQPSSVSVAPGQTATISCGGHNI |
| | chain 2: | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLFGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSSGGGG |
| | | SDKTHTCPPCPAPEFEGGPSVFLFPPK |
| | | PKDTLMISRTPEVTCVVVAVSHEDPEV |
| | | KENWYVDGVEVHNAKTKPREEQYNSTY |
| | | RVVSVLTVLHQDWLNGKEYKCKVSNKA |
| | | LPAPIEKTISKAKGQPREPQVYTLPPS |
| | | REEMTKNQVSLYCLVKGFYPSDIAVEW |
| | | ESNGQPENNYKTTPPVLDSDGSFFLYS |
| | | KLTVDKSRWQQGNVFSCSVMHEALHNH |
| | | YTQKSLSLSPGGGGGSGGGGSEVQLLE |
| | | SGGGLVQPGGSLRLSCAASGFTFSNYD |
| | | MAWVRQAPGKGLEWVSSISTRGDITSY |
| | | RDSVKGRFTISRDNSKNTLYLQMNSLR |
| | | AEDTAVYYCARQDYYTDYMGFAYWGQG |
| | | TLVTVSSGGGGSGGGGSGGGGSAIQMT |
| | | QSPSSLSASVGDRVTITCRASEDIYNG |
| | | LAWYQQKPGKAPKLLIYGASSLQDGVP |
| | | SRFSGSGSGTEFTLTISSLQPEDEATY |
| | | YCAGPHKYPLTFGGGTKVEIK |
| 152 | aBi-scDb-Fc_02 | DKTHTCPPCPAPEFEGGPSVFLFPPKP |
| | chain 1: | KDTLMISRTPEVTCVVVAVSHEDPEVK |
| | | ENWYVDGVEVHNAKTKPREEQYNSTYR |
| | | VVSVLTVLHQDWLNGKEYKCKVSNKAL |
| | | PAPIEKTISKAKGQPREPQVYTLPPSR |
| | | EEMTKNQVSLTCLVKGFYPSDIAVEWE |
| | | SNGQPENNYKTTPPVLDSDGSFFLTSK |
| | | LTVDKSRWQQGNVFSCSVMHEALHNHY |
| | | TQKSLSLSPGGGGGSGGGGSSYVLTQP |
| | | SSVSVAPGQTATISCGGHNIGSKNVHW |
| | | YQQRPGQSPVLVIYQDNKRPSGIPERF |
| | | SGSNSGNTATLTISGTQAMDEADYYCQ |
| | | VWDNYSVLFGGGTKLTVLGGSGGSQVQ |
| | | LVQSGAEVKKPGESLKVSCKASGYTFT |
| | | SYYMHWVRQAPGQGLEWMGAIEPMYGS |
| | | TSYAQKFQGRVTMTRDTSTSTVYMELS |
| | | SLRSEDTAVYYCARGSAYYYDFADYWG |
| | | QGTLVTVSSGGSGGSGGSGGSGGSGGS |
| | | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSAAAGSHHHHHH |
| 153 | aBi-scDb-Fc_02 | EVQLLESGGGLVQPGGSLRLSCAASGF |
| | chain 2: | TFSNYDMAWVRQAPGKGLEWVSSISTR |
| | | GDITSYRDSVKGRFTISRDNSKNTLYL |
| | | QMNSLRAEDTAVYYCARQDYYTDYMGF |
| | | AYWGQGTLVTVSSGGGGGGGGSGGGGS |
| | | AIQMTQSPSSLSASVGDRVTITCRASE |
| | | DIYNGLAWYQQKPGKAPKLLIYGASSL |
| | | QDGVPSRFSGSGSGTEFTLTISSLQPE |
| | | DEATYYCAGPHKYPLTFGGGTKVEIKD |
| | | KTHTCPPCPAPEFEGGPSVFLFPPKPK |
| | | DTLMISRTPEVTCVVVAVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSRE |
| | | EMTKNQVSLYCLVKGFYPSDIAVEWES |
| | | NGQPENNYKTTPPVLDSDGSFFLYSKL |
| | | TVDKSRWQQGNVFSCSVMHEALHNHYT |
| | | QKSLSLSPGGGGGSGGGGSSYVLTQPS |
| | | SVSVAPGQTATISCGGHNIGSKNVHWY |
| | | QQRPGQSPVLVIYQDNKRPSGIPERFS |
| | | GSNSGNTATLTISGTQAMDEADYYCQV |
| | | WDNYSVLEGGGTKLTVLGGSGGSQVQL |
| | | VQSGAEVKKPGESLKVSCKASGYTFTS |
| | | YYMHWVRQAPGQGLEWMGAIEPMYGST |
| | | SYAQKFQGRVTMTRDTSTSTVYMELSS |
| | | LRSEDTAVYYCARGSAYYYDFADYWGQ |
| | | GTLVTVSSGGSGGSGGSGGSGGSGGSS |
| | | YVLTQPSSVSVAPGQTATISCGGHNIG |
| | | SKNVHWYQQRPGQSPVLVIYQDNKRPS |
| | | GIPERFSGSNSGNTATLTISGTQAMDE |
| | | ADYYCQVWDNYSVLFGGGTKLTVLGGS |
| | | GGSQVQLVQSGAEVKKPGESLKVSCKA |
| | | SGYTFTSYYMHWVRQAPGQGLEWMGAI |
| | | EPMYGSTSYAQKFQGRVTMTRDTSTST |
| | | VYMELSSLRSEDTAVYYCARGSAYYYD |
| | | FADYWGQGTLVTVSSAAAGSHHHHHH |
| 154 | aBi-scDb-Fc_03 | DKTHTCPPCPAPEFEGGPSVFLFPPKP |
| | chain 1: | KDTLMISRTPEVTCVVVAVSHEDPEVK |
| | | ENWYVDGVEVHNAKTKPREEQYNSTYR |
| | | VVSVLTVLHQDWLNGKEYKCKVSNKAL |
| | | PAPIEKTISKAKGQPREPQVYTLPPSR |
| | | EEMTKNQVSLTCLVKGFYPSDIAVEWE |
| | | SNGQPENNYKTTPPVLDSDGSFFLTSK |
| | | LTVDKSRWQQGNVFSCSVMHEALHNHY |
| | | TQKSLSLSPGGGGGSGGGGSSYVLTQP |
| | | SSVSVAPGQTATISCGGHNIGSKNVHW |
| | | YQQRPGQSPVLVIYQDNKRPSGIPERF |
| | | SGSNSGNTATLTISGTQAMDEADYYCQ |
| | | VWDNYSVLFGGGTKLTVLGGSGGSQVQ |
| | | LVQSGAEVKKPGESLKVSCKASGYTFT |
| | | SYYMHWVRQAPGQGLEWMGAIEPMYGS |
| | | TSYAQKFQGRVTMTRDTSTSTVYMELS |
| | | SLRSEDTAVYYCARGSAYYYDFADYWG |
| | | QGTLVTVSSGGSGGSGGSGGSGGSGGS |
| | | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSAAAGSHHHHHH |
| 155 | aBi-scDb-Fc_03 | SYVLTQPSSVSVAPGQTATISCGGHNI |
| | chain 2: | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLFGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSSGGGG |
| | | SDKTHTCPPCPAPEFEGGPSVELFPPK |
| | | PKDTLMISRTPEVTCVVVAVSHEDPEV |
| | | KENWYVDGVEVHNAKTKPREEQYNSTY |
| | | RVVSVLTVLHQDWLNGKEYKCKVSNKA |
| | | LPAPIEKTISKAKGQPREPQVYTLPPS |
| | | REEMTKNQVSLYCLVKGFYPSDIAVEW |
| | | ESNGQPENNYKTTPPVLDSDGSFFLYS |
| | | KLTVDKSRWQQGNVFSCSVMHEALHNH |
| | | YTQKSLSLSPGGGGGSGGGGSEVQLLE |
| | | SGGGLVQPGGSLRLSCAASGFTFSNYD |
| | | MAWVRQAPGKGLEWVSSISTRGDITSY |
| | | RDSVKGRFTISRDNSKNTLYLQMNSLR |
| | | AEDTAVYYCARQDYYTDYMGFAYWGQG |
| | | TLVTVSSGGGGSGGGGSGGGGSAIQMT |
| | | QSPSSLSASVGDRVTITCRASEDIYNG |
| | | LAWYQQKPGKAPKLLIYGASSLQDGVP |
| | | SRFSGSGSGTEFTLTISSLQPEDEATY |
| | | YCAGPHKYPLTFGGGTKVEIK |
| 156 | aBi-scDb-Fc_04 | SYVLTQPSSVSVAPGQTATISCGGHNI |
| | chain 1: | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLFGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSSGGGG |
| | | SDKTHTCPPCPAPEFEGGPSVELFPPK |
| | | PKDTLMISRTPEVTCVVVAVSHEDPEV |
| | | KENWYVDGVEVHNAKTKPREEQYNSTY |
| | | RVVSVLTVLHQDWLNGKEYKCKVSNKA |
| | | LPAPIEKTISKAKGQPREPQVYTLPPS |
| | | REEMTKNQVSLTCLVKGFYPSDIAVEW |
| | | ESNGQPENNYKTTPPVLDSDGSFFLTS |
| | | KLTVDKSRWQQGNVESCSVMHEALHNH |
| | | YTQKSLSLSPGAAAGSHHHHHH |
| 157 | aBi-scDb-Fc_04 | SYVLTQPSSVSVAPGQTATISCGGHNI |
| | chain 2: | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLFGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSSGGGG |
| | | SDKTHTCPPCPAPEFEGGPSVELFPPK |
| | | PKDTLMISRTPEVTCVVVAVSHEDPEV |
| | | KENWYVDGVEVHNAKTKPREEQYNSTY |
| | | RVVSVLTVLHQDWLNGKEYKCKVSNKA |
| | | LPAPIEKTISKAKGQPREPQVYTLPPS |
| | | REEMTKNQVSLYCLVKGFYPSDIAVEW |
| | | ESNGQPENNYKTTPPVLDSDGSFFLYS |
| | | KLTVDKSRWQQGNVESCSVMHEALHNH |
| | | YTQKSLSLSPGGGGGSGGGGSQVQLQE |
| | | SGPGLVKPSETLSLTCTVSGGSVSSGS |
| | | YYWSWIRQPPGKGLEWIGYIYYSGSTN |
| | | YNPSLKSRVTISVDTSKNQFSLKLSSV |
| | | TAADTAVYYCARNPISIPAFDIWGQGT |
| | | MVTVSSGGSGGSGGSGGSGGSGGSQPV |
| | | LTQPPSVSVAPGKTARITCGGNNIGSK |
| | | SVHWYQQKPGQAPVLVIYYDSDRPSGI |
| | | PERFSGSNSGNTATLTISRVEAGDEAD |
| | | YYCQVWDTSSDHVLFGGGTKLTVLGAA |
| | | EPEA |
| 158 | aBi-scDb-Fc_05 | DKTHTCPPCPAPEFEGGPSVFLFPPKP |
| | chain 1: | KDTLMISRTPEVTCVVVAVSHEDPEVK |
| | | ENWYVDGVEVHNAKTKPREEQYNSTYR |
| | | VVSVLTVLHQDWLNGKEYKCKVSNKAL |
| | | PAPIEKTISKAKGQPREPQVYTLPPSR |
| | | EEMTKNQVSLTCLVKGFYPSDIAVEWE |
| | | SNGQPENNYKTTPPVLDSDGSFFLTSK |
| | | LTVDKSRWQQGNVFSCSVMHEALHNHY |
| | | TQKSLSLSPGGGGGSGGGGSSYVLTQP |
| | | SSVSVAPGQTATISCGGHNIGSKNVHW |
| | | YQQRPGQSPVLVIYQDNKRPSGIPERF |
| | | SGSNSGNTATLTISGTQAMDEADYYCQ |
| | | VWDNYSVLFGGGTKLTVLGGSGGSQVQ |
| | | LVQSGAEVKKPGESLKVSCKASGYTFT |
| | | SYYMHWVRQAPGQGLEWMGAIEPMYGS |
| | | TSYAQKFQGRVTMTRDTSTSTVYMELS |
| | | SLRSEDTAVYYCARGSAYYYDFADYWG |
| | | QGTLVTVSSGGSGGSGGSGGSGGSGGS |
| | | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSAAAGSHHHHHH |
| 159 | aBi-scDb-Fc_05 | QVQLQESGPGLVKPSETLSLTCTVSGG |
| | chain 2: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSGGSGGSGGSGGSGGS |
| | | GGSQPVLTQPPSVSVAPGKTARITCGG |
| | | NNIGSKSVHWYQQKPGQAPVLVIYYDS |
| | | DRPSGIPERFSGSNSGNTATLTISRVE |
| | | AGDEADYYCQVWDTSSDHVLFGGGTKL |
| | | TVLDKTHTCPPCPAPEFEGGPSVFLFP |
| | | PKPKDTLMISRTPEVTCVVVAVSHEDP |
| | | EVKFNWYVDGVEVHNAKTKPREEQYNS |
| | | TYRVVSVLTVLHQDWLNGKEYKCKVSN |
| | | KALPAPIEKTISKAKGQPREPQVYTLP |
| | | PSREEMTKNQVSLYCLVKGFYPSDIAV |
| | | EWESNGQPENNYKTTPPVLDSDGSFFL |
| | | YSKLTVDKSRWQQGNVFSCSVMHEALH |
| | | NHYTQKSLSLSPGGGGGSGGGGSSYVL |
| | | TQPSSVSVAPGQTATISCGGHNIGSKN |
| | | VHWYQQRPGQSPVLVIYQDNKRPSGIP |
| | | ERFSGSNSGNTATLTISGTQAMDEADY |
| | | YCQVWDNYSVLFGGGTKLTVLGGSGGS |
| | | QVQLVQSGAEVKKPGESLKVSCKASGY |
| | | TFTSYYMHWVRQAPGQGLEWMGAIEPM |
| | | YGSTSYAQKFQGRVTMTRDTSTSTVYM |
| | | ELSSLRSEDTAVYYCARGSAYYYDFAD |
| | | YWGQGTLVTVSSGGSGGSGGSGGSGGS |
| | | GGSSYVLTQPSSVSVAPGQTATISCGG |
| | | HNIGSKNVHWYQQRPGQSPVLVIYQDN |
| | | KRPSGIPERFSGSNSGNTATLTISGTQ |
| | | AMDEADYYCQVWDNYSVLFGGGTKLTV |
| | | LGGSGGSQVQLVQSGAEVKKPGESLKV |
| | | SCKASGYTFTSYYMHWVRQAPGQGLEW |
| | | MGAIEPMYGSTSYAQKFQGRVTMTRDT |
| | | STSTVYMELSSLRSEDTAVYYCARGSA |
| | | YYYDFADYWGQGTLVTVSSGAAEPEA |
| 160 | aBi-scDb-Fc_06 | DKTHTCPPCPAPEFEGGPSVFLFPPKP |
| | chain 1: | KDTLMISRTPEVTCVVVAVSHEDPEVK |
| | | ENWYVDGVEVHNAKTKPREEQYNSTYR |
| | | VVSVLTVLHQDWLNGKEYKCKVSNKAL |
| | | PAPIEKTISKAKGQPREPQVYTLPPSR |
| | | EEMTKNQVSLTCLVKGFYPSDIAVEWE |
| | | SNGQPENNYKTTPPVLDSDGSFFLTSK |
| | | LTVDKSRWQQGNVFSCSVMHEALHNHY |
| | | TQKSLSLSPGGGGGSGGGGSSYVLTQP |
| | | SSVSVAPGQTATISCGGHNIGSKNVHW |
| | | YQQRPGQSPVLVIYQDNKRPSGIPERF |
| | | SGSNSGNTATLTISGTQAMDEADYYCQ |
| | | VWDNYSVLFGGGTKLTVLGGSGGSQVQ |
| | | LVQSGAEVKKPGESLKVSCKASGYTFT |
| | | SYYMHWVRQAPGQGLEWMGAIEPMYGS |
| | | TSYAQKFQGRVTMTRDTSTSTVYMELS |
| | | SLRSEDTAVYYCARGSAYYYDFADYWG |
| | | QGTLVTVSSGGSGGSGGSGGSGGSGGS |
| | | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSAAAGSHHHHHH |
| 161 | aBi-scDb-Fc_06 | SYVLTQPSSVSVAPGQTATISCGGHNI |
| | chain 2: | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLFGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSSGGGG |
| | | SDKTHTCPPCPAPEFEGGPSVELFPPK |
| | | PKDTLMISRTPEVTCVVVAVSHEDPEV |
| | | KENWYVDGVEVHNAKTKPREEQYNSTY |
| | | RVVSVLTVLHQDWLNGKEYKCKVSNKA |
| | | LPAPIEKTISKAKGQPREPQVYTLPPS |
| | | REEMTKNQVSLYCLVKGFYPSDIAVEW |
| | | ESNGQPENNYKTTPPVLDSDGSFFLYS |
| | | KLTVDKSRWQQGNVESCSVMHEALHNH |
| | | YTQKSLSLSPGGGGGSGGGGSQVQLQE |
| | | SGPGLVKPSETLSLTCTVSGGSVSSGS |
| | | YYWSWIRQPPGKGLEWIGYIYYSGSTN |
| | | YNPSLKSRVTISVDTSKNQFSLKLSSV |
| | | TAADTAVYYCARNPISIPAFDIWGQGT |
| | | MVTVSSGGSGGSGGSGGSGGSGGSQPV |
| | | LTQPPSVSVAPGKTARITCGGNNIGSK |
| | | SVHWYQQKPGQAPVLVIYYDSDRPSGI |
| | | PERFSGSNSGNTATLTISRVEAGDEAD |
| | | YYCQVWDTSSDHVLFGGGTKLTVLGAA |
| | | EPEA |
| 162 | Bi-scDb-IgAb_06 | QVQLQESGPGLVKPSETLSLTCTVSGG |
| | chain 1: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | FLFPPKPKDTLMISRTPEVTCVVVAVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVFSCSVMH |
| | | EALHNHYTQKSLSLSPGGGGGSGGGGS |
| | | SYVLTQPSSVSVAPGQTATISCGGHNI |
| | | GSKNVHWYQQRPGQSPVLVIYQDNKRP |
| | | SGIPERFSGSNSGNTATLTISGTQAMD |
| | | EADYYCQVWDNYSVLFGGGTKLTVLGG |
| | | SGGSQVQLVQSGAEVKKPGESLKVSCK |
| | | ASGYTFTSYYMHWVRQAPGQGLEWMGA |
| | | IEPMYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYVLTQPSSVSVAPGQTATI |
| | | SCGGHNIGSKNVHWYQQRPGQSPVLVI |
| | | YQDNKRPSGIPERFSGSNSGNTATLTI |
| | | SGTQAMDEADYYCQVWDNYSVLEGGGT |
| | | KLTVLGGSGGSQVQLVQSGAEVKKPGE |
| | | SLKVSCKASGYTFTSYYMHWVRQAPGQ |
| | | GLEWMGAIEPMYGSTSYAQKFQGRVTM |
| | | TRDTSTSTVYMELSSLRSEDTAVYYCA |
| | | RGSAYYYDFADYWGQGTLVTVSS |
| 163 | Bi-scDb-IgAb_06 | QPVLTQPPSVSVAPGKTARITCGGNNI |
| | chain 2: | GSKSVHWYQQKPGQAPVLVIYYDSDRP |
| | | SGIPERFSGSNSGNTATLTISRVEAGD |
| | | EADYYCQVWDTSSDHVLFGGGTKLTVL |
| | | GQPKAAPSVTLEPPSSEELQANKATLV |
| | | CLISDFYPGAVTVAWKADSSPVKAGVE |
| | | TTTPSKQSNNKYAASSYLSLTPEQWKS |
| | | HRSYSCQVTHEGSTVEKTVAPTECS |
| 164 | IgAb_49 chain | QVQLQESGPGLVKPSETLSLTCTVSGG |
| | 1: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPELLGGPSV |
| | | ELFPPKPKDTLMISRTPEVTCVVVDVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVESCSVMH |
| | | EALHNHYTQKSLSLSPG |
| 165 | IgAb_49 chain | QPVLTQPPSVSVAPGKTARITCGGNNI |
| | 2: | GSKSVHWYQQKPGQAPVLVIYYDSDRP |
| | | SGIPERFSGSNSGNTATLTISRVEAGD |
| | | EADYYCQVWDTSSDHVLFGGGTKLTVL |
| | | GQPKAAPSVTLEPPSSEELQANKATLV |
| | | CLISDFYPGAVTVAWKADSSPVKAGVE |
| | | TTTPSKQSNNKYAASSYLSLTPEQWKS |
| | | HRSYSCQVTHEGSTVEKTVAPTECS |
| 166 | IgAb_51 chain | EVQLLESGGGLVQPGGSLRLSCAVSGF |
| | 1: | TENSFAMSWVRQAPGKGLEWVSAISGS |
| | | GGGTYYADSVKGRFTISRDNSKNTLYL |
| | | QMNSLRAEDTAVYFCAKDKILWFGEPV |
| | | FDYWGQGTLVTVSSASTKGPSVFPLAP |
| | | SSKSTSGGTAALGCLVKDYFPEPVTVS |
| | | WNSGALTSGVHTFPAVLQSSGLYSLSS |
| | | VVTVPSSSLGTQTYICNVNHKPSNTKV |
| | | DKRVEPKSCDKTHTCPPCPAPELLGGP |
| | | SVFLFPPKPKDTLMISRTPEVTCVVVD |
| | | VSHEDPEVKENWYVDGVEVHNAKTKPR |
| | | EEQYNSTYRVVSVLTVLHQDWLNGKEY |
| | | KCKVSNKALPAPIEKTISKAKGQPREP |
| | | QVYTLPPSREEMTKNQVSLTCLVKGFY |
| | | PSDIAVEWESNGQPENNYKTTPPVLDS |
| | | DGSFFLYSKLTVDKSRWQQGNVFSCSV |
| | | MHEALHNHYTQKSLSLSPGK |
| 167 | IgAb_51 chain | EIVLTQSPATLSLSPGERATLSCRASQ |
| | 2: | SVSSYLAWYQQKPGQAPRLLIYDASNR |
| | | ATGIPARFSGSGSGTDFTLTISSLEPE |
| | | DFAVYYCQQRSNWPPTFGQGTKVEIKR |
| | | TVAAPSVFIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 168 | IgAb_53 chain | QVQLQESGPGLVKPSETLSLTCTVSGG |
| | 1: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPELLGGPDV |
| | | ELFPPKPKDTLMISRTPEVTCVVVDVS |
| | | HEDPEVKENWYVDGVEVHNAKTKPREE |
| | | QYNSTYRVVSVLTVLHQDWLNGKEYKC |
| | | KVSNKALPAPEEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVESCSVMH |
| | | EALHNHYTQKSLSLSPG |
| 169 | IgAb_53 chain | QPVLTQPPSVSVAPGKTARITCGGNNI |
| | 2: | GSKSVHWYQQKPGQAPVLVIYYDSDRP |
| | | SGIPERFSGSNSGNTATLTISRVEAGD |
| | | EADYYCQVWDTSSDHVLFGGGTKLTVL |
| | | GQPKAAPSVTLFPPSSEELQANKATLV |
| | | CLISDFYPGAVTVAWKADSSPVKAGVE |
| | | TTTPSKQSNNKYAASSYLSLTPEQWKS |
| | | HRSYSCQVTHEGSTVEKTVAPTECS |
| 170 | scFv-IgAb_02 | QVQLVQSGAEVKKPGESLKVSCKASGY |
| | chain 1: | TFTSYYMHWVRQAPGQGLEWMGIINPS |
| | | GGSTSYAQKFQGRVTMTRDTSTSTVYM |
| | | ELSSLRSEDTAVYYCARGSAYYYDFAD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | FLFPPKPKDTLMISRTPEVTCVVVAVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVESCSVMH |
| | | EALHNHYTQKSLSLSPGGGGGSGGGGS |
| | | QVQLQESGPGLVKPSETLSLTCTVSGG |
| | | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSGGSGGSGGSGGSGGS |
| | | GGSQPVLTQPPSVSVAPGKTARITCGG |
| | | NNIGSKSVHWYQQKPGQAPVLVIYYDS |
| | | DRPSGIPERFSGSNSGNTATLTISRVE |
| | | AGDEADYYCQVWDTSSDHVLFGGGTKL |
| | | TVL |
| 171 | scFv-IgAb_02 | SYVLTQPSSVSVAPGQTATISCGGHNI |
| | chain 2: | GSKNVHWYQQRPGQSPVLVIYQDNKRP |
| | | SGIPERFSGSNSGNTATLTISGTQAMD |
| | | EADYYCQVWDNYSVLFGGGTKLTVLGQ |
| | | PKAAPSVTLFPPSSEELQANKATLVCL |
| | | ISDFYPGAVTVAWKADSSPVKAGVETT |
| | | TPSKQSNNKYAASSYLSLTPEQWKSHR |
| | | SYSCQVTHEGSTVEKTVAPTECS |
| 172 | scFv-IgAb_18 | EVQLLESGGGLVQPGGSLRLSCAASGF |
| | chain 1: | TFSNYDMAWVRQAPGKGLEWVSSISTR |
| | | GDITSYRDSVKGRFTISRDNSKNTLYL |
| | | QMNSLRAEDTAVYYCARQDYYTDYMGF |
| | | AYWGQGTLVTVSSASTKGPSVFPLAPS |
| | | SKSTSGGTAALGCLVKDYFPEPVTVSW |
| | | NSGALTSGVHTFPAVLQSSGLYSLSSV |
| | | VTVPSSSLGTQTYICNVNHKPSNTKVD |
| | | KKVEPKSCDKTHTCPPCPAPEFEGGPS |
| | | VFLFPPKPKDTLMISRTPEVTCVVVAV |
| | | SHEDPEVKFNWYVDGVEVHNAKTKPRE |
| | | EQYNSTYRVVSVLTVLHQDWLNGKEYK |
| | | CKVSNKALPAPIEKTISKAKGQPREPQ |
| | | VYTLPPSREEMTKNQVSLTCLVKGFYP |
| | | SDIAVEWESNGQPENNYKTTPPVLDSD |
| | | GSFFLYSKLTVDKSRWQQGNVFSCSVM |
| | | HEALHNHYTQKSLSLSPGGGGGSGGGG |
| | | SGGGGSGGGGSGGGGSGGGGSSYVLTQ |
| | | PSSVSVAPGQTATISCGGHNIGSKNVH |
| | | WYQQRPGQSPVLVIYQDNKRPSGIPER |
| | | FSGSNSGNTATLTISGTQAMDEADYYC |
| | | QVWDNYSVLFGGGTKLTVLGGSGGSGG |
| | | SGGSGGSGGSGGSQVQLVQSGAEVKKP |
| | | GESLKVSCKASGYTFTSYYMHWVRQAP |
| | | GQGLEWMGAIEPMYGSTSYAQKFQGRV |
| | | TMTRDTSTSTVYMELSSLRSEDTAVYY |
| | | CARGSAYYYDFADYWGQGTLVTVSS |
| 173 | scFv-IgAb_18 | AIQMTQSPSSLSASVGDRVTITCRASE |
| | chain 2: | DIYNGLAWYQQKPGKAPKLLIYGASSL |
| | | QDGVPSRFSGSGSGTEFTLTISSLQPE |
| | | DEATYYCAGPHKYPLTFGGGTKVEIKR |
| | | TVAAPSVFIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 174 | scFv-IgAb_43 | QVQLQESGPGLVKPSETLSLTCTVSGG |
| | chain 1: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | FLFPPKPKDTLMISRTPEVTCVVVAVS |
| | | HEDPEVKFNWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVESCSVMH |
| | | EALHNHYTQKSLSLSPGGGGGSGGGGS |
| | | GGGGSGGGGSGGGGSGGGGSSYVLTQP |
| | | SSVSVAPGQTATISCGGHNIGSKNVHW |
| | | YQQRPGQSPVLVIYQDNKRPSGIPERF |
| | | SGSNSGNTATLTISGTQAMDEADYYCQ |
| | | VWDNYSVLEGGGTKLTVLGGSGGSGGS |
| | | GGSGGSGGSGGSQVQLVQSGAEVKKPG |
| | | ESLKVSCKASGYTFTSYYMHWVRQAPG |
| | | QGLEWMGAIEPMYGSTSYAQKFQGRVT |
| | | MTRDTSTSTVYMELSSLRSEDTAVYYC |
| | | ARGSAYYYDFADYWGQGTLVTVSS |
| 175 | scFv-IgAb_43 | QPVLTQPPSVSVAPGKTARITCGGNNI |
| | chain 2 | GSKSVHWYQQKPGQAPVLVIYYDSDRP |
| | | SGIPERFSGSNSGNTATLTISRVEAGD |
| | | EADYYCQVWDTSSDHVLFGGGTKLTVL |
| | | GQPKAAPSVTLEPPSSEELQANKATLV |
| | | CLISDFYPGAVTVAWKADSSPVKAGVE |
| | | TTTPSKQSNNKYAASSYLSLTPEQWKS |
| | | HRSYSCQVTHEGSTVEKTVAPTECS |
| 176 | scFv-IgAb_59 | EVQLVESGGGLVKPGGSLKLSCAASGY |
| | chain 1: | TFTSYVMHWVRQAPGKGLEWIGYINPY |
| | | NDGTKYNEKFQGRVTISSDKSISTAYM |
| | | ELSSLRSEDTAMYYCARGTYYYGTRVF |
| | | DYWGQGTLVTVSSASTKGPSVFPLAPS |
| | | SKSTSGGTAALGCLVKDYFPEPVTVSW |
| | | NSGALTSGVHTFPAVLQSSGLYSLSSV |
| | | VTVPSSSLGTQTYICNVNHKPSNTKVD |
| | | KKVEPKSCDKTHTCPPCPAPEFEGGPS |
| | | VFLFPPKPKDTLMISRTPEVTCVVVAV |
| | | SHEDPEVKFNWYVDGVEVHNAKTKPRE |
| | | EQYNSTYRVVSVLTVLHQDWLNGKEYK |
| | | CKVSNKALPAPIEKTISKAKGQPREPQ |
| | | VYTLPPSREEMTKNQVSLTCLVKGFYP |
| | | SDIAVEWESNGQPENNYKTTPPVLDSD |
| | | GSFFLYSKLTVDKSRWQQGNVFSCSVM |
| | | HEALHNHYTQKSLSLSPGGGGGSGGGG |
| | | SGGGGSGGGGSGGGGSGGGGSSYVLTQ |
| | | PSSVSVAPGQTATISCGGHNIGSKNVH |
| | | WYQQRPGQSPVLVIYQDNKRPSGIPER |
| | | FSGSNSGNTATLTISGTQAMDEADYYC |
| | | QVWDNYSVLFGGGTKLTVLGGSGGSGG |
| | | SGGSGGSGGSGGSQVQLVQSGAEVKKP |
| | | GESLKVSCKASGYTFTSYYMHWVRQAP |
| | | GQGLEWMGAIEPMYGSTSYAQKFQGRV |
| | | TMTRDTSTSTVYMELSSLRSEDTAVYY |
| | | CARGSAYYYDFADYWGQGTLVTVSS |
| 177 | scFv-IgAb_59 | DIVMTQSPATLSLSPGERATLSCRSSK |
| | chain 2: | SLQNVNGNTYLYWFQQKPGQSPQLLIY |
| | | RMSNLNSGVPDRFSGSGSGTEFTLTIS |
| | | SLEPEDFAVYYCMQHLEYPITFGAGTK |
| | | LEIKRTVAAPSVFIFPPSDEQLKSGTA |
| | | SVVCLLNNEYPREAKVQWKVDNALQSG |
| | | NSQESVTEQDSKDSTYSLSSTLTLSKA |
| | | DYEKHKVYACEVTHQGLSSPVTKSFNR |
| | | GEC |
| 178 | scFv-IgAb_167 | QVQLQESGPGLVKPSETLSLTCTVSGG |
| | chain 1: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | FLFPPKPKDTLMISRTPEVTCVVVAVS |
| | | HEDPEVKFNWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVFSCSVMH |
| | | EALHNHYTQKSLSLSPGGGGGSGGGGS |
| | | GGGGSGGGGSGGGGSGGGGSSYVLTQP |
| | | SSVSVAPGQTARITCGGNNIGSKSVHW |
| | | YQQKPGQAPVLVIYQDKKRPSGIPERF |
| | | SGSNSGNTATLTISGTQAMDEADYYCQ |
| | | VWDDYIVLEGGGTKLTVLGGSGGSGGS |
| | | GGSGGSGGSGGSQVQLVQSGAEVKKPG |
| | | ESLKVSCKASGYTFTNYYMQWVRQAPG |
| | | QGLEWMGIINPSGGVTSYAQKFQGRVT |
| | | MTRDTSTSTVYMELSSLRSEDTAVYYC |
| | | ARGSAYYYDFADYWGQGTLVTVSS |
| 179 | ScFv-IgAb_167 | QPVLTQPPSVSVAPGKTARITCGGNNI |
| | chain 2: | GSKSVHWYQQKPGQAPVLVIYYDSDRP |
| | | SGIPERFSGSNSGNTATLTISRVEAGD |
| | | EADYYCQVWDTSSDHVLFGGGTKLTVL |
| | | GQPKAAPSVTLEPPSSEELQANKATLV |
| | | CLISDFYPGAVTVAWKADSSPVKAGVE |
| | | TTTPSKQSNNKYAASSYLSLTPEQWKS |
| | | HRSYSCQVTHEGSTVEKTVAPTECS |
| 180 | AIG-2scDb_06 | EVQLVESGGGLVQPGGSLRLSCAASGF |
| | chain 1: | NIKDTYIHWVRQAPGKGLEWVARIYPT |
| | | NGYTRYADSVKGRFTISADTSKNTAYL |
| | | QMNSLRAEDTAVYYCSRWGGDGFYAMD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | ELFPPKPKDTLMISRTPEVTCVVVAVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLSCAVKGFYPS |
| | | DIAVEWESNGQPENNYKTTPPVLDSDG |
| | | SFFLVSKLTVDKSRWQQGNVESCSVMH |
| | | EALHNHYTQKSLSLSPGGGGGSGGGGS |
| | | DIQMTQTTSSLSASLGDRVTISCRASQ |
| | | DISNYLNWYQQKPDGTVKLLIYYTSRL |
| | | HSGVPSRFSGSGSGTDYSLTINNLEQE |
| | | DIATYFCQQGNTRPWTFGGGTKLEIKG |
| | | GSGGSQVQLQQSGPELVKPGASVKMSC |
| | | KASGYTFTDYVINWGKQRSGQGLEWIG |
| | | EIYPGSGTNYYNEKFKAKATLTADKSS |
| | | NIAYMQLSSLTSEDSAVYFCARRGRYG |
| | | LYAMDYWGQGTSVTVSSGGSGGSGGSG |
| | | GSGGSGGSGGSDIQMTQTTSSLSASLG |
| | | DRVTISCRASQDISNYLNWYQQKPDGT |
| | | VKLLIYYTSRLHSGVPSRFSGSGSGTD |
| | | YSLTINNLEQEDIATYFCQQGNTRPWT |
| | | FGGGTKLEIKGGSGGSQVQLQQSGPEL |
| | | VKPGASVKMSCKASGYTFTDYVINWGK |
| | | QRSGQGLEWIGEIYPGSGTNYYNEKFK |
| | | AKATLTADKSSNIAYMQLSSLTSEDSA |
| | | VYFCARRGRYGLYAMDYWGQGTSVTVS |
| | | S |
| 181 | AIG-2scDb_06 | DIQMTQSPSSLSASVGDRVTITCRASQ |
| | chain 2: | DVNTAVAWYQQKPGKAPKLLIYSASFL |
| | | YSGVPSRESGSRSGTDFTLTISSLQPE |
| | | DFATYYCQQHYTTPPTFGQGTKVEIKR |
| | | TVAAPSVFIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 182 | AIG-2scDb_06 | EVQLVESGGGLVQPGGSLRLSCAASGF |
| | chain 3: | NIKDTYIHWVRQAPGKGLEWVARIYPT |
| | | NGYTRYADSVKGRFTISADTSKNTAYL |
| | | QMNSLRAEDTAVYYCSRWGGDGFYAMD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | FLFPPKPKDTLMISRTPEVTCVVVAVS |
| | | HEDPEVKFNWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLWCLVKGFYPS |
| | | DIAVEWESNGQPENNYKTTPPVLDSDG |
| | | SFFLYSKLTVDKSRWQQGNVFSCSVMH |
| | | EALHNHYTQKSLSLSPGGGGGSGGGGS |
| | | DIQMTQTTSSLSASLGDRVTISCRASQ |
| | | DISNYLNWYQQKPDGTVKLLIYYTSRL |
| | | HSGVPSRFSGSGSGTDYSLTINNLEQE |
| | | DIATYFCQQGNTRPWTFGGGTKLEIKG |
| | | GSGGSQVQLQQSGPELVKPGASVKMSC |
| | | KASGYTFTDYVINWGKQRSGQGLEWIG |
| | | EIYPGSGTNYYNEKFKAKATLTADKSS |
| | | NIAYMQLSSLTSEDSAVYFCARRGRYG |
| | | LYAMDYWGQGTSVTVSSGGSGGSGGSG |
| | | GSGGSGGSGGSDIQMTQTTSSLSASLG |
| | | DRVTISCRASQDISNYLNWYQQKPDGT |
| | | VKLLIYYTSRLHSGVPSRFSGSGSGTD |
| | | YSLTINNLEQEDIATYFCQQGNTRPWT |
| | | FGGGTKLEIKGGSGGSQVQLQQSGPEL |
| | | VKPGASVKMSCKASGYTFTDYVINWGK |
| | | QRSGQGLEWIGEIYPGSGTNYYNEKFK |
| | | AKATLTADKSSNIAYMQLSSLTSEDSA |
| | | VYFCARRGRYGLYAMDYWGQGTSVTVS |
| | | S |
| 183 | AIG-2scDb_06 | DIQMTQSPSSLSASVGDRVTITCRASQ |
| | chain 4: | DVNTAVAWYQQKPGKAPKLLIYSASFL |
| | | YSGVPSRESGSRSGTDFTLTISSLQPE |
| | | DFATYYCQQHYTTPPTFGQGTKVEIKR |
| | | TVAAPSVEIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 184 | AIG-2scFv_27 | EVQLVESGGGLVQPGGSLRLSCAASGF |
| | chain 1: | NIKDTYIHWVRQAPGKGLEWVARIYPT |
| | | NGYTRYADSVKGRFTISADTSKNTAYL |
| | | QMNSLRAEDTAVYYCSRWGGDGFYAMD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | FLFPPKPKDTLMISRTPEVTCVVVAVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLSCAVKGFYPS |
| | | DIAVEWESNGQPENNYKTTPPVLDSDG |
| | | SFFLVSKLTVDKSRWQQGNVESCSVMH |
| | | EALHNHYTQKSLSLSPGGGGGSGGGGS |
| | | GGGGSGGGGSGGGGSGGGGSDIQMTQT |
| | | TSSLSASLGDRVTISCRASQDISNYLN |
| | | WYQQKPDGTVKLLIYYTSRLHSGVPSR |
| | | FSGSGSGTDYSLTINNLEQEDIATYFC |
| | | QQGNTRPWTFGGGTKLEIKGGSGGSGG |
| | | SGGSGGSGGSGGSQVQLQQSGPELVKP |
| | | GASVKMSCKASGYTFTDYVINWGKQRS |
| | | GQGLEWIGEIYPGSGTNYYNEKFKAKA |
| | | TLTADKSSNIAYMQLSSLTSEDSAVYF |
| | | CARRGRYGLYAMDYWGQGTSVTVSS |
| 185 | AIG-2scFv_27 | DIQMTQSPSSLSASVGDRVTITCRASQ |
| | chain 2: | DVNTAVAWYQQKPGKAPKLLIYSASFL |
| | | YSGVPSRESGSRSGTDFTLTISSLQPE |
| | | DFATYYCQQHYTTPPTFGQGTKVEIKR |
| | | TVAAPSVEIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 186 | AIG-2scFv_27 | EVQLVESGGGLVQPGGSLRLSCAASGF |
| | chain 3: | NIKDTYIHWVRQAPGKGLEWVARIYPT |
| | | NGYTRYADSVKGRFTISADTSKNTAYL |
| | | QMNSLRAEDTAVYYCSRWGGDGFYAMD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | ELFPPKPKDTLMISRTPEVTCVVVAVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLWCLVKGFYPS |
| | | DIAVEWESNGQPENNYKTTPPVLDSDG |
| | | SFFLYSKLTVDKSRWQQGNVFSCSVMH |
| | | EALHNHYTQKSLSLSPGGGGGSGGGGG |
| | | GGGSGGGGSGGGGSGGGGSDIQMTQTT |
| | | SSLSASLGDRVTISCRASQDISNYLNW |
| | | YQQKPDGTVKLLIYYTSRLHSGVPSRF |
| | | SGSGSGTDYSLTINNLEQEDIATYFCQ |
| | | QGNTRPWTFGGGTKLEIKGGSGGSGGS |
| | | GGSGGSGGSGGSQVQLQQSGPELVKPG |
| | | ASVKMSCKASGYTFTDYVINWGKQRSG |
| | | QGLEWIGEIYPGSGTNYYNEKFKAKAT |
| | | LTADKSSNIAYMQLSSLTSEDSAVYFC |
| | | ARRGRYGLYAMDYWGQGTSVTVSS |
| 187 | AIG-2scFv_27 | DIQMTQSPSSLSASVGDRVTITCRASQ |
| | chain 4: | DVNTAVAWYQQKPGKAPKLLIYSASFL |
| | | YSGVPSRESGSRSGTDFTLTISSLQPE |
| | | DFATYYCQQHYTTPPTFGQGTKVEIKR |
| | | TVAAPSVEIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 188 | scFv-IgAb_47 | QVQLVQSGAEVKKPGESLKVSCKASGY |
| | chain 1: | TFTSYYMHWVRQAPGQGLEWMGAIEPM |
| | | YGSTSYAQKFQGRVTMTRDTSTSTVYM |
| | | ELSSLRSEDTAVYYCARGSAYYYDFAD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | ELFPPKPKDTLMISRTPEVTCVVVAVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVESCSVMH |
| | | EALHNHYTQKSLSLSPGGGGGSGGGGS |
| | | QVQLQESGPGLVKPSETLSLTCTVSGG |
| | | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSGGSGGSGGSGGSGGS |
| | | GGSQPVLTQPPSVSVAPGKTARITCGG |
| | | NNIGSKSVHWYQQKPGQAPVLVIYYDS |
| | | DRPSGIPERFSGSNSGNTATLTISRVE |
| | | AGDEADYYCQVWDTSSDHVLFGGGTKL |
| | | TVL |
| 189 | scFv-IgAb_47 | SYVLTQPSSVSVAPGQTATISCGGHNI |
| | chain 2: | GSKNVHWYQQRPGQSPVLVIYQDNKRP |
| | | SGIPERFSGSNSGNTATLTISGTQAMD |
| | | EADYYCQVWDNYSVLFGGGTKLTVLGQ |
| | | PKAAPSVTLFPPSSEELQANKATLVCL |
| | | ISDFYPGAVTVAWKADSSPVKAGVETT |
| | | TPSKQSNNKYAASSYLSLTPEQWKSHR |
| | | SYSCQVTHEGSTVEKTVAPTECS |
| 190 | scFv-Fc- | QVQLQESGPGLVKPSETLSLTCTVSGG |
| 191 | scDb_04: | SVSSGSYYWSWIRQPPGKGLEWIGYIY |
| | | YSGSTNYNPSLKSRVTISVDTSKNQFS |
| | | LKLSSVTAADTAVYYCARNPISIPAFD |
| | | IWGQGTMVTVSSGGSGGSGGSGGSGGS |
| | | GGSQPVLTQPPSVSVAPGKTARITCGG |
| | | NNIGSKSVHWYQQKPGQAPVLVIYYDS |
| | | DRPSGIPERFSGSNSGNTATLTISRVE |
| | | AGDEADYYCQVWDTSSDHVLFGGGTKL |
| | | TVLGGGGSDKTHTCPPCPAPEFEGGPS |
| | | VFLFPPKPKDTLMISRTPEVTCVVVAV |
| | | SHEDPEVKFNWYVDGVEVHNAKTKPRE |
| | | EQYGSTYRVVSVLTVLHQDWLNGKEYK |
| | | CKVSNKALPAPIEKTISKAKGQPREPQ |
| | | VYTLPPSREEMTKNQVSLTCLVKGFYP |
| | | SDIAVEWESNGQPENNYKTTPPVLDSD |
| | | GSFFLYSKLTVDKSRWQQGNVFSCSVM |
| | | HEALHNHYTQKSLSLSPGGGGGSGGGG |
| | | SSYELTQPLSVSVALGQTARITCGGHN |
| | | IGSKNVHWYQQKPGQAPVLVIYQDNKR |
| | | PSGIPERFSGSNSGNTATLTISRAQAG |
| | | DEADYYCQVWDNYNVLEGCGTKLTVLG |
| | | GSGGSQVQLVQSGAEVKKPGASVKVSC |
| | | KASGYTFTSYYMHWVRQAPGQCLEWMG |
| | | AIEPTYGSTSYAQKFQGRVTMTRDTST |
| | | STVYMELSSLRSEDTAVYYCARGSAYY |
| | | YDFADYWGQGTLVTVSSGGSGGSGGSG |
| | | GSGGSGGSSYELTQPLSVSVALGQTAR |
| | | ITCGGHNIGSKNVHWYQQKPGQAPVLV |
| | | IYQDNKRPSGIPERFSGSNSGNTATLT |
| | | ISRAQAGDEADYYCQVWDNYNVLFGCG |
| | | TKLTVLGGSGGSQVQLVQSGAEVKKPG |
| | | ASVKVSCKASGYTFTSYYMHWVRQAPG |
| | | QCLEWMGAIEPTYGSTSYAQKFQGRVT |
| | | MTRDTSTSTVYMELSSLRSEDTAVYYC |
| | | ARGSAYYYDFADYWGQGTLVTVSS |
| | IG-scDb_10 | EVQLVESGGGLVQPGGSLRLSCAASGV |
| | chain 1: | SLPDYGVSWVRQAPGKGLEWIGVIWGS |
| | | ETTYYNSALKSKFIISRDNAKNSLYLQ |
| | | MNSLRAEDTAVYYCARHYYYGGSYAMD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | ELFPPKPKDTLMISRTPEVTCVVVAVS |
| | | HEDPEVKENWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVESCSVMH |
| | | EALHNHYTQKSLSLSPGGGGGSGGGGS |
| | | SYELTQPLSVSVALGQTARITCGGHNI |
| | | GSKNVHWYQQKPGQAPVLVIYQDNKRP |
| | | SGIPERFSGSNSGNTATLTISRAQAGD |
| | | EADYYCQVWDNYNVLFGCGTKLTVLGG |
| | | SGGSQVQLVQSGAEVKKPGASVKVSCK |
| | | ASGYTFTSYYMHWVRQAPGQCLEWMGA |
| | | IEPTYGSTSYAQKFQGRVTMTRDTSTS |
| | | TVYMELSSLRSEDTAVYYCARGSAYYY |
| | | DFADYWGQGTLVTVSSGGSGGSGGSGG |
| | | SGGSGGSSYELTQPLSVSVALGQTARI |
| | | TCGGHNIGSKNVHWYQQKPGQAPVLVI |
| | | YQDNKRPSGIPERFSGSNSGNTATLTI |
| | | SRAQAGDEADYYCQVWDNYNVLEGCGT |
| | | KLTVLGGSGGSQVQLVQSGAEVKKPGA |
| | | SVKVSCKASGYTFTSYYMHWVRQAPGQ |
| | | CLEWMGAIEPTYGSTSYAQKFQGRVTM |
| | | TRDTSTSTVYMELSSLRSEDTAVYYCA |
| | | RGSAYYYDFADYWGQGTLVTVSS |
| 192 | IG-scDb_10 | DIQMTQSPSSLSASVGDRVTITCRASQ |
| | chain 2: | DISKYLNWYQQKPGKVPKLLIYHTSRL |
| | | HSGVPDRFSGSGSGTDFTLTISSLQPE |
| | | DVATYYCQQGNTLPYTFGQGTKVEIKR |
| | | TVAAPSVFIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |
| 193 | scFv-IgAb_398 | EVQLVESGGGLVQPGGSLRLSCAASGV |
| | chain 1: | SLPDYGVSWVRQAPGKGLEWIGVIWGS |
| | | ETTYYNSALKSKFIISRDNAKNSLYLQ |
| | | MNSLRAEDTAVYYCARHYYYGGSYAMD |
| | | YWGQGTLVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPEFEGGPSV |
| | | FLFPPKPKDTLMISRTPEVTCVVVAVS |
| | | HEDPEVKFNWYVDGVEVHNAKTKPREE |
| | | QYNSTYRVVSVLTVLHQDWLNGKEYKC |
| | | KVSNKALPAPIEKTISKAKGQPREPQV |
| | | YTLPPSREEMTKNQVSLTCLVKGFYPS |
| | | DIAVEWESNGQPENNYKTTPPVLDSDG |
| | | SFFLYSKLTVDKSRWQQGNVESCSVMH |
| | | EALHNHYTQKSLSLSPGGGGGSGGGGS |
| | | GGGGSGGGGSGGGGSGGGGSSYELTQP |
| | | LSVSVALGQTARITCGGHNIGSKNVHW |
| | | YQQKPGQAPVLVIYQDNKRPSGIPERF |
| | | SGSNSGNTATLTISRAQAGDEADYYCQ |
| | | VWDNYNVLFGCGTKLTVLGGSGGSGGS |
| | | GGSGGSGGSGGSQVQLVQSGAEVKKPG |
| | | ASVKVSCKASGYTFTSYYMHWVRQAPG |
| | | QCLEWMGAIEPTYGSTSYAQKFQGRVT |
| | | MTRDTSTSTVYMELSSLRSEDTAVYYC |
| | | ARGSAYYYDFADYWGQGTLVTVSS |
| 194 | scFv-IgAb_398 | DIQMTQSPSSLSASVGDRVTITCRASQ |
| | chain 2: | DISKYLNWYQQKPGKVPKLLIYHTSRL |
| | | HSGVPDRESGSGSGTDFTLTISSLQPE |
| | | DVATYYCQQGNTLPYTFGQGTKVEIKR |
| | | TVAAPSVEIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSENRGEC |

Embodiments illustratively described herein may suitably be practiced in the absence of any element or elements, limitation or limitations, not specifically disclosed herein. Thus, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group.

Equivalents: Those skilled in the art will recognize or be able to ascertain using no more than routine experimentation, many equivalents to the specific embodiments of the invention described herein. Such equivalents are intended to be encompassed by the following claims.

Further embodiments will become apparent from the following claims.

1. An antibody construct comprising
(i.) at least four first binding domains (A), wherein said first binding domain (A) is capable of specifically binding to a first target (A′) that is an immune-regulatory antigen on the surface of an innate immune effector cell, wherein the immune effector cell is a natural killer cell or a macrophage;
(ii.) a second binding domain (B), which is capable of specifically binding to a second target (B′) that is an antigen on the surface of a target cell; and
(iii) a fourth domain (D) comprising a half-life extension domain that comprises two CH3 domains,
wherein a first first binding domain and a second first binding domain that are fused to each other (A1A2) are fused to the C terminus of a first CH3 domain of the fourth domain (D), whereas a third first binding domain and a fourth first binding domain that are fused to each other (A3A4) are fused to the C terminus of a second CH3 domain of the fourth domain (D).

2. The antibody construct of claim 1, wherein the first target (A′) is selected from the group consisting of CD16A, CD56, NKG2A, NKG2D, NKp30, NKp44, NKp46, NKp80, DNAM-1 (CD226), SLAMF7 (CD319), CD244 (2B4), OX40, CD47, SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5, KIR3DS1, and CD3.

3. The antibody construct of claim 1 or 2, wherein the antibody construct comprises a third binding domain (C), which is capable of specifically binding to a third target (C′) that is an antigen on the surface of a target cell that is other than the second target (B′).

4. The antibody construct of any one of the preceding claims, wherein the second binding domain (B) comprises a VH and a VL domain of an antibody.

5. The antibody construct of any one of the preceding claims, wherein the second target (B′) is selected from the group consisting of CD19, CD20, CD22, CD30, CD33, CD52, CD70, CD74, CD79b, CD123, CLL1, BCMA, FCRH5, EGFR, EGFRvlll, HER2, and GD2.

6. The antibody construct of any one of the preceding claims, wherein the first binding domain (A) comprises a VH and a VL domain of an antibody.

7. The antibody construct of any one of the preceding claims, wherein the first target (A′) is CD16A.

8. The antibody construct of any one of the preceding claims, wherein the first binding domain (A) binds to an epitope on CD16A which is C-terminal to the physiological Fcγ receptor binding domain, said epitope preferably comprises Y158 of SEQ ID NO: 13.

9. The antibody construct of any one of the preceding claims, wherein a second binding domain (B) is fused to the N terminus of a hinge of the fourth domain (D).

10. The antibody construct of claim 9, wherein another second binding domain (B) is fused to the N terminus of another hinge of the fourth domain (D).

11. The antibody construct of any one of the preceding claims, wherein the first binding domain (A) comprises a VH region comprising CDR-H1, CDR-H2 and CDR-H3 and a VL region comprising CDR-L1, CDR-L2 and CDR-L3 selected from:
(a) a CDR-H1 as depicted in SEQ ID NO: 77, a CDR-H2 as depicted in SEQ ID NO: 78, a CDR-H3 as depicted in SEQ ID NO: 79, a CDR-L1 as depicted in SEQ ID NO: 80, a CDR-L2 as depicted in SEQ ID NO: 81, and a CDR-L3 as depicted in SEQ ID NO: 82;
(b) a CDR-H1 as depicted in SEQ ID NO: 83, a CDR-H2 as depicted in SEQ ID NO: 84, a CDR-H3 as depicted in SEQ ID NO: 85, a CDR-L1 as depicted in SEQ ID NO: 86, a CDR-L2 as depicted in SEQ ID NO: 87, and a CDR-L3 as depicted in SEQ ID NO: 88; and
(c) a CDR-H1 as depicted in SEQ ID NO: 77, a CDR-H2 as depicted in SEQ ID NO: 89, a CDR-H3 as depicted in SEQ ID NO: 79, a CDR-L1 as depicted in SEQ ID NO: 80, a CDR-L2 as depicted in SEQ ID NO: 81, and a CDR-L3 as depicted in SEQ ID NO: 82.

12. The antibody construct of any one of the preceding claims, having an amino acid sequence selected from the group consisting of SEQ ID NOs: 148, 149, 150 and 151, 152 and 153, 154 and 155, 156 and 157, 158 and 159, 160 and 161, 162 and 163, and 180-183, 190, and 191 and 192.

13. A nucleic acid molecule comprising a sequence encoding an antibody construct of any one of claims 1 to 12 or a vector comprising said nucleic acid molecule.

14. A host cell comprising a nucleic acid molecule or the vector of claim 13.

15. A method of producing an antibody construct of any one of claims 1 to 12, said method comprising culturing a host cell of claim 14 under conditions allowing the expression of the antibody construct of any one of claims 1 to 12 and optionally recovering the produced antibody construct from the culture.

16. A pharmaceutical composition comprising an antibody construct of any one of claims 1 to 12, or produced by the method of claim 15.

17. An antibody construct of any one of claims 1 to 12 for use in therapy.

18. An antibody construct of any one of claims 1 to 12, or produced by the method of claim 15, for use in the prevention, treatment or amelioration of a disease selected from a proliferative disease, a tumorous disease, a viral disease or an immunological disorder.

19. A method of treatment or amelioration of a proliferative disease, a tumorous disease, a viral disease or an immunological disorder, comprising the step of administering to a subject in need thereof the antibody construct of any one of claims 1 to 12, or produced by the method of claim 15.

20. Use of an antibody construct of any one of claims 1 to 12, or produced by the method of claim 15, for the preparation of a composition for the prevention, treatment or amelioration of a disease selected from a proliferative disease, a tumorous disease, a viral disease or an immunological disorder.

21. A kit comprising an antibody construct of any one of claims 1 to 12, or produced by the method of claim 15, a nucleic acid molecule of claim 13, a vector of claim 13, and/or a host cell of claim 14.
